(19) United States
(12) Patent Application Publication
(10) Pub. No.: US 2022/0062233 A1
(43) Pub. Date: Mar. 3, 2022

(54) Compounds for the Reduction of the Deleterious Activity of Extended Nucleotide Repeat Containing Genes

(71) Applicants: The Board of Trustees of the Leland Stanford Junior University, Stanford, CA (US); NATIONAL YANG MING CHIAO TUNG UNIVERSITY, TAIPEI (TW)

(72) Inventors: Thomas W. SUN, Redwood City, CA (US); Gary PROBST, Redwood City, CA (US); Jason BAIK, Redwood City, CA (US)

(21) Appl. No.: 17/414,095

(22) PCT Filed: Dec. 12, 2019
(86) PCT No.: PCT/US19/66022

§ 371 Date: Jun. 15, 2021

Related U.S. Application Data
(60) Provisional application No. 62/781,469, filed on Dec. 18, 2018.

(51) Int. Cl. A61K 31/404; C07D 209/88; C07D 403/04; C07D 401/12; C07D 409/12; C07F 5/02; C07D 471/04; A61P 25/28; A61K 31/4155; A61K 31/4439; A61K 31/69; A61K 31/437; A61K 31/5377; A61K 31/496

(52) U.S. Cl. CPC A61K 31/404; C07B 2200/05; C07D 403/04; C07D 401/12; C07D 409/12; C07F 5/025; C07D 471/04; A61P 25/28; A61K 31/4155; A61K 31/4439; A61K 31/69; A61K 31/437; A61K 31/5377; A61K 31/496; C07D 209/88

(57) ABSTRACT

Aspects of the present disclosure include methods of reducing the deleterious impact of a target gene in a cell, such as the deleterious activity of a mutant extended nucleotide repeat (NR) containing target gene in a cell by contacting the cell with an effective amount of a tetrahydrocarbazole compound. The deleterious activity (e.g., toxicity and/or dis-functionality of products encoded thereby) of a mutant extended NR containing target gene may be reduced, e.g., by reducing (and in some instances differentially, including selectively, reducing) the production or activity of toxic expression products (e.g., RNA or protein) encoded by the target gene. Kits and compositions for practicing the subject methods are also provided.

### CROSS-REFERENCE TO RELATED APPLICATIONS

Pursuant to 35 U.S.C. § 119(e), this application claims priority to the filing date of the U.S. Provisional Patent Application Ser. No. 62/781,469 filed Dec. 18, 2018; the disclosure of which application is herein incorporated by reference.

### INTRODUCTION

Abnormal expansion of nucleotide repeats in coding or non-coding DNA regions have been associated with many disease conditions. These mutant regions of expanded repeats may result in mutant gene products that cause disease through a variety of different mechanisms, e.g., loss- or gain-of-function mechanisms, e.g., as a result of toxic RNA, altered RNA processing, misfolded and abnormal proteins, reduced gene expression and altered protein function.

Long repeats may form unusual DNA structures that can increase the likelihood of expansion or sometimes contraction. Models explaining the dynamic behavior of repeat regions also involve slipped strand mispairing during DNA replication or repair, misalignment and excision repair, and unequal crossing-over. Due to somatic and germline instability of the repeat regions, families with repeat mutations may see an increase in disease severity and an earlier age of onset from one generation to the next, a phenomenon known as anticipation.

Certain trinucleotide repeat diseases result from repeats occurring in non-coding sequences, and such repeats may result in loss of function of the affected gene. Trinucleotide repeat sequences implicated in diseases include CGG, GCC, GAA, CTG, and CAG units. The nature of the sequence itself and the location of repeats can affect the mechanism of disease pathogenesis. X-linked trinucleotide diseases are Fragile X syndrome (FRAXA), Fragile XE MR (FRAXE) and Fragile X tremor/ataxia syndrome (FXTAS). This group of diseases includes both loss of function mutations and the production of toxic RNA. Autosomal diseases include myotonic dystrophy, Friedreich's ataxia and two types of spinocerebellar ataxia (SCA8 and SCA12). Phenotypic manifestations of a disease are highly variable, and pathogenic mechanisms also vary from disease to disease.

Polyglutamine repeat diseases are a particular trinucleotide repeat disease category. These diseases result from exonic repeats that are located in protein-coding regions of genes and code for polyglutamine tracts in the proteins encoded by these genes. Subsets of neurons are especially vulnerable to polyglutamine repeat disease mechanisms. The following examples are known polyglutamine repeat diseases: Dentatorubral-pallidoluysian atrophy (DRPLA), Huntington's disease, spinobulbar muscular dystrophy, and spinocerebellar ataxia types 1, 2, 3, 6, 7, and 17. Huntington's Disease-like 2 can result from pathogenic polyglutamine repeat mechanisms. Polyglutamine repeat diseases commonly produce symptoms that have an onset relatively late in life and lead to progressive neuronal dysfunction and ultimately, to severe neurodegeneration. A hallmark of these diseases is the presence of aggregates of proteins containing polyglutamine tracts, mainly found in the nucleus of affected neurons. Misfolded repeat containing proteins may be toxic, and protein aggregates may have altered interactions with transcriptional regulators. However, the exact pathogenic mechanism is complex. Not only do repeat expansions affect genes encoding proteins with dissimilar functions, but polyglutamine repeat diseases can also manifest in different regions of the brain. Polyglutamine repeat proteins may play a role in inappropriately activating a cell's apoptotic pathway, leading to cell death.

Nucleotide repeats encoding polyalanine tracts have also been found to cause disease. For example, trinucleotide repeats encoding alanine tracts have been linked to congenital malformation syndromes. Affected genes encode transcription factors that play roles during development, and the repeats lead to misfolded proteins and protein aggregation and degradation. Unstable regions of various other sizes of nucleotide repeat units are also the basis for disease. Tetranucleotide repeats cause myotonic dystrophy type 2, and pentanucleotide repeats result in SCA 10 and SCA 31. Dodecamer repeats have been implicated in progressive myoclonic epilepsy.

Expansion of trinucleotide repeats in gene segments that do not encode proteins can cause disease by producing abnormal RNAs. Furthermore, repeat expansions need not necessarily involve trinucleotides. For example, expansion of the GGGGCC hexanucleotide repeat in non-coding regions of C90RF72 is the most common cause of two diseases, autosomal-dominant frontotemporal dementia (FTD) and amyotrophic lateral sclerosis (ALS). Individuals afflicted with this autosomal dominant mutation experience deficits in executive function and behavioral changes (FTD) or motor neuron dysfunction (ALS). Some patients may have a combination of FTD and ALS symptoms. C90RF72 hexanucleotide repeats are also rarely associated with parkinsonism, pseudodementia, psychiatric disorders, and other neurological diseases. While the number of hexanucleotide repeats in C90RF72 normally is fewer than 25, mutant repeat regions can contain up to 1500 or more hexanucleotide units. Studies propose that the hexanucleotide repeat regions are unstable and that abnormally long repeats may occur on a predisposing haplotypic background prone to expansion.

### SUMMARY

Aspects of the present disclosure include methods of reducing the deleterious impact of a target gene in a cell, such as the deleterious activity of a mutant extended nucleotide repeat (NR) containing target gene in a cell, by contacting the cell with an effective amount of a tetrahydrocarbazole compound. The deleterious activity (e.g., toxicity and/or dis-functionality of products encoded thereby) of a mutant extended NR containing target gene may be reduced, e.g., by reducing (and in some instances differentially, including selectively, reducing) the production or activity of toxic expression products (e.g., RNA or protein) encoded by the target gene. Kits and compositions for practicing the subject methods are also provided.

### BRIEF DESCRIPTION OF THE DRAWINGS

FIG. 1A is a graph showing in vitro mouse hepatocyte stability of exemplary compounds. FIG. 1B is a graph showing blood concentration-time profile in mice after IV administration of 0.5 mg/kg or PO administration of 5 mg/kg of exemplary compounds.

### DEFINITIONS

Before describing exemplary embodiments in greater detail, the following definitions are set forth to illustrate and define the meaning and scope of the terms used in the description. Any undefined terms have their art recognized meanings.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Where compounds described herein contain one or more chiral centers and/or double-bond isomers (i.e., geometric isomers), enantiomers or diastereomers, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). In some cases, alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. In some cases, alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein. The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

“Amino” refers to the group —NH₂. The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The terms “substituted heterocycle”, “substituted heterocyclic”, “substituted heterocyclic group” and “substituted heterocyclo” refer to heterocycle, heterocyclic, and heterocyclo groups substituted with one or more groups preferably selected from alkyl, substituted alkyl, alkenyl, oxo, aryl, substituted aryl, heterocyclo, substituted heterocyclo, carbocyclo (optionally substituted), halo, hydroxy, alkoxy (optionally substituted), aryloxy (optionally substituted), alkanoyl (optionally substituted), aroyl (optionally substituted), alkylester (optionally substituted), arylester (optionally substituted), cyano, nitro, amido, amino, substituted amino, lactam, urea, urethane, sulfonyl, and the like, where optionally one or more pair of substituents together with the atoms to which they are bonded form a 3 to 7 member ring.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O) O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R¹⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified: —R⁶⁰, halo, μO⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂ M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S (O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —PO(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰) R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

“Pharmaceutically effective amount” and “therapeutically effective amount” refer to an amount of a compound sufficient to elicit the desired therapeutic effect (e.g., treatment of a specified disorder or disease or one or more of its symptoms and/or prevention of the occurrence of the disease or disorder). In reference to polyglutamine diseases, a pharmaceutically or therapeutically effective amount includes an amount sufficient to, among other things, prevent or cause a reduction of proteinaceous deposits in the brain of a subject.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

Also of interest as active agents for use in embodiments of the methods are prodrugs. Such prodrugs are in general functional derivatives of the compounds that are readily convertible in vivo into the required compounds. Thus, in the methods of the present disclosure, the term “administering” encompasses administering the compound specifically disclosed or with a compound which may not be specifically disclosed, but which converts to the specified compound in vivo after administration to the subject in need thereof. Conventional procedures for the selection and preparation of suitable prodrug derivatives are described, e.g., in Wermuth, “Designing Prodrugs and Bioprecursors” in Wermuth, ed. The Practice of Medicinal Chemistry, 2d Ed., pp. 561-586 (Academic Press 2003). Prodrugs include esters that hydrolyze in vivo (e.g., in the human body) to produce a compound described herein suitable for the methods and compositions of the present disclosure. Suitable ester groups include, without limitation, those derived from pharmaceutically acceptable, aliphatic carboxylic acids, particularly alkanoic, alkenoic, cycloalkanoic and alkanedioic acids, in which each alkyl or alkenyl moiety has no more than 6 carbon atoms. Illustrative esters include formates, acetates, propionates, butyrates, acrylates, citrates, succinates, and ethylsuccinates.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

Other definitions of terms may appear throughout the specification.

### DETAILED DESCRIPTION

As summarized above, aspects of the present disclosure include methods of reducing the deleterious impact of a target gene in a cell, such as the deleterious activity of a mutant extended nucleotide repeat (NR) containing target gene in a cell, by contacting the cell with an effective amount of a tetrahydrocarbazole compound of formula (I). The deleterious activity (e.g., toxicity and/or dis-functionality of products encoded thereby) of a mutant extended NR containing target gene may be reduced, e.g., by reducing (and in some instances differentially, including selectively, reducing) the production or activity of toxic expression products (e.g., RNA or protein) encoded by the target gene. Kits and compositions for practicing the subject methods are also provided. Methods, kits and compositions of the invention find use in a variety of different applications, including the prevention or treatment of disease conditions associated with the presence of genes containing mutant extended nucleotide repeats, e.g., mutant extended trinucleotide repeats, such as Huntington's Disease (HD).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Tetrahydrocarbazole Compounds

Aspects of the present disclosure include tetrahydrocarbazole compounds which find use in reduction of the deleterious impact in a cell of a target gene that includes an extended nucleotide repeat (NR). A tetrahydrocarbazole compound is a compound having a tetrahydro-1H-carbazole core structure, or a heteroatom substituted version thereof, that can be further substituted at any convenient position of the core structure. The subject compound can be a substituted 2,3,4,9-tetrahydro-1H-carbazole compounds, having a heteroatom at the 1 position, such as an 1-amino, 1-oxo or 1-thio, which is itself substituted with a substituted arylalkyl or substituted heteroarylalkyl. The 2,3,4,9-tetrahydro-1H-carbazole core structure can be further substituted at any convenient carbons of the carbazole ring structure.

In some embodiments, the tetrahydrocarbazole compound has a structure of formula (I):

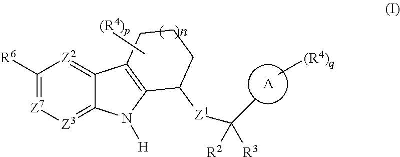

wherein:

A is aryl or heteroaryl;

Z¹ is NR¹, O or S, wherein R¹ is H, alkyl or substituted alkyl;

Z² is CR⁵ or N;

Z⁷ is CR⁷ or N;

Z³ is CR⁸ or N;

R² and R³ are independently selected from H, alkyl and substituted alkyl, or R² and R³ are cyclically linked and together with the carbon atom to which they are attached provide a 3-7 membered carbocycle or heterocycle ring that is optionally substituted;

each R⁴ and R⁵-R⁸ are independently selected from H, alkyl, substituted alkyl, alkoxy, substituted alkoxy, alkenyl, substituted alkenyl, alkynyl, substituted alkynyl, aryl, substituted aryl, heteroaryl, substituted heteroaryl, heterocycle, substituted heterocycle, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, boronic acid and boronate ester;

n is 0, 1 or 2; and

p and q are independently 0-5;

or a pharmaceutically acceptable salt thereof.

In some embodiments of formula (I), A is monocyclic or fused bicyclic aryl or monocyclic heteroaryl. In some instances of formula (I), A is selected from phenyl, naphthyl, imidazolyl, thiophene, furanyl, pyrrolyl, pyridyl, pyridazine, pyrimidine, pyrazine and substituted versions thereof. In certain instances of formula (I), A is selected from phenyl, 1-naphthyl, 2-naphthyl, 4-imidazolyl, 2-thiophene, 2-furanyl, 2-pyrrolyl, 2-pyridyl, 4-pyridyl and 3-pyridyl, 3-pyridazine, 4-pyrimidine, 2-pyrimidine, 5-pyrimidine, 2-pyrazine and substituted versions thereof.

In some cases, A is phenyl. In certain embodiments of formula (I), A is a substituted phenyl of formula (II):

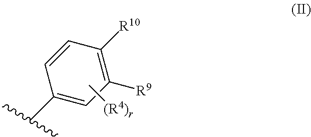

wherein:

r is 0-3; and

R⁹ and R¹⁰ are independently selected from H, alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring that is optionally further substituted.

In some cases, A is pyridyl. In certain embodiments of formula (I), A is a substituted pyridyl of one of formulae (IXa)-(IXd):

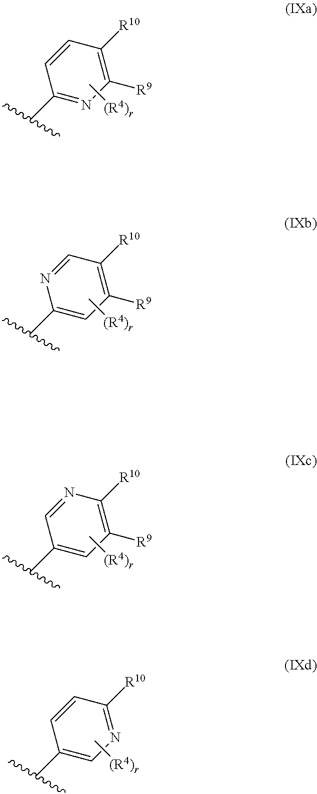

wherein:

r is 0-3 (e.g., 0, 1 or 2); and

R⁹ and R¹⁰ are independently selected from H, alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring that is optionally further substituted.

In some instances of formula (I), A is imidazolyl. In certain embodiments of formula (I), A is a substituted imidazolyl of formula (X):

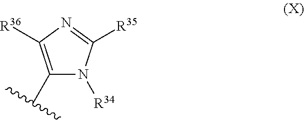

wherein:

R³⁵-R³⁶ are independently selected from H, alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle and substituted heterocycle; and

R³⁴ is selected from H, alkyl, substituted alkyl, alkylsulfonyl, substituted alkylsulfonyl, alkanoyl, substituted alkanoyl, alkyloxycarbonyl and substituted alkyloxycarbonyl.

In some instances of formula (I), A is thiophene. In certain embodiments of formula (I), A is a substituted thiophene of formula (XI):

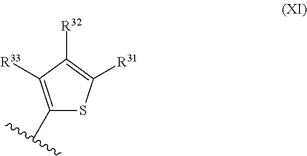

wherein R³¹-R³³ are independently selected from H, alkyl, substituted alkyl, alkoxy, substituted alkoxy, alkenyl, substituted alkenyl, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle and substituted heterocycle or R³¹ and R³² are cyclically linked and together with the carbon atoms to which they are attached provide a fused carbocyclic or heterocyclic ring that is optionally further substituted.

In some instances of formula (I)-(II) and (IXa)-(IXd), each R⁴ and R⁵-R¹⁰ are independently selected from H, C₁₋₆alkyl, substituted C₁₋₆alkyl (e.g., C₁₋₆alkoxy-C₁-alkyl, heterocyclyl-C₁₋₆alkyl, or substituted amino-C₁₋₆alkyl), C₁₋₆alkoxy, substituted C₁₋₆alkoxy, C₁₋₆alkenyl, substituted C₁₋₆alkenyl, C₁₋₆alkynyl, substituted C₁₋₆alkynyl, phenyl, substituted phenyl, heterocycle, substituted heterocycle, halogen, cyano, nitro, hydroxy, —NH₂, sulfoximine, N-substituted sulfoximine, carboxy, sulfonate, C₁₋₆alkanoyl, substituted C₁₋₆alkanoyl, C₁₋₆alkylsulfonamido, substituted C₁₋₆alkylsulfonamido, C₁₋₆alkylamido, substituted C₁₋₆alkylamido, C₁₋₆alkylamino, substituted C₁₋₆alkylamino, C₁₋₆alkyloxycarbonyl, substituted C₁₋₆alkyloxycarbonyl, boronic acid and boronate ester.

In certain instances of formula (II) and (IXa)-(IXd), R⁹ or R¹⁰ has one of the following structures:

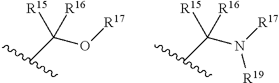

wherein:

R¹⁵ and R¹⁶ are independently selected from H, D, F, (C₁-C₆)alkyl and substituted (C₁-C₆)alkyl, or R¹⁵ and R¹⁶ are cyclically linked and together with the carbon atom to which they are connected provide cycloalkyl or substituted cycloalkyl ring;

R¹⁷ is H, alkyl or substituted alkyl; and

R¹⁸ and R¹⁹ are independently selected from H, alkyl, substituted alkyl, alkylsulfonyl, substituted alkylsulfonyl, alkanoyl, substituted alkanoyl, alkyloxycarbonyl and substituted alkyloxycarbonyl, or R¹⁸ and R¹⁹ are cyclically linked and together with the N atom to which they are attached provide a 5- or 6-membered heterocycle that is optionally further substituted.

In some embodiments, R¹⁷ is H. In some embodiments, R¹⁷ is (C₁-C₆)alkyl or substituted (C₁-C₆)alkyl. R¹⁷ can be a 2-substituted ethyl group. R¹⁷ can be a (C₂-C₆)alkyl substituted with hydroxy or (C₁-C₆)alkoxy. In certain embodiments of formula (II) and (IXa)-(IXd), R⁹ or R¹⁰ has one of the following structures:

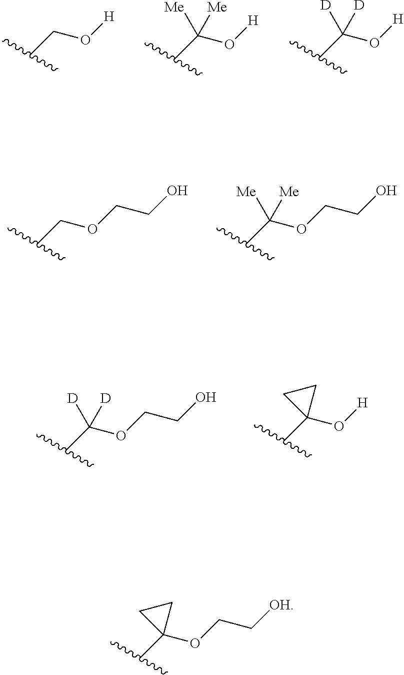

In some cases of formula (II) and (IXa)-(IXd), R¹⁸ and R¹⁹ are cyclically linked and together with the N atom to which they are attached provide a 5- or 6-membered heterocycle or substituted heterocycle. In some cases of formula (II) and (IXa)-(IXd), R⁹ or R¹⁰ has one of the following structures:

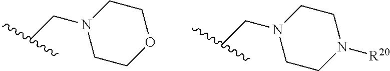

wherein R²⁰ is selected from H, alkyl, substituted alkyl, alkylsulfonyl, substituted alkylsulfonyl, alkanoyl, substituted alkanoyl, alkyloxycarbonyl and substituted alkyloxycarbonyl. In certain cases, R²⁰ is (C₁-C₆)alkanoyl. In some cases, R²⁰ is acetyl.

In certain embodiments of formula (II) and (IXa)-(IXd), R⁹ is a non-hydrogen substituent (e.g., as described herein) and R¹⁰ is H. In certain instances of formula (II) and (IXa)-(IXd), R⁹ is H and R¹⁰ is a non-hydrogen substituent (e.g., as described herein). In some embodiments of formula (II) and (IXa)-(IXd), R⁹ and R¹⁰ are each independently a non-hydrogen substituent (e.g., as described herein).

In certain instances of formula (II) and (IXa)-(IXd), R⁹ and R¹⁰ are cyclically linked and together with the carbon atoms to which they are attached provide a fused carbocyclic or heterocyclic ring that is optionally further substituted. The fused carbocyclic or heterocyclic ring can be a fused 6-membered aryl or heteroaryl. The fused carbocyclic or heterocyclic ring can be a fused 5-membered heterocycle. Exemplary 5-membered heterocycles of interest which may be fused to the aryl or heteroaryl ring of A include, but are not limited to, furan, thiophene, pyrrole, imidazole, pyrazole, oxazole, isooxazole, thiazole and isothiazole.

In some cases of formula (II), A is of formula (IIa):

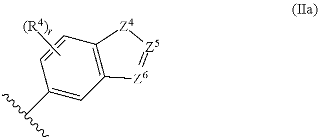

wherein:

Z⁴ is NR¹¹, O or S;

Z⁵ is CR¹² or N;

Z⁶ is CR¹³ or N;

R¹¹ is H, alkyl or substituted alkyl;

R¹² and R¹³ are independently selected from H, alkyl, substituted alkyl, alkoxy, substituted alkoxy, alkynyl, substituted alkynyl, halogen, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl and substituted alkyloxycarbonyl; and

r is 0-3.

In some cases of formula (IIa), Z⁴ is NR¹¹, Z⁵ is N and Z⁶ is CR¹³. In some cases of formula (IIa), Z⁴ is O, Z⁵ is N and Z⁶ is CR¹³. In some cases of formula (IIa), Z⁴ is S, Z⁵ is N and Z⁶ is CR¹³. In some cases of formula (IIa), Z⁴ is NR¹¹, Z⁵ is CR¹² and Z⁶ is CR¹³. In some cases of formula (IIa), Z⁴ is O, Z⁵ is CR¹² and Z⁶ is CR¹³. In some cases of formula (IIa), Z⁴ is S, Z⁵ is CR¹² and Z⁶ is CR¹³.

In some cases of formula (II), A is of formula (IIc):

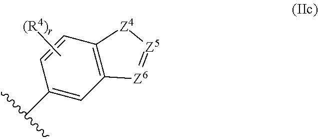

wherein:

Z⁴ is CR¹¹ or N;

Z⁵ is CR¹² or N;

Z⁶ is NR¹³, O or S;

R¹¹ is H, alkyl or substituted alkyl;

R¹² and R¹³ are independently selected from H, alkyl, substituted alkyl, alkoxy, substituted alkoxy, alkynyl, substituted alkynyl, halogen, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl and substituted alkyloxycarbonyl; and

r is 0-3.

In some cases of formula (IIc), Z⁶ is NR¹³, Z⁵ is N and Z⁴ is CR¹¹. In some cases of formula (IIc), Z⁶ is O, Z⁵ is N and Z⁴ is CR¹¹. In some cases of formula (IIa), Z⁶ is S, Z⁵ is N and Z⁴ is CR¹¹. In some cases of formula (IIc), Z⁶ is NR¹³, Z⁵ is CR¹² and Z⁴ is CR¹¹. In some cases of formula (IIc), Z⁶ is O, Z⁵ is CR¹² and Z⁴ is CR¹¹. In some cases of formula (IIc), Z⁶ is S, Z⁵ is CR¹² and Z⁴ is CR¹¹.

In certain instances of formula (II), R⁹ and R¹⁰ are cyclically linked and together with the carbon atoms to which they are attached provide a fused cyclic boronate ring that is optionally further substituted. The fused cyclic boronate ring can be a 5-membered or 6-membered ring. In some cases of formula (II), A is of formula (IIb):

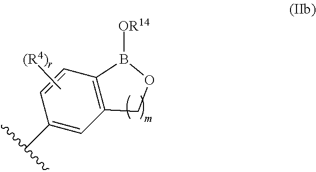

wherein: R¹⁴ is H, alkyl or substituted alkyl; m is 1 or 2; and r is 0-3.

In some embodiments of formula (I), R² and R³ are each H. In some embodiments of formula (I), R² or R³ is a (C₁-C₆)alkyl or substituted (C₁-C₆)alkyl. In some instances of formula (I), R² or R³ is —(CH₂)ₙ—R²¹, wherein R²¹ is halogen (e.g., fluoro) or (C₁-C₆)alkoxy (e.g., methoxy); and n is 1, 2 or 3. In some instances, R²¹ is fluoro. In certain cases, R²¹ is methoxy. In some cases of R² or R³, n is 1. In some cases of R² or R³, n is 2. In some cases of R² or R³, n is 3. In certain cases of formula (I), R² is H; and R³ is —CH₂—R²¹ wherein R²¹ is fluoro or methoxy.

In certain embodiments of formula (I), R² and R³ are cyclically linked and together with the carbon atom to which they are attached provide a cycloalkyl or substituted cycloalkyl. The substituted cycloalkyl can include one or more halogen substituents. The substituted cycloalkyl can include one or more substituents selected from alkyl, substituted alkyl, hydroxy, alkoxy and substituted alkoxy. In some instances, R² and R³ provide a cyclopropane or substituted cyclopropane ring. In some instances, R² and R³ provide a cyclobutene or substituted cyclobutene ring. In some instances, R² and R³ provide a cyclopentane or substituted cyclopentane ring. In some embodiments, the cycloalkyl or substituted cycloalkyl is of one of the following structures:

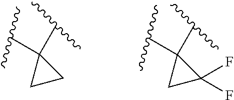 [SMILES: *C1(*)CC1 ; *C1(*)CC1(F)F]

In certain embodiments of formula (I), R² and R³ are cyclically linked and together with the carbon atom to which they are attached provide a heterocycle or substituted heterocycle. The heterocycle can be a 4-, 5- or 6-membered heterocycle. Heterocycles of interest include, but are not limited to, oxetane, thietane, azetidine, β-lactam, tetrahydrofuran, pyrrolidine, pyrrolidinone, 2-piperidinone, tetrahydropyran and piperidine. In certain embodiments of formula (I), R² and R³ are cyclically linked to provide a heterocycle of one of the following structures:

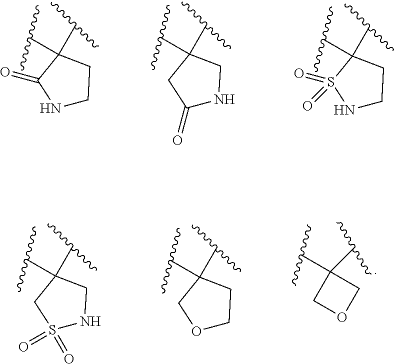 [SMILES: *C1(*)CCNC1=O ; *C1(*)CNC(=O)C1 ; *C1(*)CCNS1(=O)=O ; *C1(*)CNS(=O)(=O)C1 ; *C1(*)CCOC1 ; *C1(*)COC1]

In the various embodiments of formulae (I) described above, it is understood that the groups Z¹-Z³, Z⁷ and R¹-R⁸ if present, may be further defined according to any of the following embodiments.

In certain embodiments of formula (I), Z¹ is NH. In some embodiments of formula (I), Z¹ is NR¹ where R¹ is alkyl or substituted alkyl. In certain instances of formula (I), Z¹ is O. In certain cases of formula (I), Z¹ is S.

In certain embodiments of formula (I), Z² is CR⁵, Z⁷ is CR⁷ and Z³ is CR^{B}. In some cases of formula (I), Z² is N, Z⁷ is CR⁷ and Z³ is CR^{B}. In some instances of formula (I), Z² is CR⁵, Z⁷ is CR⁷ and Z³ is N. In some instances of formula (I), Z² is N, Z⁷ is CR⁷ and Z³ is N. In certain embodiments of formula (I), Z² is CR⁵, Z⁷ is N and Z³ is CR^{B}. In some cases of formula (I), Z² is N, Z⁷ is N and Z³ is CR^{B}. In some instances of formula (I), Z² is CR⁵, Z⁷ is N and Z³ is N. In some cases, R⁵ is H. In some cases, R⁷ is H. In some cases, R¹ is H. In certain instances, R⁷, R⁵ and R⁸ are each H.

In certain instances of formula (I), R⁶ is halogen, alkynyl or substituted alkynyl. In some instances of formula (I), R⁶ is halogen, e.g., chloro, iodo, bromo or fluoro. R⁶ can be Cl, I or Br. In certain cases of formula (I), R⁶ is Br. In some instances of formula (I), R⁶ is —CCH. In some cases of formula (I), R⁶ is —CC—CH₂OH.

In certain instances of formula (I), n is 0. In certain instances of formula (I), n is 1. In certain instances of formula (I), n is 2.

In certain instances of any of the embodiments of formula (I) described herein, the compound is enantiomerically enriched in a (1R) stereoisomer. In certain instances of formula (I), the compound is a (1R) stereoisomer.

In some embodiments of formula (I), the compound is of formulae (III):

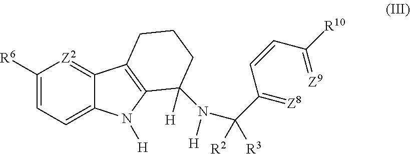

wherein:

Z² is N or CH;

Z⁸ is N or CH;

Z⁹ is N or CR⁹;

R² and R³ are independently selected from H, alkyl and substituted alkyl, or R² and R³ are cyclically linked and together with the carbon atom to which they are attached provide a 3-7 membered carbocycle or heterocycle ring;

R⁶ is selected from halogen, alkynyl and substituted alkynyl;

R⁹ and R¹⁰ are independently selected from H, alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring,

wherein at least one of R⁹ and R¹⁰ is not hydrogen; or wherein at least one of Z⁸ and Z⁹ is not N.

In certain instances of formula (III) and (XIII), the compound is enantiomerically enriched in a (1R) stereoisomer or is a (1R) stereoisomer of the formula:

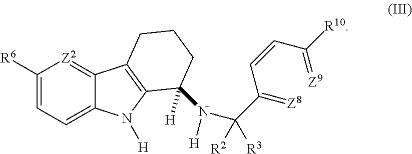

In some instances of formula (III) and (XIII), the compound is one of formulae (IIIa)-(IIIc):

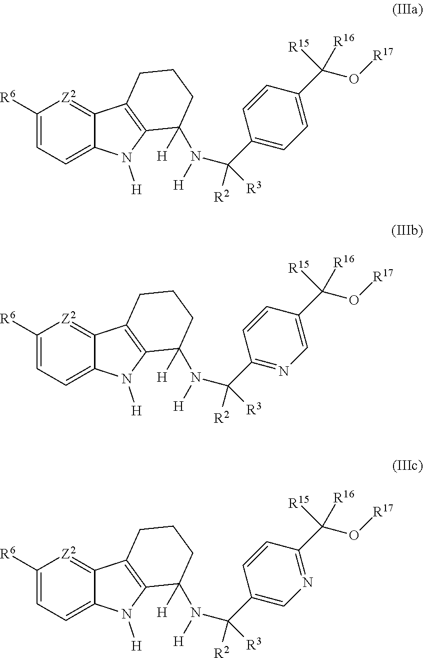

wherein:

Z² is N or CH;

R¹⁷ is H, alkyl or substituted alkyl; and

R¹⁵ and R¹⁶ are independently selected from H, D, F, (C₁-C₆)alkyl and substituted (C₁-C₆)alkyl, or R¹⁵ and R¹⁶ are cyclically linked to provide a cycloalkyl or substituted cycloalkyl.

In some instances of formulae (IIIa)-(IIIc), the compound is enantiomerically enriched in a (1R) stereoisomer or is a (1R) stereoisomer of one of the formula:

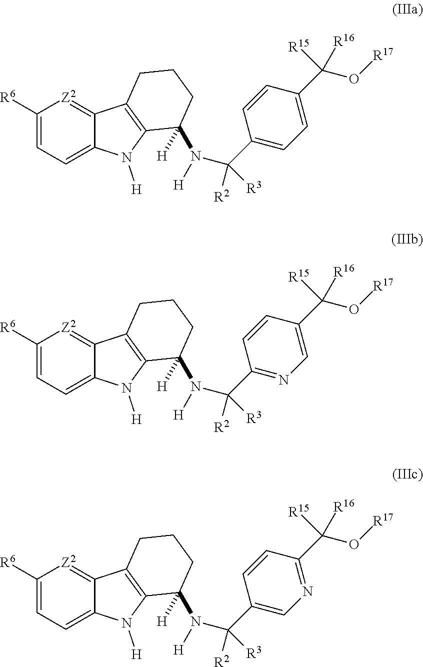

wherein:

Z² is N or CH;

R¹⁷ is H, alkyl or substituted alkyl; and

R¹⁵ and R¹⁶ are independently selected from H, D, F, (C₁-C₆)alkyl and substituted (C₁-C₆)alkyl, or R¹⁵ and R¹⁶ are cyclically linked to provide a cycloalkyl or substituted cycloalkyl.

In some embodiments of formula (IIIa)-(IIIc), R¹⁵ and R¹⁶ are each H. In certain embodiments of formula (IIIa)-(IIIc), R¹⁵ and R¹⁶ are each D. In some instances of formula (IIIa)-(IIIc), R¹⁵ and R¹⁶ are each (C₁-C₆)alkyl (e.g., methyl). In some cases of formula (IIIa)-(IIIc), R¹⁷ is H. In certain instances of formula (IIIa)-(IIIc), R¹⁷ is (C₁-C₆)alkyl. In some cases of formula (IIIa)-(IIIc), R¹⁷ is substituted (C₁-C₆)alkyl. In certain cases, R¹⁷ is CH₂CH₂OH. In some embodiments of formula (IIIa)-(IIIc), R¹⁵ and R¹⁶ are cyclically linked to provide a cyclopropyl or substituted cyclopropyl.

In some embodiments of formula (IIIa)-(IIIc), Z² is N. In some embodiments of formula (IIIa)-(IIIc), Z² is CH.

In some embodiments of formula (III), (XIII) and (IIIa)-(IIIc), R² and R³ are each H. In certain embodiments of formula (III), (XIII) and (IIIa)-(IIIc), R² is (C₁-C₆)alkyl or substituted (C₁-C₆)alkyl and R³ is H. In some embodiments of formula (III), (XIII) and (IIIa)-(IIIc), R² is (C₁-C₆)alkoxy-(C₁-C₆)alkyl and R³ is H. In some embodiments of formula (III), (XIII) and (IIIa)-(IIIc), R² is methoxymethyl and R³ is H.

In some embodiments of formula (III), (XIII) and (IIIa)-(IIIc), R⁶ is halogen. In some embodiments of formula (III), (XIII) and (IIIa)-(IIIc), R⁶ is Br. In some embodiments of formula (III), (XIII) and (IIIa)-(IIIc), R⁶ is alkynyl. In some embodiments of formula (III), (XIII) and (IIIa)-(IIIc), R⁶ is —CCH.

In some embodiments of formula (I)-(IIIc), the compound has one of the following structures:

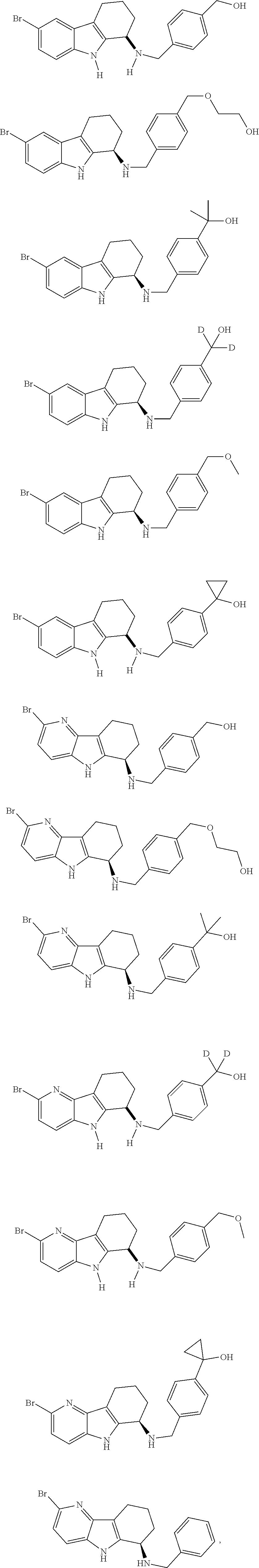

or a pharmaceutically acceptable salt thereof.

In some embodiments of Formulae (I)-(IIIc), wherein the compound is of one of formulae (IVa)-(IVc):

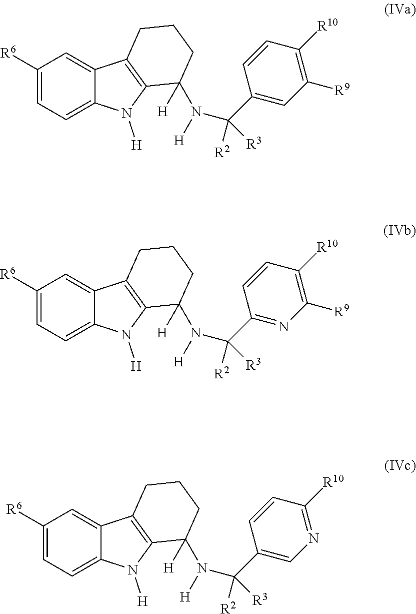

wherein:

R⁹ and R¹⁰ are independently selected from H, —NR^{a}R^{b}, alkoxy, substituted alkoxy, cyano, nitro, halogen, hydroxy, —CONR^{a}R^{b}, —SO₂NR^{a}R^{b}, —CO₂H, —SO₃H, alkylsulfonyl, substituted alkylsulfonyl, alkanoyl, substituted alkanoyl, alkylsulfonamido, substituted alkylsulfonamido, alkylamido, substituted alkylamido, alkyloxycarbonyl, substituted alkyloxycarbonyl, heterocycle, substituted heterocycle, boronic acid and boronate ester; and Ra and R^{b} are independently selected from H, alkyl and substituted alkyl, or Ra and R^{b} are cyclically linked and together with the N atom to which they are attached provide a 5- or 6-membered heterocycle that is optionally further substituted.

In some embodiments of Formulae (IVa)-(IVc), the compound is enantiomerically enriched in a (1R) stereoisomer or is a (1R) stereoisomer of one of the formulae:

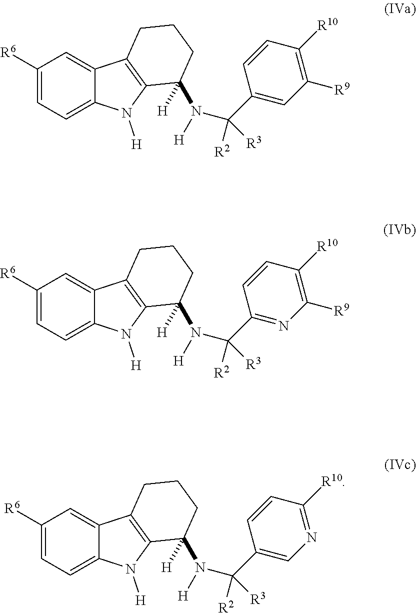

In some embodiments of Formulae (IVa)-(IVc), either R⁹ is H and R¹⁰ is —NR^{a}R^{b}; or R⁹ is —NR^{a}R^{b} and R¹⁰ is H. In certain embodiments of Formulae (IVa)-(IVc), R⁹ is H and R¹⁰ is alkoxy or substituted alkoxy. In some instances of Formulae (IVa)-(IVc), R⁹ is alkoxy or substituted alkoxy and R¹⁰ is H. In some cases of Formulae (IVa)-(IVc), R⁹ and R¹⁰ are selected from H, cyano, nitro, halogen, —CO₂H and —SO₃H. In certain instances of Formulae (IVa)-(IVc), R⁹ and R¹⁰ are selected from H and —B(OR)₂ wherein each R is independently H, alkyl or substituted alkyl.

In some embodiments of formulae (IVa)-(IVc), R² is (C₁-C₆)alkyl or substituted (C₁-C₆)alkyl and R³ is H. In some embodiments of formulae (IVa)-(IVc), R² is (C₁-C₆)alkoxy-(C₁-C₆)alkyl and R³ is H. In some embodiments of formulae (IVa)-(IVc), R² is methoxymethyl and R³ is H. In certain embodiments of Formulae (IVa)-(IVc), R² and R³ are each H. In some embodiments of formulae (IVa)-(IVc), R⁶ is halogen. In some embodiments of formulae (IVa)-(IVc), R⁶ is Br. In some embodiments of formulae (IVa)-(IVc), R⁶ is alkynyl. In some embodiments of formulae (IVa)-(IVc), R⁶ is —CCH.

In some embodiments of formulae (IVa)-(IVc), R¹⁰ is —NH₂ or —NMe₂ and R², R³ and R⁹ are each H. In some embodiments of formulae (IVa)-(IVc), R¹⁰ is N-pyrrolidine or substituted N-pyrrolidine and R², R³ and R⁹ are each H. In some embodiments of formulae (IVa)-(IVc), R⁹ is methoxy and R², R³ and R¹⁰ are each H. In some embodiments of formulae (IVa)-(IVc), R¹⁰ is methoxy and R², R³ and R⁹ are each H. In some embodiments of formulae (IVa)-(IVc), R¹⁰ is cyano and R², R³ and R⁹ are each H. In some embodiments of formulae (IVa)-(IVc), R¹⁰ is nitro and R², R³ and R⁹ are each H. In some embodiments of formulae (IVa)-(IVc), R¹⁰ is B(OR)₂ and R², R³ and R⁹ are each H, wherein each R is H, (C₁-C₆)alkyl or substituted (C₁-C₆)alkyl.

In some embodiments of formula (I) and (IVa)-(IVc), the compound has one of the following structures:

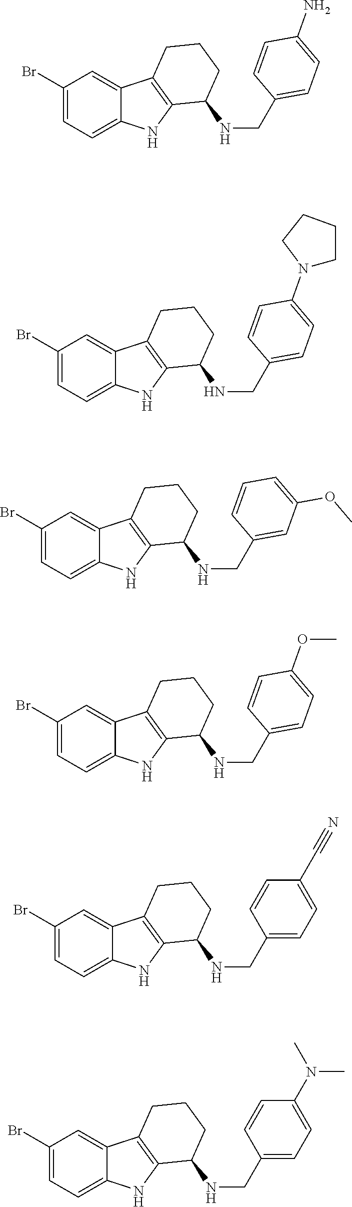 [SMILES: Nc1ccc(CN[C@@H]2CCCc3c2[nH]c2ccc(Br)cc32)cc1 ; Brc1ccc2[nH]c3c(c2c1)CCC[C@H]3NCc1ccc(N2CCCC2)cc1 ; COc1cccc(CN[C@@H]2CCCc3c2[nH]c2ccc(Br)cc32)c1 ; COc1ccc(CN[C@@H]2CCCc3c2[nH]c2ccc(Br)cc32)cc1 ; N#Cc1ccc(CN[C@@H]2CCCc3c2[nH]c2ccc(Br)cc32)cc1 ; CN(C)c1ccc(CN[C@@H]2CCCc3c2[nH]c2ccc(Br)cc32)cc1]

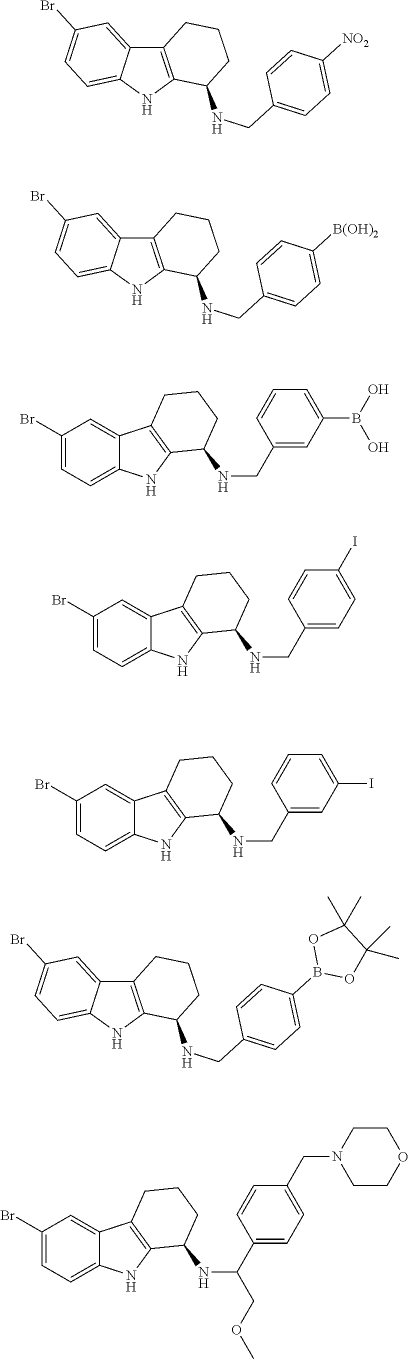

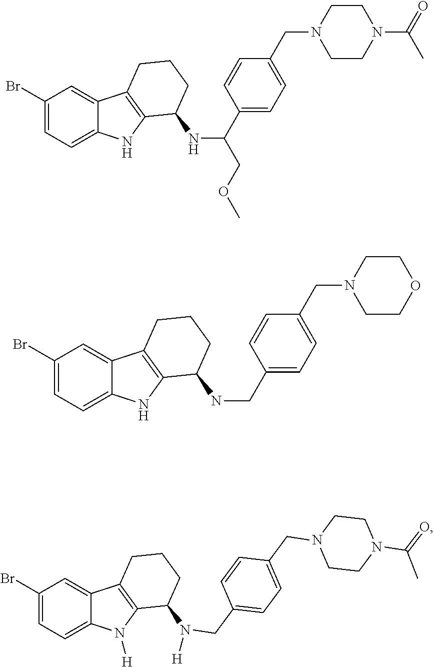 [SMILES: COCC(N[C@@H]1CCCc2c1[nH]c1ccc(Br)cc21)c1ccc(CN2CCN(C(C)=O)CC2)cc1 ; Brc1ccc2[nH]c3c(c2c1)CCC[C@H]3NCc1ccc(CN2CCOCC2)cc1 ; CC(=O)N1CCN(Cc2ccc(CN[C@@H]3CCCc4c3[nH]c3ccc(Br)cc43)cc2)CC1]

or a pharmaceutically acceptable salt thereof.

In some embodiments of formula (I), the compound is of one of formulae (Va)-(VIII):

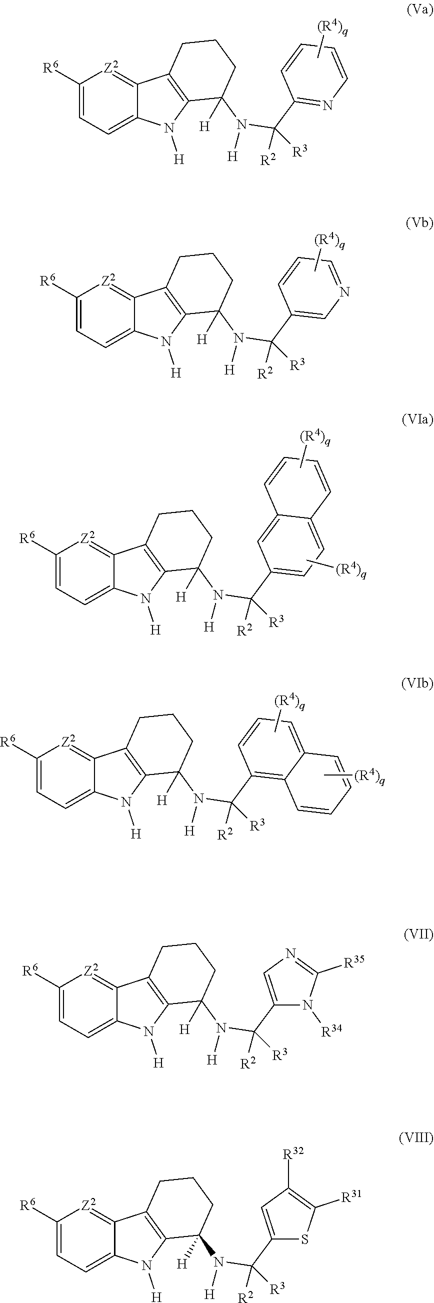

wherein:

Z² is N or CH;

each R⁴, R³¹, R³² and R³⁵ are independently selected from H, alkyl, substituted alkyl, alkoxy, substituted alkoxy, alkenyl, substituted alkenyl, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle and substituted heterocycle, or R³¹ and R³² are cyclically linked and together with the carbon atoms to which they are attached provide a fused carbocyclic or heterocyclic ring that is optionally further substituted; and

R³⁴ is selected from H, alkyl, substituted alkyl, alkylsulfonyl, substituted alkylsulfonyl, alkanoyl, substituted alkanoyl, alkyloxycarbonyl and substituted alkyloxycarbonyl.

In some embodiments of formulae (Va)-(VIII), the compound is enantiomerically enriched in a (1R) stereoisomer or is a (1R) stereoisomer of one of the formulae

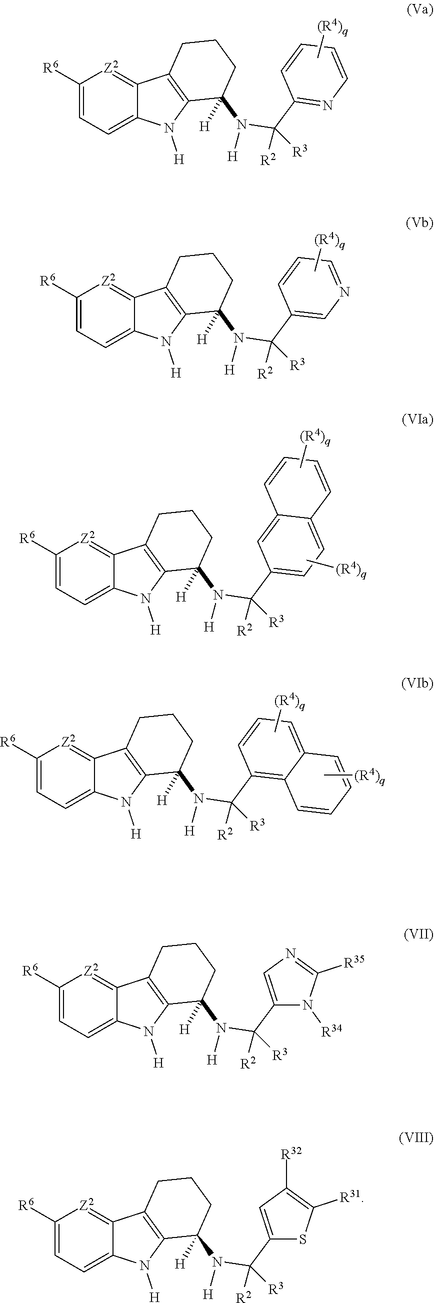

In some embodiments of formulae (Va)-(VIII), R² is (C₁-C₆)alkyl or substituted (C₁-C₆)alkyl and R³ is H. In some embodiments of formulae (IVa)-(IVc), R² is (C₁-C₆)alkoxy-(C₁-C₆)alkyl and R³ is H. In some embodiments of formulae (IVa)-(IVc), R² is methoxymethyl and R³ is H. In certain embodiments of formulae (Va)-(VIII), R² and R³ are each H. In some embodiments of formulae (Va)-(VIII), R⁶ is halogen. In some embodiments of formulae (Va)-(VIII), R⁶ is Br. In some embodiments of formulae (Va)-(VIII), R⁶ is alkynyl. In some embodiments of formulae (Va)-(VIII), R⁶ is —CCH. In some embodiments of formulae (Va)-(VIII), Z² is N. In some embodiments of formulae (Va)-(VIII), Z² is CH.

In some embodiments of formulae (Va)-(Vb), q is 0. In some embodiments of formulae (VIa)-(VIb), q is 0. In some embodiments of formula (VII), R²⁴ is alkyl or substituted alkyl and R²³H. In some embodiments of formula (VII), R²⁴ is methyl. In some embodiments of formula (VIII), R²² is alkyl or substituted alkyl and R²¹H. In certain embodiments of formulae (Va)-(VIII), R² and R³ are each H.

In some instances of formula (VIII), R³² is H. In some instances of formula (VIII), R³¹ is selected from (C₁-C₆)alkyl, substituted (C₁-C₆)alkyl, (C₁-C₆)alkenyl and substituted (C₁-C₆)alkenyl. In some instances of formula (VIII), R³¹ is —C(R⁴¹)₂OR⁴², where R⁴¹ and R⁴² are independently H or (C₁-C₆)alkyl, or the two R⁴¹ group are cyclically linked together to provide a cycloalkyl or substituted cycloalkyl. In some instances of formula (VIII), R³¹ is selected from —CH₂OH, —CH(CH₃)OH, —C(CH₃)₂OH, -isopropyl, —C(═CH₂)CH₃ and

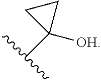 [SMILES: *C1(O)CC1]

In some embodiments of formulae (Va)-(VIII), the compound is of one of the following structures:

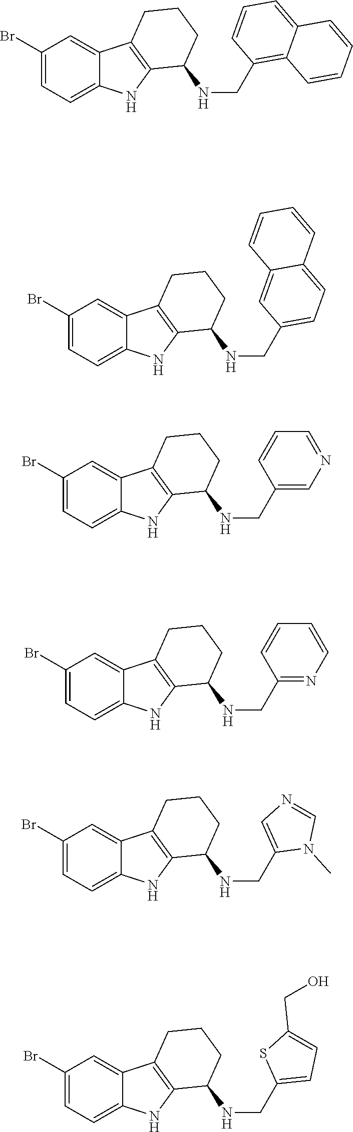 [SMILES: Brc1ccc2[nH]c3c(c2c1)CCC[C@H]3NCc1cccc2ccccc12 ; Brc1ccc2[nH]c3c(c2c1)CCC[C@H]3NCc1ccc2ccccc2c1 ; Brc1ccc2[nH]c3c(c2c1)CCC[C@H]3NCc1cccnc1 ; Brc1ccc2[nH]c3c(c2c1)CCC[C@H]3NCc1ccccn1 ; Cn1cncc1CN[C@@H]1CCCc2c1[nH]c1ccc(Br)cc21 ; OCc1ccc(CN[C@@H]2CCCc3c2[nH]c2ccc(Br)cc32)s1]

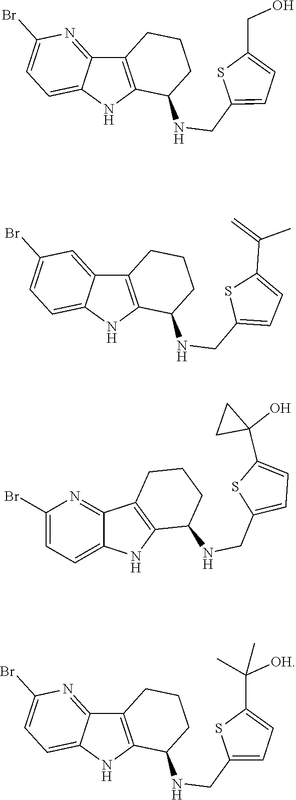 [SMILES: OCc1ccc(CN[C@@H]2CCCc3c2[nH]c2ccc(Br)nc32)s1 ; C=C(C)c1ccc(CN[C@@H]2CCCc3c2[nH]c2ccc(Br)cc32)s1 ; OC1(c2ccc(CN[C@@H]3CCCc4c3[nH]c3ccc(Br)nc43)s2)CC1 ; CC(C)(O)c1ccc(CN[C@@H]2CCCc3c2[nH]c2ccc(Br)nc32)s1]

In some embodiments, the tetrahydrocarbazole compound is NOT one of the following compounds, or a (1R) stereoisomer thereof:

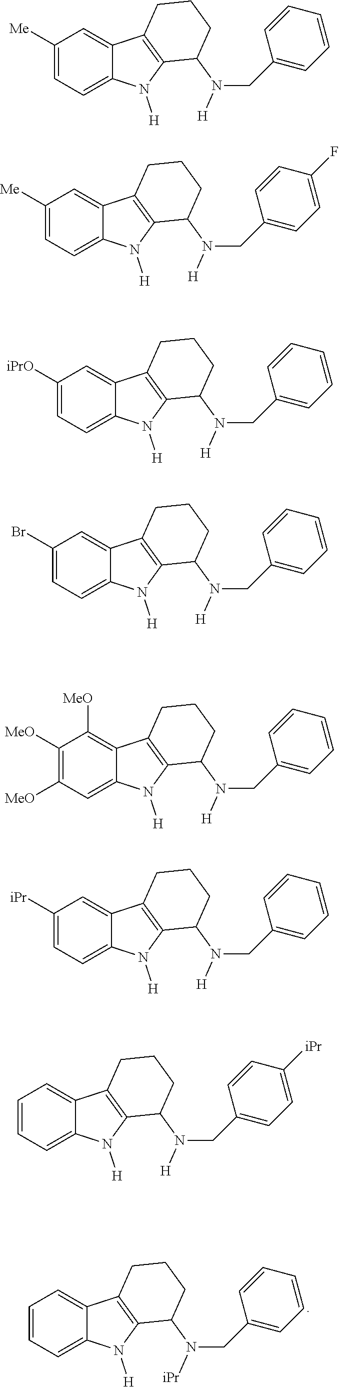

In certain instances of the formulae described herein, a tetrahydrocarbazole compound of interest, e.g., a compound that finds use in the applications described herein, is one of the compounds 1-74 of Table 2. It is understood that for any one of the compounds described herein that has a carbon atom at the Z² position of the scaffold (i.e., Z²═CH in formula (I)), that a corresponding compound is also described where the carbon atom is substituted with a nitrogen atom (i.e., Z²═N in formula (I)). See e.g., compounds 63-67 and 69-71. Derivatives of compounds 1-62, 68, 72 and 73 including a Z²═N substitution are also disclosed herein.

Aspects of the present disclosure include tetrahydrocarbazole compounds (e.g., as described herein), salts thereof (e.g., pharmaceutically acceptable salts), and/or solvate, hydrate and/or prodrug forms thereof. In addition, it is understood that, in any compound described herein having one or more chiral centers (e.g., a 1-amino tetrahydrocarbazole carbon center), if an absolute stereochemistry is not expressly indicated, then each center may independently be of R-configuration or S-configuration or a mixture thereof, e.g., a racemic mixture. In some embodiments of the tetrahydrocarbazole compounds described herein, the compound is enantiomerically enriched in a (1R) stereoisomer or is a (1R) stereoisomer.

It will be appreciated that all permutations of salts, solvates, hydrates, prodrugs and stereoisomers are meant to be encompassed by the present disclosure. In some embodiments, the subject compounds, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

### Pharmaceutical Preparations

Also provided are pharmaceutical preparations. Pharmaceutical preparations are compositions that include a tetrahydrocarbazole compound (e.g., as described herein) (for example one or more of the subject compounds, either alone or in the presence of one or more additional active agents) present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the present disclosure is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used.

When administered to a mammal, the compounds and compositions of the present disclosure and pharmaceutically acceptable vehicles, excipients, or diluents may be sterile. In some instances, an aqueous medium is employed as a vehicle when the subject compound is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of excipient will be determined in part by the particular compound, as well as by the particular method used to administer the composition. Accordingly, there is a wide variety of suitable formulations of the subject pharmaceutical compositions.

Administration of the subject compounds may be systemic or local. In certain embodiments administration to a mammal will result in systemic release of a compound of the present disclosure (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the compounds and compositions of the present disclosure are administered subcutaneously. In certain embodiments, the compounds and compositions of the present disclosure are administered orally. In certain embodiments, it may be desirable to administer one or more compounds of the present disclosure locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

A subject compound may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms of the present disclosure depend on the particular compound employed and the effect to be achieved, and the pharmacodynamics associated with each compound in the host. In pharmaceutical dosage forms, the compounds may be administered in the form of a free base, their pharmaceutically acceptable salts, or they may also be used alone or in appropriate association, as well as in combination, with other pharmaceutically active compounds.

Dose levels can vary as a function of the specific compound, the nature of the delivery vehicle, and the like. Desired dosages for a given compound are readily determinable by a variety of means. The dose administered to an animal, particularly a human, in the context of the present disclosure should be sufficient to effect a prophylactic or therapeutic response in the animal over a reasonable time frame, e.g., as described in greater detail herein. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

### Methods

Aspects of the present disclosure include methods for reducing the deleterious impact in a cell of a target gene that includes an extended nucleotide repeat (NR) by contacting the cell with an effective amount of a subject tetrahydrocarbazole compound (e.g., as described herein). Further aspects of the methods in which the subject compounds find use are described by Cohen et al. in WO 2016/196012, the disclosure of which is herein incorporated by reference in its entirety. Embodiments of the present disclosure include methods of reducing an extended nucleotide repeat-containing target gene's deleterious (e.g., harmful or injurious) activity in a cell. As used herein, the term “deleterious impact” refers to a harmful or injurious activity associated with, or attributable to, a target gene and any undesirable effect on the cell which may result from such activity. As used herein, the term “deleterious activity” refers to a harmful or injurious activity associated with, or attributable to, a target gene. By “reducing deleterious impact” or “reducing deleterious activity” is meant that the level of a harmful or injurious activity, or an undesirable effect thereof, is reduced by a statistically significant amount, and in some instances by 2-fold or more, such as by 5-fold or more, by 10-fold or more, by 20-fold or more, by 50-fold or more, by 100-fold or more, or even more, as compared to a control, e.g., a cell not contacted with the subject compound of interest. In some case, by “reducing deleterious impact” or “reducing deleterious activity” is meant that the level of a harmful or injurious activity, or an undesirable effect thereof, is reduced by a statistically significant amount, and in some instances by 10% or more, such as by 20% or more, by 30% or more, by 40% or more, by 50% or more, by 60% or more, by 70% or more, by 80% or more, by 90% or more, by 95% or more, by 99% or more, as compared to a control, e.g., a cell not contacted with the subject compound of interest. The deleterious impact or activity of the target gene that is reduced by the subject compounds may vary, and may include, but is not limited to, cell toxicity, reduction in cell viability, loss of cellular function, formation of protein aggregates, etc. The subject methods and compounds may reduce the deleterious impact or activity of the target gene in a cell, via a method as described by Cheng, Cohen et al. “Selective reduction of the deleterious activity of extended tri-nucleotide repeat containing genes” WO 2012078906, and Cohen et al. WO 2016196012, the disclosures of which are herein incorporated by reference in their entirety.

In certain embodiments, the methods may reduce the deleterious impact of an extended NR containing target gene by differentially reducing the deleterious impact of the target gene. In some embodiments, the subject compound modulates expression of the RNA and/or protein from the gene, such that it changes the expression of the RNA or protein from the target gene in some manner. In certain embodiments of the method, the subject compound modulates expression of the protein from the target gene. In certain cases of the method, the subject compound differentially, and in some instances selectively, reduces transcription of the target gene to reduce toxicity in the cell of a protein encoded by the target gene. Any convenient assays may be used to determine a reduction in transcription in a cell using the subject compounds relative to a control, e.g., a cell not contacted with the compound of interest, where the magnitude of transcription reduction may be 10% or more, such as 20% or more, 30% or more, 50% or more, 100% or more, such as by 2-fold or more, by 5-fold or more, by 10-fold or more, by 20-fold or more, by 50-fold or more, by 100-fold or more, or even more. In some instances of the method, the subject compound differentially, and in some instances selectively, reduces transcription of the target gene to enhance functionality of the protein in the cell. By enhance functionality is meant that a natural, desirable function or activity of a protein encoded by the target gene is increased relative to a control, e.g., a cell not contacted with the compound of interest, by 10% or more, such as 20% or more, 30% or more, 40% or more, 50% or more, 60% or more, 70% or more, 80% or more, 90% or more, 100% or more, such as by 2-fold or more, by 5-fold or more, by 10-fold or more, by 20-fold or more, by 50-fold or more, by 100-fold or more, or even more. Any convenient assays may be utilized to determine the level of function or activity of a protein of interest. By differentially reducing transcription of the target gene is meant that transcription of the target gene is reduced to an extent that is greater than any reduction of the non-target, e.g., corresponding wild-type, gene. The magnitude of any difference in transcription resulting from administration of the compound may vary, where in some instances the magnitude of reduction of target gene transcription relative to corresponding non-target gene transcription is 2-fold or more, by 5-fold or more, by 10-fold or more, by 20-fold or more, by 50-fold or more, by 100-fold or more, or even more. In some instances, while transcription of the target gene is reduced, administration of the compound results in substantially little, if any, transcription reduction of the corresponding non-target gene. In such instances, administration of the compound may be viewed as selectively reducing transcription of the target gene.

In certain embodiments, the methods may reduce the deleterious impact of an extended NR containing target gene by selectively reducing the deleterious impact of the target gene. As the methods of these embodiments are methods of selectively reducing the deleterious impact, i.e., activity, of the target gene, they do so while retaining at least a statistically measurable amount of normal or wild-type, e.g., beneficial, activity of the target gene, by which is meant the activity of the gene as present in normal or wild-type cells, which are cells in which the target gene does not include mutant extended nucleotide repeats (e.g., trinucleotide repeats) that give rise to deleterious activity. Accordingly, in these embodiments the subject methods may maintain or restore a physiologically desirable activity of the target gene despite the selective reduction of the harmful activity of the target gene. In some instances of the method, the compound modulates the activity of a protein encoded by the target gene. In some embodiments of the method, the expression of the protein from the target gene is selectively modulated relative to expression from a normal allele of the target gene (e.g., a normal allele of the target gene includes 8 to 25 CAG repeats). In certain cases, the activity of a normal allele of the target gene is maintained in the cell, e.g., has an activity that is within 20% (such as within 10%, within 5%, within 2% or within 1%) of the corresponding activity of a control cell not contacted with the compound of interest.

In yet other embodiments, the methods may reduce the deleterious impact in a cell of an extended NR containing target gene by reducing the deleterious impact as well as any normal activity of the target gene. As the methods of these embodiments are methods of non-selectively reducing the deleterious impact, i.e., activity, of the target gene, they reduce the deleterious impact of the target gene while also reducing to some extent, if not completely, the normal or wild-type, e.g., beneficial, activity of the target gene, by which is meant the activity of the gene as present in normal or wild-type cells, which are cells in which the target gene does not include mutant extended nucleotide repeats (e.g., TNRs) that give rise to deleterious activity.

In some cases, the harmful or injurious activity is a dysfunction of a protein product encoded by the target gene, where the dysfunction refers to an undesirable activity (e.g., cell toxicity) of the protein product that is not present in a normal allele of the target gene. In some instances, a target gene that does not include mutant extended nucleotide repeats that give rise to deleterious activity is referred to as a normal allele of the target gene. The normal allele of the target gene may include a desirable number of nucleotide repeats (NRs). In certain instances where the NR is a TNR, the normal allele includes 25 or less tri-nucleotide repeats (TNRs), such as 20 or less or 10 or less TNRs. In certain cases, the normal allele of the target gene includes 8 to 25 TNRs. In some instances, the normal allele includes 8 to 25 CAG repeats.

In certain embodiments of the method, the deleterious impact of the target gene is toxicity of the protein and the compound reduces the toxicity of the protein in the cell. In some instances, toxicity is a result of undesirable protein aggregation. As such, in some instances the subject methods result in a reduction in toxicity that is attributable to the target gene, where the magnitude of the toxicity reduction may vary, and in some instances is 2-fold or greater, such as by 5-fold or greater, by 10-fold or greater, by 20-fold or greater, by 50-fold or greater, by 100-fold or greater, or even greater. e.g., as compared to a suitable control, e.g., a cell not contacted with the compound of interest. As described in greater detail below, toxicity may be reduced in a number of different ways that may depend on the particular target gene. In some instances, e.g., where the target gene includes an extended CAG repeat that results in the presence of extended polyQ domains in a product encoded by the target gene, toxicity reduction may be accompanied by a reduction in aggregation of the products encoded by the target gene. In some embodiments of the method, the protein forms aggregates in the cell and includes a polyglutamine stretch with 26 or more glutamine residues, such as 30 or more glutamine residues, 35 or more, 40 or more, 50 or more, or 60 or more glutamine residues.

In such instances, the magnitude of the reduction in aggregation may vary, and in some instances the magnitude of reduction is 2-fold or more, such as by 5-fold or more, by 10-fold or more, by 20-fold or more, by 50-fold or more, by 100-fold or more, or even more, e.g., as compared to a suitable control, e.g., a cell not contacted with the compound of interest. In some case, the magnitude of the reduction in aggregation may vary, and in some instances the magnitude of reduction is 10% or more, such as by 20% or more, by 30% or more, by 40% or more, by 50% or more, by 60% or more, by 70% or more, by 80% or more, by 90% or more, by 95% or more, by 99% or more, as compared to a suitable control, e.g., a cell not contacted with the compound of interest. Protein aggregation may be assayed using any convenient protocol, including but not limited to, the protocols described in Published United States Patent Application No. 20110130305; the disclosure of which protocols are herein incorporated by reference.

In certain embodiments, the deleterious impact or activity that is reduced by methods of the invention may be loss of function of a product encoded by the target gene. In certain of these embodiments, the wild-type or normal activity of the product encoded by the target gene is at least partially, if not completely, impaired because the target gene includes the extended trinucleotide repeat. In these instances, the loss of function is at least partially, if not completely, reversed by enhancing the desired function of the product of the target gene. The desired function of the encoded product may be enhanced by a statistically significant amount as compared to a suitable control, e.g., a cell not contacted with the compound of interest, where the magnitude of the enhancement in desired activity may be 2-fold or higher, such as 5-fold or higher, including 10-fold or higher.

In certain embodiments, the subject compounds increase the viability of the cell, as compared to a suitable control and as determined by a cell viability assay, e.g., as determined by contacting the cell with a compound of the present disclosure to a cell and determining the number of viable cells in culture using a homogeneous method, such as the CellTiter-Glo® Luminescent Cell Viability Assay.

The target gene is a gene that includes a mutant extended NR, such as a TNR, where the mutant extended nucleotide repeat domain is not present in normal versions of the gene. The term “gene” as used herein is a defined region or portion of a chromosome that encodes or enables production of a product and includes a promoter, introns, exons and enhancers. By mutant extended nucleotide repeat (NR) is meant a domain (i.e., region) of the gene that includes multiple adjacent repeats of units of 2 or more nucleotides, where a given repeating unit of nucleotides may vary in length, ranging in some instances from 2 to 10 nucleotides, such as 3 to 6 nucleotides, where examples of repeat unit lengths include units of 2 nucleotides (e.g., where the mutant extended nucleotide repeat is a dinucleotide repeat), 3 nucleotides (e.g., where the mutant extended nucleotide repeat is a trinucleotide repeat), 4 nucleotides (e.g., where the mutant extended nucleotide repeat is a tetranucleotide repeat), 5 nucleotides (e.g., where the mutant extended nucleotide repeat is a pentanucleotide repeat) or 6 nucleotides (e.g., where the mutant extended nucleotide repeat is a hexanucleotide repeat). Within a given domain, the domain may be homogeneous or heterogeneous with respect to the nature of the repeat units that make up the domain. For example, a given domain may be made up of a single type of repeat unit, i.e., al the repeat units of the domain share the same (i.e., identical) sequence of nucleotides, such that it is a homogenous mutant NR domain. Alternatively, a given domain may be made up of two or more different types of repeat units, i.e., repeat units that have differing sequences, such that it is a heterogeneous mutant NR domain. The mutant extended nucleotide repeat domain may be present in a coding or non-coding region of the target gene. In some instances, the extended nucleotide repeat domain is present in a coding region of the target gene. In some instances, the extended nucleotide repeat domain is present in a non-coding region of the target gene. The length and particular sequence of the mutant extended nucleotide repeat may vary.

In some instances, the mutant extended nucleotide repeat is a mutant extended trinucleotide repeat. By mutant extended trinucleotide repeat is meant a domain (i.e., region) of the gene that includes multiple adjacent repeats of the same three nucleotides, where the length and particular sequence of the mutant extended trinucleotide repeat may vary and the mutant extended trinucleotide repeat domain is not present in normal versions of the gene. The extended trinucleotide repeat domain may be present in a coding or non-coding region of the target gene. In some instances, the extended trinucleotide repeat domain is present in a coding region of the target gene. In some instances, the extended trinucleotide repeat domain is present in a non-coding region of the target gene. In embodiments, the mutant repeat domain is present in a non-coding region of the target gene, such as the CTG expansion located in the 3′ untranslated region of the dystrophia myotonica-protein kinase gene, which leads to Myotonic dystrophy (DM). In some instances, the mutant repeat domain is present in a coding region of the target gene, such that in some instances its presence in the target gene results in a corresponding domain or region (e.g., polyQ domain) in a product encoded by the gene. In some instances of the method, the mutant extended TNR domain is a CTG repeat domain. In certain cases, the mutant extended trinucleotide repeat domain includes 26 or more CTG repeats (e.g., 30 or more, 35 or more, etc.).

The mutant extended trinucleotide repeat may vary in terms of nucleotide composition and length. Specific trinucleotides of interest include, but are not limited to: CAG, CTG, CGG, GCC, GAA, and the like. In some instances, the mutant extended trinucleotide repeat domain is a CAG repeat domain. The particular length of the repeat domain (e.g., CAG repeat domain) may vary with the respect to the specific target gene so long as it results in deleterious activity, and in some instances is 25 repeats or longer, such as 26 repeats or longer, 30 repeats or longer, including 35 repeats or longer, 40 repeats or longer, 50 repeats or longer or even 60 repeats or longer. Specific target genes and expressed proteins of interest, diseases associated therewith and the specific length of repeat sequences of extended CAG repeats of interest, include (but are not limited to) those provided in Table 1, below.

TABLE 1

| | | disease | |
|---|---|---|---|
| | | name/protein | Pathogenic repeat |
| Disease | | product | length |
| Spinocerebellar | SCA1 | SCA1/ataxin 1 | 40~82 |
| ataxia type 1 | | | |
| Spinocerebellar | SCA2 | SCA2/ataxin 2 | 32~200 |
| ataxia type 2 | | | |
| Spinocerebellar | SCA3(MJD) | SCA3/ataxin 3 | 61~84 |
| ataxia type 3 | | | |
| Spinocerebellar | SCA7 | SCA7/ataxin 7 | 37~306 |
| ataxia type 7 | | | |
| Spinocerebellar | SCA17 | SCA17/TBP | 47~63 |
| ataxia type 17 | | | |
| Dentatorubral | DRPLA | DRPLA/atrophin 1 | 49~88 |
| pallidoluysian | | | |
| atrophy | | | |
| Spinal and bular | SBMA | Kennedy's | 38~62 |
| muscular atrophy | | disease/androgen | |
| | | receptor protein | |
| Huntington's | HD | Huntington's | 40~121 |
| disease | | Disease/huntingtin | |
| | | protein | |

The pathogenic repeat lengths shown are approximate and represent the most common range of pathogenic repeat lengths. The lower of the two numbers shown for each pathogenic repeat length indicates the length at which pathogenic effects of the expansion begin to occur. Although both cellular copies of autosomal genes responsible for NR diseases may contain NR domains, commonly one copy of the targeted gene is mutated to have an expanded NR segment, whereas the other copy (i.e., allele) contains a unexpanded NR.

As summarized above, the deleterious activity (e.g., toxicity and/or dis-functionality of products encoded thereby) of a mutant extended NR containing target gene may be reduced by the subject compounds in a variety of different ways, e.g., by reducing (and in some instances selectively reducing) the production or activity of toxic expression products (e.g., RNA or protein) encoded by the target gene, as described in greater detail below.

In some embodiments of the method, the subject compound modulates the activity of a protein encoded by the target gene. For example, with respect to polyQ repeats, in certain embodiments, the target gene is selected from genes that produce the following diseases: SCA1, SCA2, SCA3, SCA7, SCA17, DRPLA, Kennnedy's Disease and Huntington's Disease. In certain instances, the targeted disease is SCA1. In certain instances, the target disease is SCA2. In certain instances, the target disease is SCA3. In certain instances, the target disease is SCA7. In certain instances, the target disease is SCA17. In certain instances, the target disease is DRPLA. In certain instances, the target disease is Kennedy's Disease. In certain instances, the target disease is Huntington's Disease. Genes and their encoded proteins that give rise to these diseases are listed in Table 1, above. Any protein that is encoded by the target gene may be modulated, include post-translationally modified proteins. The modulated protein may be any expressed product of the gene, or a post-transcriptionally modified version thereof. In some cases, the protein is a Htt protein. In certain cases, the protein is a mutant Htt protein. Any post-translational modifications of huntingtin (Htt) proteins of interest may be modulated. Post-translational modifications of proteins of interest may regulate protein stability, localization, function, and their interactions with other molecules. Post-translational modifications may occur as chemical modifications at amino acid residues, including SUMOylation, phosphorylation, palmitoylation, acetylation, etc. Post-translational modifications may include enzymatic cleavage. Post-translational modifications may be involved in the regulation and control of a variety of cellular processes, such as Htt metabolism, protein-protein interactions and cellular toxicity.

In some instances, the subject compound modulates the functionality, e.g., binding properties, activity, etc., of the protein following expression, such that the compound is one that changes the functionality of the protein encoded by the target gene following expression of the protein from the target gene. In some cases, the compound may be one that differentially reduces the deleterious functionality, e.g., aggregation, of the encoded protein, but retains or enhances, at least to a detectable level, the beneficial activity of the encoded protein. In some cases, the compound may be one that selectively reduces the deleterious functionality, e.g., aggregation, of the encoded protein, but retains or enhances, at least to a detectable level, the beneficial activity of the encoded protein. In certain embodiments, such compounds are not inhibitors of aggregation of the protein, but instead selectively reduce the deleterious activity or functionality of the protein via another mechanism, e.g., by reducing the amount of the protein in the cell that is available for aggregation, by reducing production of a protein that is detrimental to cells independently of its propensity to aggregate, etc.

In some cases, the subject compound may change expression of a gene product, e.g., an RNA or protein. In certain embodiments of the method, the subject compound reduces the deleterious impact by modulating functionality, e.g., changing binding interactions, of a SPT4 protein in the cell. The term SPT4 protein is used herein to collectively refer to not only yeast Spt4 proteins, but also mammalian homologs thereof, e.g., human SUPT4H; murine Supt4h, etc. As such, SPT4 proteins of interest whose activity may be modulated by the selective SPT4 modulatory compounds include, but are not limited to: S. cerevisiae Spt4; human SUPT4H and murine Supt4h. The subject compounds may be referred to as SPT4 modulatory agents. SPT4 modulatory agents are compounds that change the SPT4 activity in a cell, e.g., decrease SPT4 activity in a cell. The compound may be a selective SPT4 modulatory agent. In some instances, the target SPT4 activity that is modulated, e.g., decreased, by the active compound is a transcription activity, and specifically an activity that facilitates RNA polymerase II processivity through long trinucleotide repeat domains, e.g., long CAG repeat domains. The target SPT4 activity that is modulated by such compounds is an activity arising from an SPT4 protein.

Where the subject compound employed in methods of the invention is an SPT4 modulatory agent, the compound that is employed may, upon introduction into a cell, change the SPT4 functionality in the cell, and at least differentially reduce the extended trinucleotide repeat mediated SPT4 transcription activity in the subject. The SPT4 modulatory agent may modulate functionality in a variety of ways, e.g., by inhibiting binding of an SPT4 protein to another protein, e.g., a protein interacting with SPT4 (e.g., an SPT5 protein, such as Spt5 or SUPT5H), etc. In some instances, the subject compound diminishes interaction of the SPT4 protein and a second protein. In certain instances, the second protein is a SPT5 protein. The term SPT5 protein is used herein to collectively refer to not only yeast Spt5 proteins, but also mammalian homologs thereof, e.g., human SUPT5H; murine Supt5h, etc. In certain embodiments of the method, the subject compound diminishes interaction between Supt4h and Supt5h. Human Supt4h may form a complex with Supt5h as may its yeast ortholog to regulate transcription elongation (Guo et al., “Core structure of the yeast spt4-spt5 complex: a conserved module for regulation of transcription elongation,” Structure (2008) 16: 1649-1658; Hatzog et al., “Evidence that Spt4, Spt5, and Spt6 control transcription elongation by RNA polymerase II in Saccharomyces cerevisiae,” Genes Dev. (1998) 23:357-369; Wada et al., “DSIF, a novel transcription elongation factor that regulates RNA polymerase II processivity, is composed of human Spt4 and Spt5 homologs,” Genes Dev (1998) 12: 343-356; Wenzel et al., “Crystal structure of the human transcription elongation factor DSIF hSpt4 subunit in complex with the hSpt5 dimerization interface,” Biochem J (2009) 425: 373-380). In certain embodiments of the method, the compound diminishes interaction between Supt5h and RNA polymerase II. For example, a subject compound may interfere with binding of Supt 5h to RNA polymerase II, and its effects on the interaction between Supt4h and Supt5h may be indirect.

Also provided are methods of diminishing interaction of a SPT4 protein (e.g., as described herein) and a second protein in a sample by contacting the sample with an effective amount of a compound (e.g., as described herein) that differentially, if not selectively, diminishes the interaction of the SPT4 protein and the second protein. In certain instances, the second protein is a SPT5 protein (e.g., as described herein). By “diminishes interaction” is meant that the extent of binding of the SPT4 protein to the second protein (e.g., a fraction of bound SPT4 as compared to total SPT4) is reduced by 10% or more, such as 20% or more, 30% or more, 40% or more, 50% or more, 60% or more, 70% or more, 80% or more, 90% or more, 95% or more, 99% or more, or by 100%, e.g., as compared to a suitable control, e.g., a cell not contacted with the compound of interest. Any convenient methods may be utilized to determine extent of binding of the SPT4 protein to the second protein. In certain embodiments of the method, the compound diminishes interaction between Supt4h and Supt5h. The compound may specifically bind to the SPT4 protein and disrupt the interaction of the SPT4 protein with the SPT5 protein. In some instances, the compound specifically binds to the SPT5 protein and disrupts the interaction between the SPT4 and SPT5 protein.

In some instances, an effective amount of a compound is an interaction diminishing amount, i.e., an amount of the compound that inhibits the formation of a SPT4 complex (e.g., a SPT4/SPT5 complex) by 20% or more, such as 30% or more, 40% or more, 50% or more, 60% or more, 70% or more, 80% or more, or even 90% or more, as compared to SPT4 complex formation in the absence of the compound. Any convenient methods of assaying inhibition of complex formation or competitive inhibition may be utilized, such as those methods described by Cheng et al. “Selective reduction of the deleterious activity of extended tri-nucleotide repeat containing genes” WO 2012078906, the disclosure of which assay methods are herein incorporated by reference.

Any convenient cells may be targeted for use in the subject methods. In some instances, the types of cells in which the compound exhibit activity are ones that include a target gene containing a mutant extended trinucleotide repeat. In some embodiments of the method, the cell is an animal cell or a yeast cell. In certain instances, the cell is a mammalian cell.

In practicing methods according to certain embodiments, an effective amount of the compound, e.g., SPT4 modulatory agent, is provided in the target cell or cells. In some instances, the effective amount of the compound is provided in the cell by contacting the cell with the compound. Contact of the cell with the modulatory agent may occur using any convenient protocol. The protocol may provide for in vitro or in vivo contact of the modulatory agent with the target cell, depending on the location of the target cell. In some instances, the cell is in vitro. In certain instances, the cell is in vivo. Contact may or may not include entry of the compound into the cell. For example, where the target cell is an isolated cell and the modulatory agent is an agent that modulates expression of SPT4, the modulatory agent may be introduced directly into the cell under cell culture conditions permissive of viability of the target cell. The choice of method is generally dependent on the type of cell being contacted and the nature of the compound, and the circumstances under which the transformation is taking place (e.g., in vitro, ex vivo, or in vivo).

Alternatively, where the target cell or cells are part of a multicellular organism, the modulatory agent may be administered to the organism or subject in a manner such that the compound is able to contact the target cell(s), e.g., via an in vivo or ex vivo protocol. By “in vivo,” it is meant in the target construct is administered to a living body of an animal. By “ex vivo” it is meant that cells or organs are modified outside of the body. Such cells or organs are in some cases returned to a living body.

In certain embodiments, the method is an in vivo method that includes: administering to a subject in need thereof an effective amount of a subject compound that selectively reduces the deleterious impact of the target gene to modify progression of a disease arising from the target gene in the subject. The term “treating” or “treatment” as used herein means the treating or treatment of a disease or medical condition in a patient, such as a mammal (such as a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

As used herein, the terms “host”, “subject”, “individual” and “patient” are used interchangeably and refer to any mammal in need of such treatment according to the disclosed methods. Such mammals include, e.g., humans, ovines, bovines, equines, porcines, canines, felines, non-human primate, mice, and rats. In certain embodiments, the subject is a non-human mammal. In some embodiments, the subject is a farm animal. In other embodiments, the subject is a pet. In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The amount of compound administered can be determined using any convenient methods to be an amount sufficient to produce the desired effect in association with a pharmaceutically acceptable diluent, carrier or vehicle. The specifications for the unit dosage forms of the present disclosure will depend on the particular compound employed and the effect to be achieved, and the pharmacodynamics associated with each compound in the host.

In some embodiments, an effective amount of a subject compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a subject compound is an amount that ranges from about 10 μg to about 100 mg, e.g., from about 10 μg to about 50 μg, from about 50 μg to about 150 μg, from about 150 μg to about 250 μg, from about 250 μg to about 500 μg, from about 500 μg to about 750 μg, from about 750 μg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 μg to 100 mg, or can range from 100 mg to about 500 mg, or can range from 500 mg to about 1000 mg.

In some embodiments, a single dose of the subject compound is administered. In other embodiments, multiple doses of the subject compound are administered. Where multiple doses are administered over a period of time, the RAS modulating compound is administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Any of a variety of methods can be used to determine whether a treatment method is effective. For example, a biological sample obtained from an individual who has been treated with a subject method can be assayed for the presence and/or level of cells including a mutant extended nucleotide repeat (NR) containing target gene. Assessment of the effectiveness of the methods of treatment on the subject can include assessment of the subject before, during and/or after treatment, using any convenient methods. Aspects of the subject methods further include a step of assessing the therapeutic response of the subject to the treatment.

In some embodiments, the method includes assessing the condition of the subject, including diagnosing or assessing one or more symptoms of the subject which are associated with the disease or condition of interest being treated (e.g., as described herein). In some embodiments, the method includes obtaining a biological sample from the subject and assaying the sample, e.g., for the presence of a target gene or gene product or for the presence of cells that are associated with the disease or condition of interest (e.g., as described herein). The sample can be a cellular sample. In some cases, the sample is a biopsy. The assessment step(s) of the subject method can be performed at one or more times before, during and/or after administration of the subject compounds, using any convenient methods. In certain cases, the assessment step includes identification of cells including a mutant extended nucleotide repeat (NR) containing target gene. In certain instances, assessing the subject includes diagnosing whether the subject has a disease or condition of interest.

In some instances, the method delays occurrence of a symptom associated with the disease. In certain instances, the method reduces the magnitude of a symptom associated with the disease. Disease conditions of interest include those associated with the deleterious activity of genes containing mutant extended trinucleotide repeat domains. The term “modify the progression” is employed to encompass both reduction in rate of progression (e.g., as manifested in the delay of the occurrence of one or more symptoms of the disease condition), as well as reversal of progression, including cure, of a disease condition (e.g., as manifested in the reduction of magnitude of one or more symptoms of the disease condition). In some cases, the disease or condition is a neurodegenerative disease. In certain instances, the disease or condition is a neuromuscular dysfunction disease. Specific disease conditions in which the methods and compositions of the invention find use include, but are not limited to, those listed in the Introduction section above, and include polyQ disease conditions, such as Spinocerebellar ataxia type 1, Spinocerebellar ataxia type 2, Spinocerebellar ataxia type 3, Spinocerebellar ataxia type 7, Spinocerebellar ataxia type 17, Dentatorubral pallidoluysian atrophy, spinobulbar muscular atrophy, and Huntington's Disease; other trinucleotide repeat diseases, e.g., Fragile X syndrome, Fragile XE MR, Fragile X tremor/ataxia syndrome (FXTAS), myotonic dystrophy, Friedreich's ataxia, spinocerebellar ataxia 8 (SCA8), and spinocerebellar ataxia 12 (SCA12); polyalanine expansion disorders, e.g., myotonic dystrophy type 2, spinocerebellar ataxia 10, spinocerebellar ataxia 31, progressive myoclonic epilepsy; hexanucleotide repeat disease conditions, e.g., autosomal-dominant frontotemporal dementia (FTD) and amyotrophic lateral sclerosis (ALS); and the like.

The term “surrogate marker” is employed in its conventional sense to refer to a measure of the effects of specific disease treatment or predict outcomes in a clinical trial. Surrogate markers can be defined as a laboratory measurement or a physical sign that is used in therapeutic trials as a substitute for a clinically meaningful endpoint. Reliable surrogates, rigorously validated in phase Ill clinical trials, can forecast the long term effect of the therapy based on how the patient feels, functions, or survives (Katz, “Biomarkers and Surrogate Markers: an FDA Perspective,” NeuroRx (2004) 1: 189-95). These markers may also be used to compare drug efficacy between trials and may even become the basis for which new drugs gain regulatory approval for marketing (Twaddell, “Surrogate outcome markers in research and clinical practice,” Australian Prescriber (2009) 32: 47-50). Because their use can reduce the size, duration, and cost of large studies or clinical trials, these markers are especially valuable if the predicted drug effect prevents death or promotes other critically important outcomes. For some progressive diseases, surrogate markers may be able to determine the disease stage (Weston, “The use of surrogate end points in cardiovascular disease and diabetes,” The British Journal of Cardiology (2008) 15: S6-S7). Depending on the specific disease condition, surrogate markers may vary widely. Embodiments of the present disclosure therefore include administering a compound, e.g., as described herein, to modulate, e.g., improve, one or more surrogate markers of the disease condition.

For example, where the target disease condition being treated is Huntington's Disease, a variety of different surrogate markers may be employed to monitor the disease and the effect of therapy thereon. In some instances, a surrogate marker that may evaluated includes mutant Huntingtin proteins, DNAs or RNAs and a protocol may include assaying for one or more of these markers. A protocol considered a standard method of assessing the clinical features and course of Huntington's Disease is the Unified Huntington's Disease Rating Scale (UHDRS). The method evaluates Huntington's Disease patients in four areas: motor function, cognitive function, behavioral abnormalities and functional capacity. The motor section provides a scale ranging from 0 to 4 for rating oculomotor function, dysarthria, chorea, dystonia, gait, and postural stability. A higher total score indicates more severe motor impairment. Next, a patient's cognitive function is assessed with three tests, which are a phonetic verbal fluency test, the Symbol Digit Modalities Test, and the Stroop Interference Test. Here, higher raw scores from each test indicate better cognitive performance. The behavioral portion of the protocol measures the frequency and severity of abnormalities in mood, behavior, and psychosis with a scale ranging from 0 to 4, with 0 representing an absence of a behavior and 4 representing a severe manifestation of a behavior. The total behavior score is the sum of all responses, and a higher score indicates a greater severity of behavioral symptoms. The behavioral section also prompts the evaluator to determine if the patient shows evidence of confusion, dementia, or depression. Incorporating radiographic measures of disease progression, the functional assessments include the total functional capacity score, the independence scale, and a checklist of tasks. The total functional capacity score derives from a scale ranging from 0 to 2 or 3, with 0 representing an inability to operate normally and 2 or 3 representing normal functional capacity. The independence scale ranges from 0 to 100, with each increment of 10 representing a decreased need for special care, assistance, and supervision. The checklist of questions regarding the patient's ability to carry out a task is summed by giving a score of 1 to all “yes” replies. Higher scores represent better patient functioning than lower scores (Kieburtz, et al., “Unified Huntington's Disease Rating Scale: Reliability and Consistency,” Movement Disorders (1996) 11: 136-42). Practice of embodiments of the methods results in improvement in one or more, including all of the UHDRS parameters, where the improvement in some instances is 5% or greater, such as 10% or greater, and in some instances may be 100%, or even greater.

Results from other behavioral and task completion tests may serve as surrogate markers for Huntington's Disease in embodiments of the present disclosure. The Reading the Mind in the Eyes Test (RMET), for instance, is a surrogate measure of amygdala function that is clinically useful across all disease stages in Huntington's. It is based on an individual's ability to understand the presence of beliefs, feelings, intentions and interest in other people that can differ from their own or from reality. Patients are shown a picture of the eyes and are asked to determine which of four emotional/mental state words positioned around the picture best captures the thoughts or feelings portrayed in the eyes. Performance on this test, determined by the total number of correct responses, was found to correlate negatively with proximity to disease onset and became progressively worse with each stage of disease (Mason, et al., “The role of the amygdala during emotional processing in Huntington's disease: From pre-manifest to late stage disease,” Neuropsychologia (2015) 70: 80-9). Patient speech patterns have also been analyzed for use as a marker of Huntington's Disease. Patients can be asked to read a passage or produce a monologue. Research has shown patients carrying the mutant Huntingtin (Htt) gene present with slower rates of speech, take longer to say words and produce greater silences between and within words compared to healthy individuals (Vogel, et al., “Speech acoustic markers of early stage and prodromal Huntington's disease: a marker of disease onset?,” Neurospychologia (2012) 50: 3273-8). Other markers include dual-task performance tests, where Huntington's Disease patients are slower and less accurate at performing simple tasks alone or together, and eye movements, which can provide information about disease severity and progression (Vaportzis, et al., “Effects of task difficulty during dual-task circle tracing in Huntington's disease,” Journal of Neurology (2015) 262: 268-76), (Anderson and MacAskill, “Eye movements in patients with neurodegenerative disorders,” Nature Reviews. Neurology (2013) 9: 74-85). Other markers include, but are not limited to, the Choice Reaction Task to evaluate subtle motor dysfunction, the Hopkins Verbal Learning Test to evaluate episodic memory, a computerized Mental Rotation Task to assess visuospatial processing, and a set-shifting task (Rosas, et al., “PRECREST: a phase II prevention and biomarker trial of creatine in at-risk Huntington disease,” Neurology (2014) 82: 850-7), (Beste, et al., “A novel cognitive-neurophysiological state biomarker in premanifest Huntington's disease validated on longitudinal data,” Sci. Rep. (2013) 3:1-8). Practice of embodiments of the methods can result in improvement in the parameters being measured in the particular test that is employed, where the improvement in some instances is 5% or greater, such as 10% or greater, and in some instances may be 100%, or even greater.

In some instances, samples taken from the blood, tissues and body fluids of Huntington's Disease patients are analyzed for surrogate markers. These markers may vary, where examples of such markers include analytes found in serum or physical measurements, such as pH or blood volume. The concentration, levels, or quantitative measurements of such markers in body fluids and tissues are often found to correspond with the emergence of Huntington's Disease symptoms. For example, increased serum levels of oxysterols such as free 24S-hydroxycholesterol and the 24S-hydroxycholesterol/total cholesterol ratio were associated with greater risk of impairment on tasks that assessed psychomotor speed and executive functioning. Meanwhile, higher levels of free 27-hydroxycholesterol and the 27-hydroxycholesterol/total cholesterol ratio were associated with greater risk of delayed memory impairment (Bandaru and Haughey, “Quantitative detection of free 24S-hydroxycholesterol, and 27-hydroxycholesterol from human serum,” BMC Neuroscience (2014) 15: 137). Another example of a marker found in body fluid is cortisol, of which higher concentrations in saliva was strongly associated with reduced information encoding and memory retrieval and increased motor sign severity in pre- or early-Huntington's Disease patients (Shirbin, et al., “The relationship between cortisol and verbal memory in the early stages of Huntington's Disease,” Journal of Neurology (2013) 260: 891-902). Demonstrating that physical measures may have use as surrogate markers, studies found an increase in neuronal pH and cerebral blood volume in prodromal or early-Huntington's Disease patients (Hua, et al., “Elevated arteriolar cerebral blood volume in prodromal Huntington's Disease,” Movement Disorders (2014) 29: 396-401), (Chaumeil, et al., “pH as a biomarker of neurodegeneration in Huntington's disease: a translational rodent-human MRS study,” Journal of Cerebral Blood Flow (2012) 32: 771-9). Yet another instance of a molecular surrogate is transcript expression, specifically the decrease after treatment in expression of genes that were initially expressed at higher levels in Huntington's Disease subjects compared to healthy individuals (Borovecki, et al, “Genome-wide expression profiling of human blood reveals biomarkers for Huntington's Disease,” PNAS (2005) 102: 11023-028). Other surrogate markers in body fluids include, but are not limited to: C-reactive proteins, myeloperoxidase (MPO)/white blood cell (WBC) ratio, interleukin-6 (IL-6), thioredoxin reductase-1 (TrRd-1), thioredoxin-1 (Trx-1), and muscle adenosine triphosphate (Sánchez-López, et al., “Oxidative stress and inflammation biomarkers in the blood of patients with Huntington's disease,” Neurological Research (2012) 34: 721-4), (Lodi, et al., “Abnormal in vivo skeletal muscle energy metabolism in Huntington's disease and dentatorubropallidoluysian atrophy,” Annals of Neurology (2000) 48: 72-6). Practice of embodiments of the methods can result in improvement in the marker(s) being measured in the particular test that is employed, where the improvement in some instances is 5% or greater, such as 10% or greater, and in some instances may be 100%, or even greater.

Additionally, surrogate markers for Huntington's Disease may be imaging markers, e.g., markers obtained by neuroimaging and magnetic resonance imaging (MRI). Imagining is employed to provide information about volume, levels of atrophy, and activity in white and grey matter across regions of the brain. As described by van den Bogaard et al., “MRI biomarkers in Huntington's Disease,” Frontiers in Bioscience (2012) 4: 1910-25. Common MRI methods include structural MRI, Diffusion Tensor Imaging, Magnetization Transfer Imaging, Magnetic Resonance Spectroscopy, and Functional MRI. Structural or volumetric MRI can reveal regional, progressive thinning of the cortical ribbon and grey and white matter reductions. Structural MRI scans can also detect the amount and rates of atrophy in brain regions, especially the caudate nucleus, globus pallidus, and putamen, which appears to occur in a pre- or early-disease state. Various semi- to fully-automate techniques such as Voxel Based Morphometry (VBM), Boundary Shift Integral (BSI) and FMRIB's Integrated Registration and Segmentation Technique (FIRST) have been described (van den Bogaard, et al., “MRI biomarkers in Huntington's Disease,” Frontiers in Bioscience (2012) 4: 1910-25). With Diffusion Tensor Imaging (DTI), the integrity of tissue matter is evaluated based upon the diffusion properties of protons in the intra- and extracellular space. Disturbances in fractional anisotrophy (FA), Apparent Diffusion Coefficient (ADC), mean diffusivity (MD) and total diffusivity (TraceD) in white and great matter are measured during a DTI scan. An FA value close to 0 is representative of equal diffusion in all directions. In contrast, an FA value close to or equal to 1 represents highly directional diffusion. High MD-values represent unrestricted diffusion and low MD-values suggest restricted diffusion. An increase in MD and FA values in several regions of the brain collectively demonstrated selective degeneration of connections in subcortical grey and white matter, which was likely due to the death of the striatal medium-size spiny neurons in Huntington's Disease (Douaud, et al., “In vivo evidence for the selective subcortical degeneration in Huntington's disease,” NeuroImage (2009) 46: 958-66), (van den Bogaard, et al., “MRI biomarkers in Huntington's Disease,” Frontiers in Bioscience (2012) 4: 1910-25). Another technique, Magnetization Transfer Imaging (MTI), provides a way to examine tissue structure. The technique relies on the interaction between protons in free fluid and protons bound to macromolecules. The magnetization saturation and relaxation within macromolecules affect the observable signal. The Magnetization Transfer Ratio (MTR), representing the percentage of variation in the MR signal between the saturated and unsaturated acquisitions, is a measure used in clinical studies. Two main outcome measures, the mean MTR and the MTR peak height from histogram analysis, are reported. In a study of Huntington's Disease carriers, the MTR was significantly decreased in all subcortical structures except the putamen, revealing degeneration of the subcortical and cortical grey matter (Ginestroni, et al., “Magnetization transfer MR imaging demonstrates degeneration of the subcortical and cortical gray matter in Huntington's Disease,” American Journal of Neuroradiology (2010) 31: 1807-12), (van den Bogaard, et al., “MRI biomarkers in Huntington's Disease,” Frontiers in Bioscience (2012) 4: 1910-25). Yet another technique is Magnetic Resonance Spectroscopy (MRS). MRS uses hydrogen protons to measure metabolite concentrations. Unlike previous techniques, MRS gives information about changes in physiological processes. The most common metabolites examined are: N-acetylaspertate, a marker for neuronal and axonal integrity, Creatine, a marker for brain energy metabolism, Choline, a marker reflecting membrane turnover, Myo-inositol, a marker of osmolytes and astrocytes, Lactate, a marker of interruptions of oxidative processes and the beginning of anaerobic glycolysis, and glutamate, a neurotransmitter. Decreased levels of creatine and N-acetylaspertate and increased levels of lactate across different brain regions have been reported in premanifest Huntington's disease studies (van den Bogaard, et al., “MRI biomarkers in Huntington's Disease,” Frontiers in Bioscience (2012) 4: 1910-25). Finally, functional MRI (fMRI) uses the blood-oxygen-level-dependent (BOLD) signal to discriminate brain regions with altered activation. Activation of a brain region requires an increase in energy and, consequently, blood demand, measured with fMRI. Different functional tasks such as a clock reading task, verbal working memory task, Simon task, or a porteus maze task can be employed during fMRI scanning. Abnormal connectivity or activation patterns are associated with premanifest and manifest Huntington's Disease. For instance, premanifest Huntington's Disease patients often show increased activation of several regions while there generally is a reduction of activation in premanifest gene carriers “close to onset” (van den Bogaard, et al., “MRI biomarkers in Huntington's Disease,” Frontiers in Bioscience (2012) 4: 1910-25). According to Van den Bogaard, volumetric measures and white matter diffusion tensor imaging integrity measures are the best techniques for assessing the pre-manifest stage of Huntington's disease. For early manifest Huntington's Disease, Magnetic Transfer Imaging and measurements of whole brain atrophy are more appropriate (van den Bogaard, et al., “MRI biomarkers in Huntington's Disease,” Frontiers in Bioscience (2012) 4: 1910-25). Practice of embodiments of the methods can result in improvement in the parameters being measured in the particular imaging test that is employed, where the improvement in some instances is 5% or greater, such as 10% or greater, and in some instances may be 100%, or even greater.

Separate from MRI scans, Positron Emission Tomography (PET) scans have also been employed to measure cerebral metabolic activity in premanifest Huntington's Disease patients at baseline and later in subsequent years. Metabolic brain network analysis has been increasingly used to measure the expression of characteristic spatial covariance patterns in patients experiencing neurodegeneration. Measured with [¹⁸F]-fluorodeoxyglucose scans, metabolic network activity proved sensitive to disease progression as demonstrated by its rapid rate of progression and high expression during the clinical onset of Huntington's Disease, also called phenoconversion. Abnormal elevations in baseline metabolic activity above a certain threshold indicated a high likelihood of phenoconversion in the coming years (Tang, et al., “Metabolic network as a progression biomarker of premanifest Huntington's disease,” The Journal of Clinical Investigation (2013) 123: 4076-88). A decrease in cortical glucose metabolism in the bilateral frontal, temporal and parietal cortices is also suggested as a predictor for identifying a more rapid form of disease progression in early stage Huntington's Disease patients (Shin, et al., “Decreased Metabolism in the Cerebral Cortex in Early-Stage Huntington's Disease: A Possible Biomarker of Disease Progression?,” Journal of Clinical Neurology (2013) 9: 21-5). Practice of embodiments of the methods can result in improvement in the parameters being measured in the particular imaging test that is employed, where the improvement in some instances is 5% or greater, such as 10% or greater, and in some instances may be 100%, or even greater.

Beyond body fluid based markers and imaging markers, surrogate markers for Huntington's Disease include a variety of dietary, mineral accumulation, and inclusion detection measures. One study assessed the influence of adherence to a Mediterranean diet on phenoconversion and found some correlation between high consumption of dairy products with an increased risk of higher urate levels, associated with faster progression in manifest Huntington's disease (Marder, et al., “Relationship of Mediterranean diet and caloric intake to phenoconversion in Huntington's Disease,” JAMA Neurology (2013) 70: 1382-8). In a separate study, iron accumulation was detected in the globus pallidus in both pre-Huntington's and symptomatic patients (Sánchez-Castañeda, et al., “Seeking Huntington's disease biomarkers by multimodal, cross-sectional basal ganglia imaging,” Human Brain Mapping (2013) 34: 1625-35). Another surrogate marker involves evaluation of intra-neuronal aggregates of huntingtin protein and protein fragments containing expanded polyglutamine repeats (Sieradzan, et al., “The selective vulnerability of nerve cells in Huntington's disease,” Neuropathology and Applied Neurobiology (2001) 27: 1-21), (Huang, et al., “Inducing huntingtin inclusion formation in primary neuronal cell culture and in vivo by high-capacity adenoviral vectors expressing truncated and full-length huntingtin with polyglutamine expansion,” The Journal of Gene Medicine (2008) 10: 269-79). In mice, gait analysis, immunostaining with the antibody EM48, and filter trap assays were employed together to show that early nuclear accumulation of mutant huntingtin protein or protein fragments in striatal neurons correlates with later striatal degeneration and motor deficits. Striatal phenotypes, therefore, specifically demonstrate that the disease progression is hastened by a mutant huntingtin protein fragment and may serve as surrogate markers predicting onset of Huntington's Disease (Wheeler, et al., “Early phenotypes that presage late-onset neurodegenerative disease allow testing of modifiers in Hdh CAG knock-in mice,” Human Molecular Genetics (2002) 11: 633-40). Immunostaining patterns of antibodies such as the monoclonal antibody 1C2, capable of detecting long stretches of glutamine residues, also have the potential to provide diagnostic assistance in the postmortem central nervous system analysis of Huntington's Disease (Herndon, et al., “Neuroanatomical Profile of Polyglutamine Immunoreactivity in Huntington Disease Brains,” Journal of neuropathology and experimental neurology (2009) 68: 250-61). Practice of embodiments of the methods can result in improvement in the parameters being measured in the particular test that is employed, where the improvement in some instances is 5% or greater, such as 10% or greater, and in some instances may be 100%, or even greater.

In the subject methods, the compound (e.g., as described herein) may be administered to the targeted cells using any convenient administration protocol capable of resulting in the desired activity. Thus, the subject compounds can be incorporated into a variety of formulations, e.g., pharmaceutically acceptable vehicles, for therapeutic administration. As reviewed above, the subject methods result in reduction in the deleterious activity of an extended trinucleotide repeat gene in a target cell or cells, where the target cell(s) may be in vitro or in vivo. In certain embodiments, the subject methods result in reduction in toxicity of a target gene, e.g., via a reduction in aggregation of a protein encoded thereby, in a target cell(s). In certain embodiments, the methods result in enhancement in function of a protein encoded by a target gene.

The above methods find use in a variety of different applications. Certain applications are now reviewed in the following Utility section.

### Utility

The subject methods and compound compositions find use in a variety of applications in which reduction of the deleterious activity of gene containing a mutant extended trinucleotide repeat domain is desired. As such, aspects of the invention include reducing toxicity of and/or enhancing functionality of a protein encoded by such a gene, as described herein, in any subject in need thereof, e.g., a subject that has been diagnosed with a condition that can be treated by effecting one or more of the above outcomes in the subject. Of interest is use of the subject methods and compositions to modify the progression of disease conditions associated with the deleterious activity of genes containing mutant extended trinucleotide repeat domains. The phrase “modify the progression” is employed to encompass both reduction in rate of progression (e.g., as manifested in the delay of the occurrence of one or more symptoms of the disease condition), as well as reversal of progression, including cure, of a disease condition (e.g., as manifested in the reduction of magnitude of one or more symptoms of the disease condition). Specific disease conditions in which the methods and compositions of the invention find use include, but are not limited to polyQ disease conditions, such as Spinocerebellar ataxia type 1, Spinocerebellar ataxia type 2, Spinocerebellar ataxia type 3, Spinocerebellar ataxia type 7, Spinocerebellar ataxia type 17, Dentatorubral pallidoluysian atrophy, Spinal and bular muscular atrophy, and Huntington's Disease.

In some instances, practice of subject methods results in treatment of a subject for a disease condition. By treatment is meant at least an amelioration of one or more symptoms associated with the disease condition afflicting the subject, where amelioration is used in a broad sense to refer to at least a reduction in the magnitude of a parameter, e.g., symptom, associated with the pathological condition being treated, such as loss of cognitive function, etc. As such, treatment also includes situations where the pathological condition, or at least symptoms associated therewith, are completely inhibited, e.g., prevented from happening, or stopped, e.g., terminated, such that the subject no longer suffers from the pathological condition, or at least the symptoms that characterize the pathological condition. Treatment may also manifest in the form of a modulation of a surrogate marker of the disease condition, e.g., as described above.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some embodiments, the host is human.

### Combination Therapies

The subject compounds can be administered to a subject alone or in combination with an additional, i.e., second, active agent. As such, in some cases, the subject method further comprises administering to the subject at least one additional compound. Any convenient agents may be utilized, including compounds useful for treating viral infections. The terms “agent,” “compound,” and “drug” are used interchangeably herein. For example, selective SPT4 inhibitory compounds can be administered alone or in conjunction with one or more other drugs, such as drugs employed in the treatment of polyQ diseases. In some embodiments, the method further includes coadministering concomitantly or in sequence a second agent. Possible second agents of interest include, but are not limited to, dopamine-depleting agents (e.g., tetrabenazine (Xenazine) or reserpine); dopamine-receptor antagonists (e.g., neuroleptic), amantadine, levetiracetam, anticonvulsants (e.g., valproic acid), antipsychotic drugs, such as risperidone, haloperidol (Haldol) and clozapine (Clozaril); antiseizure drugs, benzodiazepines (e.g., clonazepam (Klonopin)) and antianxiety drugs such as diazepam (Valium); antidepressants including such drugs as escitalopram (Lexapro), fluoxetine (Prozac, Sarafem) and sertraline (Zoloft); laquinimod, pridopidine, rasagiline, a pan-PPAR agonist (e.g., bezofibrate); nucleic acid silencing agents, e.g., RNA silencing agents targeting, e.g., a HTT single nucleotide polymorphism (SNP); and the like. Antisense oligonucleotides or interfering RNAs directed against SUPT4H may also be part of a combination therapy. Second active agents of interest include, but are not limited to any convenient drugs that find use against a neurodegenerative condition or disease, such as Huntington's disease.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug with a pharmaceutical composition of the present disclosure means administration of the compound and second agent at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs and compounds of the present disclosure.

In some embodiments, the compounds (e.g., a subject compound and the at least one additional compound) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

In some cases, the second active agent is a nucleoside agent. Nucleoside agents of interest include any convenient agents that reduce the deleterious activity of a mutant extended trinucleotide repeat containing target gene in a cell. As used herein, the term “nucleoside agent” is meant to include both phosphorus containing agents (e.g., nucleoside agents that include O-phosphate substituted sugar moieties) and agents that lack a phosphorus moiety. Nucleosides agent of interest may include any convenient modifications to the sugar moiety, e.g., modifications where a naturally occurring hydroxyl group is replaced with a halogen atom or an aliphatic group, or is functionalized as an ether, an amine, or the like. A nucleoside agent may contain one or more protecting groups (e.g. a hydroxyl protecting group, a bidentate diol protecting group, or a heterocyclic base protecting group) independently attached to any moiety(s) of the nucleoside agent.

Any convenient nucleoside agents may find use in the subject methods and compositions. Such nucleoside agents may be assessed, among other ways, by employing the screening methods described by Cheng et al. “Selective reduction of the deleterious activity of extended tri-nucleotide repeat containing genes” WO 2012078906, the disclosure of which screening method is herein incorporated by reference. Nucleoside agents of interest include, but are not limited to, 5-fluorouracil (5-FU), 5-FU prodrugs including tegafur and 5′-deoxyfluorouridine, fluorouridine, 2′-deoxyfluorouridine, prodrug derivatives of fluorouridine or 2′-deoxyfluorouridine, fluorocytosine, trifluoro-methyl-2′-deoxyuridine, arabinosyl cytosine, prodrugs of arabinosyl cytosine, cyclocytidine, 5-aza-2′-deoxycytidine, arabinosyl 5-azacytosine, 6-azacytidine, N-phosphonoacetyl-L-aspartic acid (PALA), pyrazofurin, 6-azauridine, azaribine, thymidine, 3-deazauridine, triacetyluridine, ethoxycarbonyluridine, triacetylcytidine, cyclocytidine, 5-aza-2′-deoxycytidine, arabinosyl 5-azacytosine, 6-azacytidine, benzylacyclouridine, benzyloxybenzylacyclouridine, aminomethyl-benzylacyclouridine, aminomethyl-benzyloxybenzylacyclouridine-, hydroxymethyl-benzylacyclouridine, hydroxymethyl-benzyloxybenzylacyclouridine, 2,2′-anhydro-5-ethyluridine, 5-benzyl barbiturate, 5-benzyloxybenzyl barbiturate, 5-benzyloxybenzyl-1-[(1-hydroxy-2-ethoxy)m-ethyl] barbiturate, 5-benzyloxybenzylacetyl-1-[(1-hydroxy-2-ethoxy)methyl] barbiturate, 5-methoxybenzylacetylacyclobarbiturate, 5-ethynyluracil, bromovinyluracil, cyanodidhydropyridine, uracil, thymine, thymidine and benzyloxybenzyluracil. Any convenient prodrugs of the subject nucleoside agents may be utilized in the subject methods. Prodrugs are frequently, although not necessarily, pharmacologically inactive until converted to the active agent. In some instances, the nucleoside agent is a ribonucleoside agent selected from a 6-deazapurine ribonucleoside and a 6-azauridine ribonucleoside, as described by Cohen et al. in WO 2016/196012, the disclosure of which is herein incorporated by reference.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

Dosage levels of the order of from about 0.01 mg to about 140 mg/kg of body weight per day are useful in representative embodiments, or alternatively about 0.5 mg to about 7 g per patient per day. Those of skill will readily appreciate that dose levels can vary as a function of the specific compound, the severity of the symptoms and the susceptibility of the subject to side effects. Dosages for a given compound are readily determinable by those of skill in the art by a variety of means.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, such as 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

As such, unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular peptidomimetic compound employed and the effect to be achieved, and the pharmacodynamics associated with each compound in the host. Those of skill in the art will readily appreciate that dose levels can vary as a function of the specific compound, the nature of the delivery vehicle, and the like. Preferred dosages for a given compound or agent are readily determinable by those of skill in the art by a variety of means.

### Kits & Systems

Also provided are kits and systems that find use in practicing embodiments of the methods, such as those described as described above. The term “system” as employed herein refers to a collection of two or more different active agents, present in a single or disparate composition, that are brought together for the purpose of practicing the subject methods. The term kit refers to a packaged active agent or agents. In some embodiments, the subject system or kit includes a dose of a subject compound (e.g., as described herein) and a dose of a second active agent (e.g., as described herein) in amounts effective to treat a subject for a disease or condition associated with the deleterious activity of a mutant extended nucleotide repeat containing target gene.

In certain instances, the second active agent is selected from: a nucleoside agent (e.g., as described herein), a dopamine-depleting agent (e.g., tetrabenazine or reserpine), a dopamine-receptor antagonist (e.g., neuroleptic), amantadine, levetiracetam, an anticonvulsant (e.g., valproic acid), a benzodiazepine agent (e.g., clonazepam), laquinimod, pridopidine, rasagiline, a pan-PPAR agonist (e.g., bezofibrate), an antipsychotic agent (e.g., risperidone or haloperidol) and a RNA silencing agent targeting a HTT single nucleotide polymorphism (SNP). Kits and systems for practicing the subject methods may include one or more pharmaceutical formulations. As such, in certain embodiments the kits may include a single pharmaceutical composition, present as one or more unit dosages, where the composition may include one or more nucleoside compounds (e.g., as described herein). In some embodiments, the kit may include two or more separate pharmaceutical compositions, each containing a different active agent, at least one of which is a nucleoside compound (e.g., as described herein).

Also of interest are kits and systems finding use in the subject methods, e.g., as described above. Such kits and systems may include one or more components of the subject methods, e.g., nucleoside agents, cells, vectors encoding proteins of interest, enzyme substrates, dyes, buffers, etc. The various kit components may be present in the containers, e.g., sterile containers, where the components may be present in the same or different containers.

In addition to the above-mentioned components, a subject kits may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXAMPLES

### Example 1: Preparation of Compounds

General Methods

¹H NMR spectra are recorded at 400 MHz on a Varian Mercury 400 spectrometer. LC-MS chromatograms and spectra were recorded on a Shimadzu LC-MS2020 using Agilent C18 column (Eclipse XDB-C18, 5 um, 2.1×50 mm) with flow rate of 1 mL/min. Mobile phase A: 0.1% of formic acid in water; mobile phase B: 0.1% of formic acid in acetonitrile. The gradient method used is:

| Time (min) | A | B |
|---|---|---|
| 0 | 95 | 5 |
| 3 | 0 | 100 |
| 4 | 0 | 100 |
| 4.05 | 95 | 5 |

Synthesis of Compounds

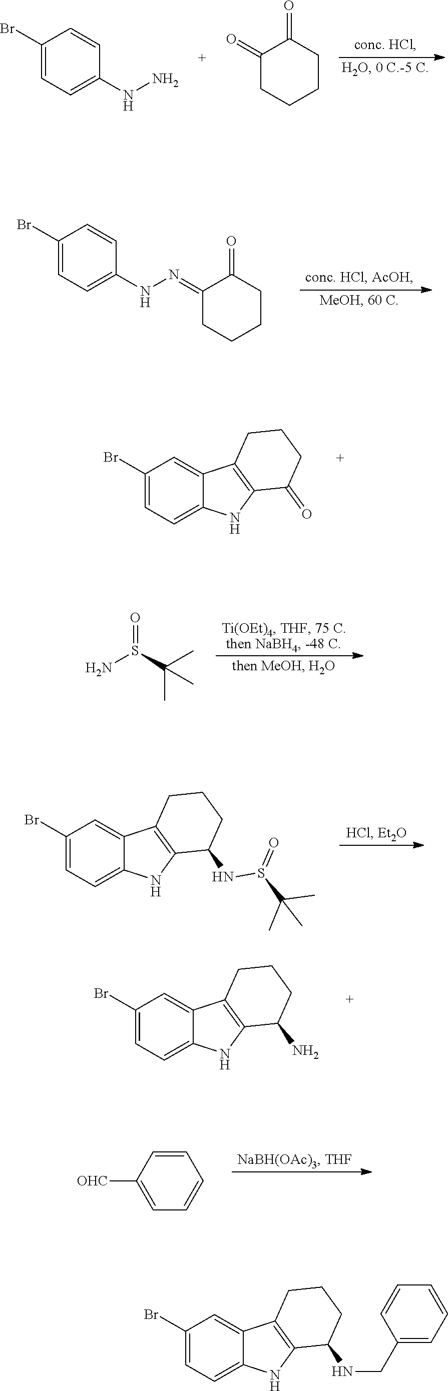

To the solution of cyclohexane-1,2-dione (2.5 g, 22.4 mmol) in water (40 mL) was at room temperature portion-wise added 4-bromophenyl)hydrazine (5.0 g, 22.4 mmol) at 0° C. Then, the reaction mixture was stirred for 16 h. The reaction was monitored by liquid chromatography-mass spectrometry and thin layer chromatography. Filtered to yield crude (E)-2-(2-(4-bromophenyl)hydrazono)cyclohexan-1-one.

To the solution of (E)-2-(2-(4-bromophenyl)hydrazono)cyclohexan-1-one (5.5 g) in methanol (20 mL), glacial acetic acid (20 mL) and concentrated hydrogen chloride (8.8 mL) was added. The mixture was heated to 60° C. for 3 h. The mixture was cooled to room temperature, filtered, and the filter cake was washed with methanol to yield 6-bromo-2,3,4,9-tetrahydro-1H-carbazol-1-one.

To the solution of 6-bromo-2,3,4,9-tetrahydro-1H-carbazol-1-one (500 mg, 1.9 mmol) in tetrahydrofuran (5 mL), was added (S)-2-methylpropane-2-sulfinamide (345 mg, 2.89 mmol) was added at ambient temperature. The reaction mixture was refluxed for 16 h at 75° C. The reaction was monitored by thin layer chromatography and liquid chromatography-mass spectrometry. The solution was cooled to ambient temperature for next step.

To the solution of sodium borohydride (289 mg, 7.6 mmol) in tetrahydrofuran (10 mL), the above reaction containing (S,E)-N-(6-bromo-2,3,4,9-tetrahydro-1H-carbazol-1-ylidene)-2-methylpropane-2-sulfinamide was added dropwise at −48° C. The reaction mixture was slowly raised to ambient temperature and stirred for 4 h. The reaction was monitored by thin layer chromatography and liquid chromatography-mass spectrometry. Then cooled to 0° C., methanol and water was added. The residue was filtered and the solid was washed with ethyl acetate. The liquid phase was concentrated, the residue was dissolved with ethyl acetate, washed with water. The organic layer was dried over anhydrous sodium sulfate, concentrated, purified by flash chromatography to yield (S)—N—((R)-6-bromo-2,3,4,9-tetrahydro-1H-carbazol-1-yl)-2-methylpropane-2-sulfinamide.

To (S)—N—((R)-6-bromo-2,3,4,9-tetrahydro-1H-carbazol-1-yl)-2-methylpropane-2-sulfinamide (1.0 g) in diethyl ether (10 ml), a solution of hydrogen chloride in diethyl ether (4 mL) was added at room temperature. The mixture was stirred for 1 h. The reaction was monitored by liquid chromatography-mass spectrometry and thin layer chromatography. The solution was concentrated and purified by recrystallization to yield (R)-6-bromo-2,3,4,9-tetrahydro-1H-carbazol-1-amine.

To a solution of (R)-6-bromo-2,3,4,9-tetrahydro-1H-carbazol-1-amine (100 mg) and benzaldehyde (42 mg) in tetrahydrofuran (10 mL) was added sodium triacetoxyborohydride at ambient temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-thin layer chromatography to yield 30 mg of (R)—N-benzyl-6-bromo-2,3,4,9-tetrahydro-1H-carbazol-1-amine as white solid.

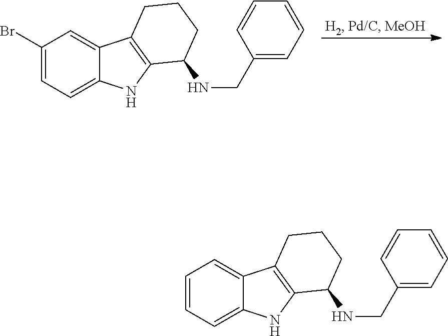 [SMILES: Brc1ccc2[nH]c3c(c2c1)CCC[C@H]3NCc1ccccc1 ; "H2, Pd/C, MeOH" ; c1ccc(CN[C@@H]2CCCc3c2[nH]c2ccccc32)cc1]

To the solution of (R)—N-benzyl-6-bromo-2,3,4,9-tetrahydro-1H-carbazol-1-amine as white solid (100 mg) in methanol (10 mL), palladium on carbon (10 mg) was added at room temperature. The mixture was stirred for 2 h under atmospheric hydrogen. The reaction was filtered, concentrated, and purified by prep-high pressure liquid chromatography to afford (R)—N-benzyl-2,3,4,9-tetrahydro-1H-carbazol-1-amine.

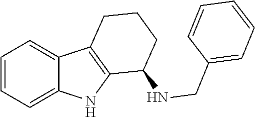 [SMILES: c1ccc(CN[C@@H]2CCCc3c2[nH]c2ccccc32)cc1]

### (R)—N-benzyl-2,3,4,9-tetrahydro-1H-carbazol-1-amine

¹H NMR (300 MHz, DMSO-d₆): δ 10.9 (s, 1H), 9.15 (broad s, 2H), 7.53 (m, 2H), 7.49 (d, J=6.0 Hz, 1H), 7.43 (m, 4H), 7.15 (t, J=6.0 Hz, 1H), 7.02 (t, J=6.0 Hz, 1H), 4.66 (broad s, 1H), 4.34 (m, 2H), 2.74 (m, 1H), 2.66 (m, 1H), 2.22 (m, 1H), 2.15 (m, 1H), 2.05 (m, 1H), 1.85 (m, 1H). Method [1] retention time 1.62 min, MS(ESI positive) 170 (M-NHBn).

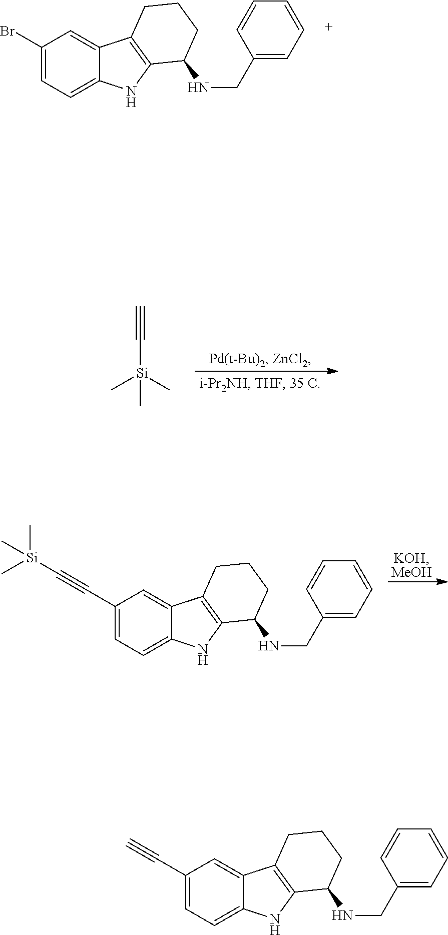

(R)—N-benzyl-6-bromo-2,3,4,9-tetrahydro-1H-carbazol-1-amine as white solid (100 mg), zinc chloride (30 mg) and bis(tri-tert-butylphosphine)palladium(0) (50 mg) was added into 50 mL three-necked round bottom. Then, nitrogen displacement three times, isopropylamine (3 mL) and tetrahydrofuran (7 mL) was added at 35 C. After 2 min, ethynyltrimethylsilane (0.2 mL) was added at 35 C. The reaction mixture was stirred for 16 h. The reaction was monitored by liquid chromatography-mass spectrometry and thin layer chromatography. Cooled to room temperature, the reaction mixture was filtered with celite. The filtration was concentrated and washed with saturated aqueous sodium bicarbonate, extracted with dichloromethane. The organic layer was dried by anhydrous sodium sulfate, concentrated, purified by prep-thin layer chromatography to yield (R)—N-benzyl-6-((trimethylsilyl)ethynyl)-2,3,4,9-tetrahydro-1H-carbazol-1-amine.

To the solution of (R)—N-benzyl-6-((trimethylsilyl)ethynyl)-2,3,4,9-tetrahydro-1H-carbazol-1-amine (30 mg) in methanol, potassium hydroxide (10 mg) was added at room temperature. The reaction was stirred for 2 h at room temperature. The reaction was monitored by liquid chromatography-mass spectrometry. Then, concentrated, dichloromethane extracted, washed with brine and water, dry over anhydrous sodium sulfate, concentrated, purified by prep-high pressure liquid chromatography to yield (R)—N-benzyl-6-ethynyl-2,3,4,9-tetrahydro-1H-carbazol-1-amine.

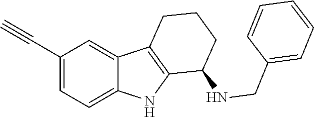 [SMILES: C#Cc1ccc2[nH]c3c(c2c1)CCC[C@H]3NCc1ccccc1]

### (R)—N-benzyl-6-ethynyl-2,3,4,9-tetrahydro-1H-carbazol-1-amine

¹H NMR (300 MHz, DMSO-d₆): δ 11.0 (s, 1H), 7.55 (s, 1H), 7.46 (m, 2H), 7.30 (m, 4H), 7.14 (d, J=6.0 Hz, 1H), 4.06 (broad m, 3H), 3.8 (s, 1H), 2.60 (m, 2H), 2.04 (m, 3H), 1.72 (m, 1H). Method [1] retention time 1.91 min, MS(ESI positive) 194 (M-NHBn), MS(ESI negative) 299 (M−H).

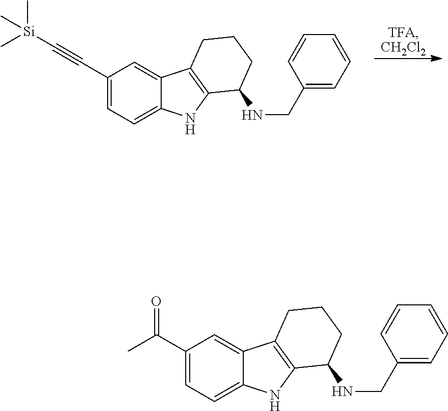

To the solution of (R)—N-benzyl-6-((trimethylsilyl)ethynyl)-2,3,4,9-tetrahydro-1H-carbazol-1-amine as white solid (30 mg) in dichloromethane, TFA (0.2 mL) was added at room temperature. The reaction was stirred for 16 h at room temperature. The reaction was monitored by liquid chromatography-mass spectrometry. Concentrated, purified by prep-high pressure liquid chromatography to yield (R)-1-(1-(benzylamino)-2,3,4,9-tetrahydro-1H-carbazol-6-yl)ethan-1-one.

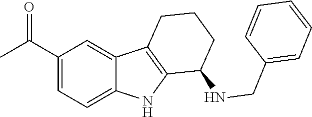 [SMILES: CC(=O)c1ccc2[nH]c3c(c2c1)CCC[C@H]3NCc1ccccc1]

### (R)-1-(1-(benzylamino)-2,3,4,9-tetrahydro-1H-carbazol-6-yl)ethan-1-one

¹H NMR (300 MHz, DMSO-d₆): δ 11.26 (s, 1H), 9.11 (broad s, 2H), 8.21 (s, 1H), 7.78 (d, J=8.0 Hz, 1H), 7.51 (m, 3H), 7.42 (m, 3H), 4.65 (broad s, 1H), 4.36 (m, 1H), 4.31 (m, 1H), 2.81 (m, 1H), 2.62 (m, 1H), 2.58 (s, 3H), 2.22 (m, 1H), 2.05 (m, 1H), 1.97 (m, 1H) 1.86 (m, 1H). Method [1] retention time 1.71 min, MS(ESI positive) 212 (M-NHBn), MS(ESI negative) 317 (M−H).

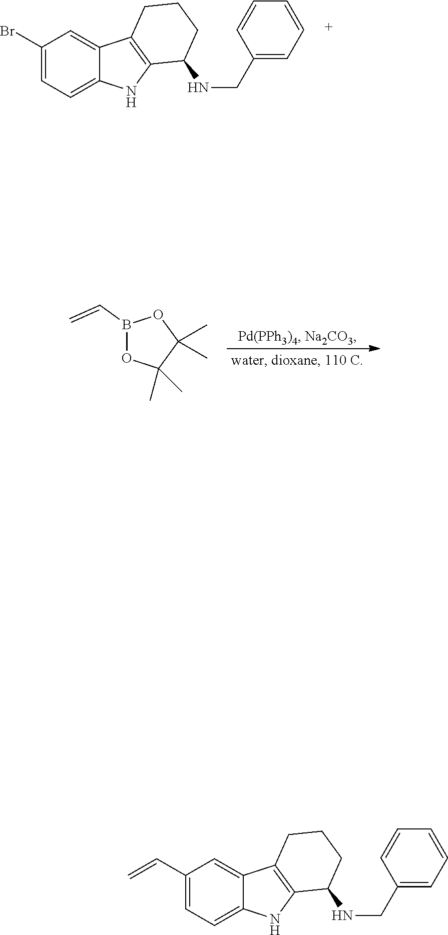 [SMILES: Brc1ccc2[nH]c3c(c2c1)CCC[C@H]3NCc1ccccc1 ; C=CB1OC(C)(C)C(C)(C)O1 ; "Pd(PPh3)4, Na2CO3," ; "water, dioxane, 110 C." ; C=Cc1ccc2[nH]c3c(c2c1)CCC[C@H]3NCc1ccccc1]

To the solution of (R)—N-benzyl-6-bromo-2,3,4,9-tetrahydro-1H-carbazol-1-amine as white solid (100 mg, 0.281 mmol) and 4,4,5,5-tetramethyl-2-vinyl-1,3,2-dioxaborolane (51.5 mmg, 0.422 mmol) in 1,4-dioxane (4 mL) and water (1 mL), sodium carbonate (59.7 mg, 0.563 mmol) and tetrakis(triphenylphosphine)palladium(0) (16.3 mg, 0.014 mmol, 0.05 eq) was added at room temperature. Then, The reaction mixture was heated to 110° C. under nitrogen atmosphere. The reaction was monitored by thin layer chromatography and liquid chromatography-mass spectrometry. Cooled to room temperature, concentrated, the residue was dissolved in ethyl acetate, washed with water dried over anhydrous sodium sulfate, concentrated and purified to yield (R)—N-benzyl-6-vinyl-2,3,4,9-tetrahydro-1H-carbazol-1-amine.

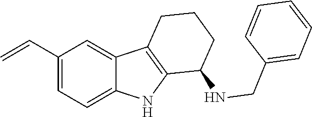 [SMILES: C=Cc1ccc2[nH]c3c(c2c1)CCC[C@H]3NCc1ccccc1]

### (R)—N-benzyl-6-vinyl-2,3,4,9-tetrahydro-1H-carbazol-1-amine

¹H NMR (300 MHz, DMSO-d₆): δ 10.94 (s, 1H), 9.13 (broad s, 2H), 7.52 (broad s, 3H), 7.35 (m, 5H), 6.77 (dd, J=15 and 9 Hz, 1H), 5.68 (d, J=15 Hz, 1H), 5.08 (d, J=9.0 Hz, 1H), 4.62 (broad s, 1H), 4.34 (m, 1H), 4.29 (m, 1H), 2.73 (m, 1H), 2.62 (m, 1H), 2.19 (m, 1H), 2.11 (m, 1H), 2.04 (m, 1H), 1.83 (m, 1H). Method [1] retention time 1.89 min, MS(ESI positive) 196 (M-NHBn).

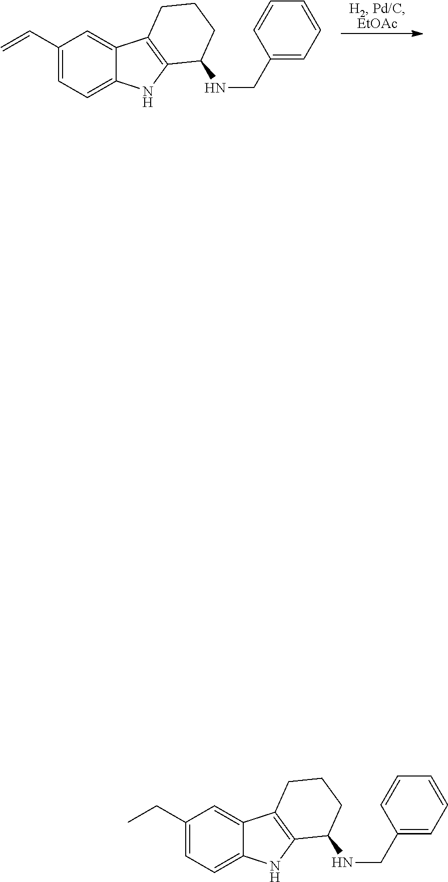 [SMILES: C=Cc1ccc2[nH]c3c(c2c1)CCC[C@H]3NCc1ccccc1 ; "H2, Pd/C,EtOAc" ; CCc1ccc2[nH]c3c(c2c1)CCC[C@H]3NCc1ccccc1]

To a solution of (R)—N-benzyl-6-vinyl-2,3,4,9-tetrahydro-1H-carbazol-1-amine as white solid (80 mg) in ethyl acetate (40 mL), palladium on carbon (25 mg) was added. Then hydrogen displacement three times and stirred for 2 h. The reaction was monitored by high pressure liquid chromatography and 1H NMR. Filtered, the filtration was concentrated for the crude product and purified by prep-high pressure liquid chromatography to afford (R)—N-benzyl-6-ethyl-2,3,4,9-tetrahydro-1H-carbazol-1-amine.

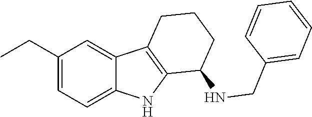 [SMILES: CCc1ccc2[nH]c3c(c2c1)CCC[C@H]3NCc1ccccc1]

### (R)—N-benzyl-6-ethyl-2,3,4,9-tetrahydro-1H-carbazol-1-amine

¹H NMR (300 MHz, DMSO-d₆): δ 10.68 (broad s, 1H), 9.02 (broad s, 2H), 7.52 (d, J=6.0 Hz, 2H), 7.42 (m, 3H), 7.32 (d, J=6.0 Hz, 1H), 7.27 (s, 1H), 7.00 (d, J=6.0 Hz, 1H), 4.61 (broad s, 1H), 4.33 (m, 2H), 2.65 (m, 4H), 2.18 (m, 1H), 2.12 (m, 1H), 2.00 (m, 1H), 1.22 (m, 1H) 1.17 (t, J=8.0 Hz, 3H). Method [1] retention time 2.00 min, MS(ESI positive) 198 (M-NHBn).

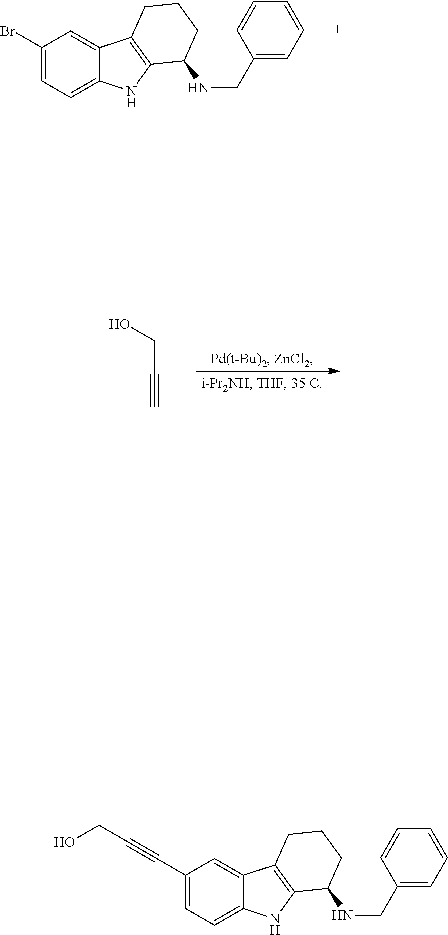

(R)—N-benzyl-6-bromo-2,3,4,9-tetrahydro-1H-carbazol-1-amine (100 mg), zinc chloride (30 mg) and bis(tri-tert-butylphosphine)palladium(0) (50 mg) was added into a 50 mL three-necked round bottom. Then, nitrogen displacement three times, isopropylamine (3 mL) and tetrahydrofuran (7 mL) was added at 35 C. After 2 min, prop-2-yn-1-ol (0.2 mL) was added at 35 C. The reaction mixture was stirred for 16 h. The reaction was monitored by liquid chromatography-mass spectrometry and thin layer chromatography. Cooled to room temperature, the reaction mixture was filtered with celite. The filtration was concentrated and washed with saturated aqueous sodium bicarbonate, extracted with dichloromethane. The organic layer was dried by anhydrous sodium sulfate, concentrated, purified by prep-thin layer chromatography to yield 5 mg of (R)-3-(1-(benzylamino)-2,3,4,9-tetrahydro-1H-carbazol-6-yl)prop-2-yn-1-ol.

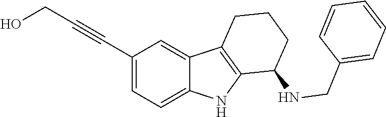 [SMILES: OCC#Cc1ccc2[nH]c3c(c2c1)CCC[C@H]3NCc1ccccc1]

(R)-3-(1-(benzylamino)-2,3,4,9-tetrahydro-1H-carbazol-6-yl)prop-2-yn-1-ol ¹H NMR (300 MHz, DMSO-d₆): δ 11.10 (broad s, 1H), 9.10 (broad s, 2H), 7.59 (s, 1H), 7.51 (m, 2H), 7.42 (m, 4H), 7.17 (dd, J=6.3 and 1.2 Hz, 1H), 4.63 (broad s, 1H), 4.37 (m, 1H), 4.30 (m, 1H), 4.24 (s, 2H), 2.74 (m, 1H), 2.62 (m, 1H), 2.19 (m, 1H), 2.10 (m, 1H), 2.01 (m, 1H) 1.80 (m, 1H). Method [1] retention time 1.71 min, MS(ESI positive) 224 (M-NHBn).

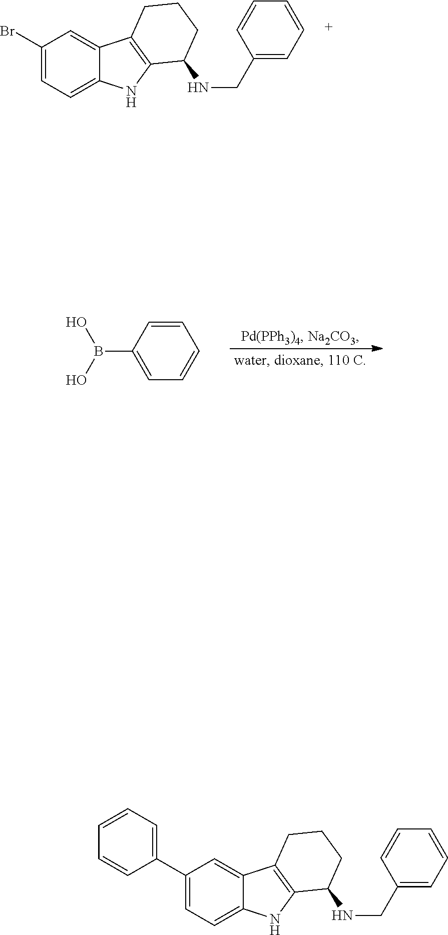 [SMILES: Brc1ccc2[nH]c3c(c2c1)CCC[C@H]3NCc1ccccc1 ; OB(O)c1ccccc1 ; "Pd(PPh3)4, Na2CO3," ; "water, dioxane, 110 C." ; c1ccc(CN[C@@H]2CCCc3c2[nH]c2ccc(-c4ccccc4)cc32)cc1]

To the solution of (R)—N-benzyl-6-bromo-2,3,4,9-tetrahydro-1H-carbazol-1-amine as white solid (100 mg, 0.281 mmol) and phenylboronic acid (51.5 mg, 0.422 mmol, 1.5 eq) in 1,4-dioxane (4 mL) and water 1 (mL), sodium carbonate (59.7 mg, 0.563 mmol) and tetrakis(triphenylphosphine)palladium(0) (16.3 mg, 0.014 mmol) was added at room temperature. Then, the reaction mixture was heated to 110° C. under nitrogen atmosphere. The reaction was monitored by thin layer chromatography and liquid chromatography-mass spectrometry. Cooled to room temperature, concentrated, the residue was dissolved in ethyl acetate and washed with water, dried over anhydrous sodium sulfate, concentrated, and purified to yield (R)—N-benzyl-6-phenyl-2,3,4,9-tetrahydro-1H-carbazol-1-amine.

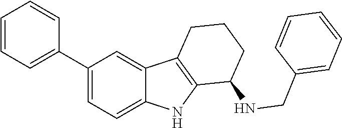 [SMILES: c1ccc(CN[C@@H]2CCCc3c2[nH]c2ccc(-c4ccccc4)cc32)cc1]

### (R)—N-benzyl-6-phenyl-2,3,4,9-tetrahydro-1H-carbazol-1-amine

¹H NMR (300 MHz, CD₃OD): δ 7.54 (s, 1H), 7.63 (d, J=6.0 Hz, 2H), 7.53 (m, 2H), 7.47 (m, 5H), 7.41 (t, J=6.0 Hz, 2H), 7.27 (t, J=6.0 Hz, 1H), 4.69 (m, 1H), 4.44 (broad s, 2H), 2.96 (m, 1H), 2.83 (m, 1H), 2.32 (m, 2H), 2.11 (m, 1H), 2.05 (m, 1H). Method [1] retention time 2.00 min, MS(ESI positive) 246 (M-NHBn), MS(ESI negative) 351 (M−H).

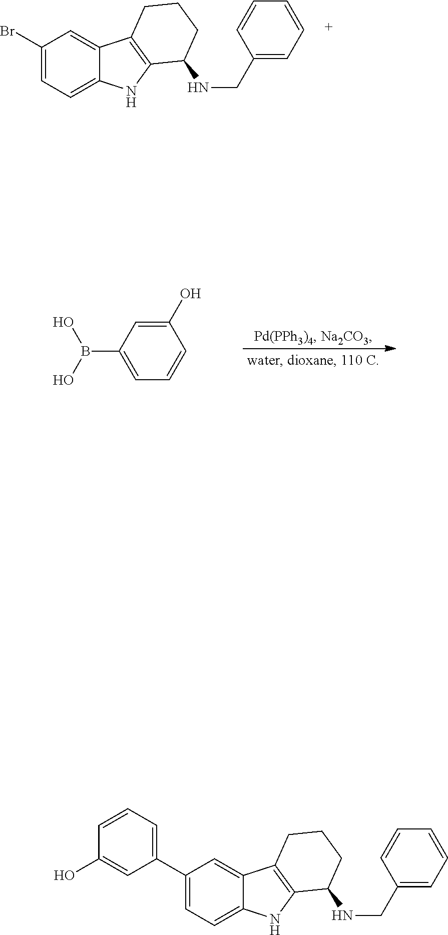 [SMILES: Brc1ccc2[nH]c3c(c2c1)CCC[C@H]3NCc1ccccc1 ; OB(O)c1cccc(O)c1 ; "Pd(PPh3)4, Na2CO3," ; "water, dioxane, 110 C." ; Oc1cccc(-c2ccc3[nH]c4c(c3c2)CCC[C@H]4NCc2ccccc2)c1]

To the solution of (R)—N-benzyl-6-bromo-2,3,4,9-tetrahydro-1H-carbazol-1-amine as white solid (100 mg, 0.281 mmol) and (3-hydroxyphenyl)boronic acid (51.5 mmg, 0.422 mmol) in 1,4-dioxane (4 mL) and water (1 mL), sodium carbonate (59.7 mg, 0.563 mmol) and tetrakis(triphenylphosphine)palladium(0) (16.3 mg, 0.014 mmol) was added at room temperature. Then, the reaction mixture was heated to 110° C. under nitrogen atmosphere. The reaction was monitored by thin layer chromatography and liquid chromatography-mass spectrometry. Cooled to room temperature, concentrated, the residue was dissolved in ethyl acetate, washed with water, dried over anhydrous sodium sulfate, concentrated, and purified to yield (R)-3-(1-(benzylamino)-2,3,4,9-tetrahydro-1H-carbazol-6-yl)phenol.

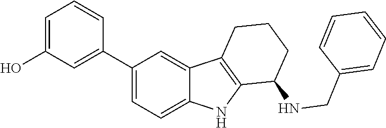 [SMILES: Oc1cccc(-c2ccc3[nH]c4c(c3c2)CCC[C@H]4NCc2ccccc2)c1]

### (R)-3-(1-(benzylamino)-2,3,4,9-tetrahydro-1H-carbazol-6-yl)phenol

¹H NMR (300 MHz, DMSO-d₆): δ 11.10 (d, J=2.4 Hz, 1H), 9.39 (broad s, 1H), 9.11 (broad s, 2H), 7.63 (s, 1H), 7.52 (m, 2H), 7.43 (m, 7H), 6.80 (d, J=6.6 Hz, 2H), 4.64 (broad s, 1H), 4.30 (m, 2H), 2.78 (m, 1H), 2.66 (m, 1H), 2.19-2.09 (m, 3H), 1.83 (m, 1H). Method [1] retention time 1.86 min, MS(ESI positive) 262 (M-NHBn), MS(ESI negative) 367 (M−H).

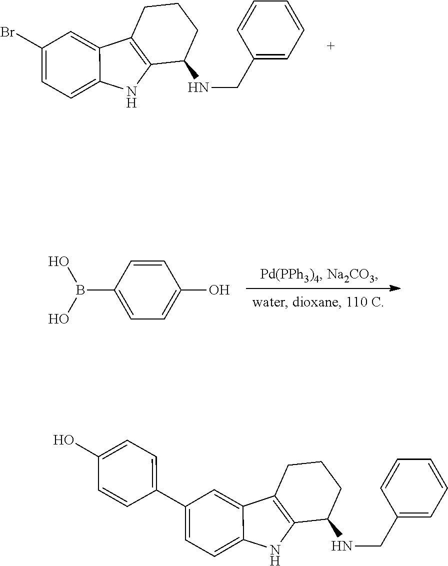 [SMILES: Brc1ccc2[nH]c3c(c2c1)CCC[C@H]3NCc1ccccc1 ; OB(O)c1ccc(O)cc1 ; Oc1ccc(-c2ccc3[nH]c4c(c3c2)CCC[C@H]4NCc2ccccc2)cc1]

To the solution of (R)—N-benzyl-6-bromo-2,3,4,9-tetrahydro-1H-carbazol-1-amine as white solid (100 mg, 0.281 mmol) and (4-hydroxyphenyl)boronic acid (51.5 mmg, 0.422 mmol) in 1,4-dioxane (4 mL) and water (1 mL), sodium carbonate (59.7 mg, 0.563 mmol) and tetrakis(triphenylphosphine)palladium(0) (16.3 mg, 0.014 mmol) was added at room temperature. Then, the reaction mixture was heated to 110° C. under nitrogen atmosphere. The reaction was monitored by thin layer chromatography and liquid chromatography-mass spectrometry. Cooled to room temperature, concentrated, the residue was dissolved in ethyl acetate, washed with water, dried over anhydrous sodium sulfate, concentrated, and purified to yield (R)-4-(1-(benzylamino)-2,3,4,9-tetrahydro-1H-carbazol-6-yl)phenol.

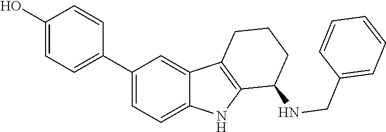 [SMILES: Oc1ccc(-c2ccc3[nH]c4c(c3c2)CCC[C@H]4NCc2ccccc2)cc1]

### (R)-4-(1-(benzylamino)-2,3,4,9-tetrahydro-1H-carbazol-6-yl)phenol

¹H NMR (300 MHz, DMSO-d₆): δ 10.86 (broad s, 1H), 9.39 (broad s, 1H), 9.11 (broad s, 2H), 7.63 (s, 1H), 7.52 (m, 2H), 7.41 (m, 7H), 6.80 (d, J=6.0 Hz, 2H), 4.64 (broad s, 1H), 4.31 (m, 2H), 2.79 (m, 1H), 2.66 (m, 1H), 2.21-2.04 (m, 3H), 1.81 (m, 1H). Method [1] retention time 1.81 min, MS(ESI positive) 262 (M-NHBn), MS(ESI negative) 367 (M−H).

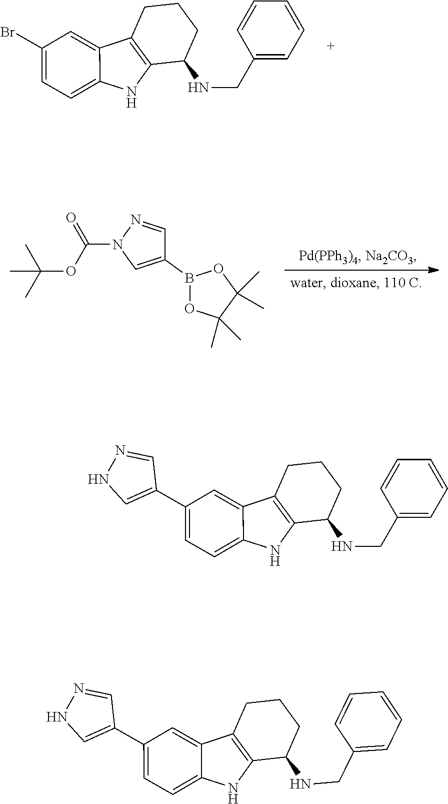 [SMILES: Brc1ccc2[nH]c3c(c2c1)CCC[C@H]3NCc1ccccc1 ; CC(C)(C)OC(=O)n1cc(B2OC(C)(C)C(C)(C)O2)cn1 ; c1ccc(CN[C@@H]2CCCc3c2[nH]c2ccc(-c4cn[nH]c4)cc32)cc1 ; c1ccc(CN[C@@H]2CCCc3c2[nH]c2ccc(-c4cn[nH]c4)cc32)cc1]

### (R)—N-benzyl-6-(1H-pyrazol-4-yl)-2,3,4,9-tetrahydro-1H-carbazol-1-amine

¹H NMR (300 MHz, DMSO-d₆): δ 10.81 (m, J=4.2 Hz, 1H), 9.11 (broad s, 2H), 7.97 (s, 2H), 7.68 (s, 1H), 7.52 (m, 2H), 7.41 (m, 5H), 4.63 (broad s, 1H), 4.31 (m, 2H), 2.75 (m, 1H), 2.67 (m, 1H), 2.20-2.02 (m, 3H), 1.84 (m, 1H). Method [1] retention time 1.65 min, MS(ESI positive) 236 (M-NHBn), MS(ESI negative) 341 (M−H).

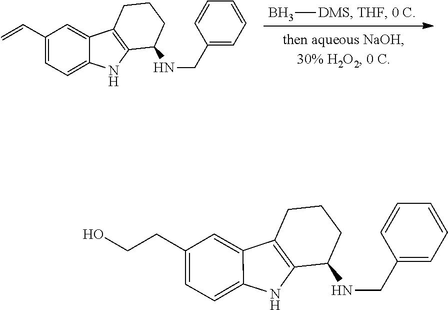

To a solution of (R)—N-benzyl-6-vinyl-2,3,4,9-tetrahydro-1H-carbazol-1-amine as white solid (150 mg) in tetrahydrofuran (20 mL), was added 2M borane-dimethylsulfide (1.5 mL) at OC. The mixture was stirred for 1 h. Then 2.5M aqueous sodium hydroxide (0.75 mL) was added at OC. 30% aqueous H₂O₂ (0.9 mL) was added at OC. The mixture was stirred for another 2 h. The reaction was monitored by thin layer chromatography and liquid chromatography-mass spectrometry. The reaction was quenched with aqueous sodium thiosulfate, ethyl acetate extracted, dry over anhydrous sodium sulfate, concentrated, purified by prep-thin layer chromatography and prep-high pressure liquid chromatography to yield (R)-2-(1-(benzylamino)-2,3,4,9-tetrahydro-1H-carbazol-6-yl)ethan-1-ol.

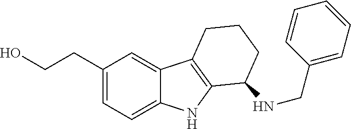 [SMILES: OCCc1ccc2[nH]c3c(c2c1)CCC[C@H]3NCc1ccccc1]

### (R)-2-(1-(benzylamino)-2,3,4,9-tetrahydro-1H-carbazol-6-yl)ethan-1-ol

¹H NMR (300 MHz, CD₃OD): δ 7.50 (m, 2H), 7.44 (m, 3H), 7.35 (s, 1H), 7.31 (d, J=6.0 Hz, 1H), 7.07 (t, J=6.0 Hz, 1H), 4.64 (m, 1H), 4.39 (s, 2H), 3.74 (t, J=5.4 Hz, 2H), 2.82 (m, 3H), 2.75 (m, 1H), 2.27 (m, 2H), 2.05 (m, 1H), 1.96 (m, 1H). Method [1] retention time 1.62 min, MS(ESI positive) 214 (M-NHBn), MS(ESI negative) 319 (M−H).

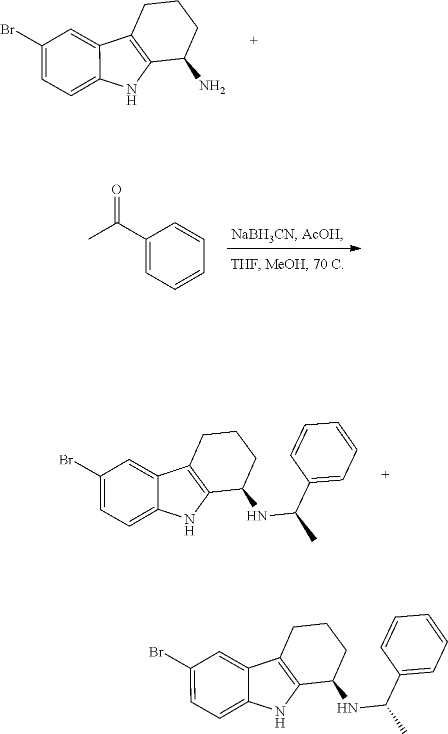

To a solution of (R)-6-bromo-2,3,4,9-tetrahydro-1H-carbazol-1-amine (100 mg) and acetophenone (50 mg) in methanol (2 ml) and tetrahydrofuran (6 ml), was added a sodium cyanoborohydride (72 mg) and catalytic glacial acetic acid at room temperature. Then, the mixture was stirred for 16 h at 70 C. The reaction was monitored by thin layer chromatography and liquid chromatography-mass spectrometry. Cooled to room temperature, poured into saturated aqueous sodium bicarbonate, ethyl acetate extracted, washed with brine, dry over anhydrous sodium sulfate, concentrated purified by prep-high pressure liquid chromatography to yield 50 mg of (R)-6-bromo-N—((R)-1-phenylethyl)-2,3,4,9-tetrahydro-1H-carbazol-1-amine and 50 mg of (R)-6-bromo-N—((S)-1-phenylethyl)-2,3,4,9-tetrahydro-1H-carbazol-1-amine.

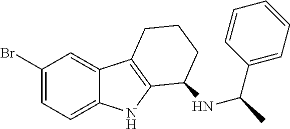 [SMILES: C[C@@H](N[C@@H]1CCCc2c1[nH]c1ccc(Br)cc21)c1ccccc1]

### (R)-6-bromo-N—((R)-1-phenylethyl)-2,3,4,9-tetrahydro-1H-carbazol-1-amine (R)

Stereochemistry at Benzylic Position Arbitrarily Assigned

¹H NMR (300 MHz, DMSO-d₆): δ 10.81 (broad s, 1H), 9.27 (broad s, 1H), 9.07 (broad s, 1H), 7.65 (m, 3H), 7.44 (m, 4H), 7.25 (d, J=6.0 Hz, 1H), 4.71 (q, J=6.0 Hz, 1H), 4.34 (m, 1H), 2.71 (m, 1H), 2.58 (m, 1H), 1.92 (m, 3H), 1.84 (m, 1H), 1.58 (d, J=6.0 Hz, 3H).

Method [1] retention time 1.98 min, MS(ESI positive) 248 and 250 (M-NHCH(Me)Ph), MS(ESI negative) 367 and 369 (M−H).

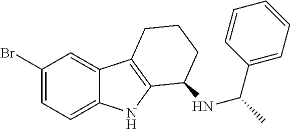 [SMILES: C[C@H](N[C@@H]1CCCc2c1[nH]c1ccc(Br)cc21)c1ccccc1]

### (R)-6-bromo-N—((S)-1-phenylethyl)-2,3,4,9-tetrahydro-1H-carbazol-1-amine (S)

Stereochemistry at Benzylic Position Arbitrarily Assigned

¹H NMR (300 MHz, DMSO-d₆): δ 11.17 (broad s, 1H), 9.31 (broad s, 2H), 7.65 (s, 1H), 7.59 (d, J=6.0 Hz, 2H), 7.42 (m, 4H), 7.24 (d, J=6.0 Hz, 1H), 4.71 (m, 1H), 4.22 (m, 1H), 2.68 (m, 1H), 2.58 (m, 1H), 2.17 (m, 1H), 1.97 (m, 2H), 1.74 (m, 1H), 1.61 (d, J=6.0 Hz, 3H). Method [1] retention time 2.01 min, MS(ESI positive) 248 and 250 (M-NHCH(Me)Ph), MS(ESI negative) 367 and 369 (M−H).

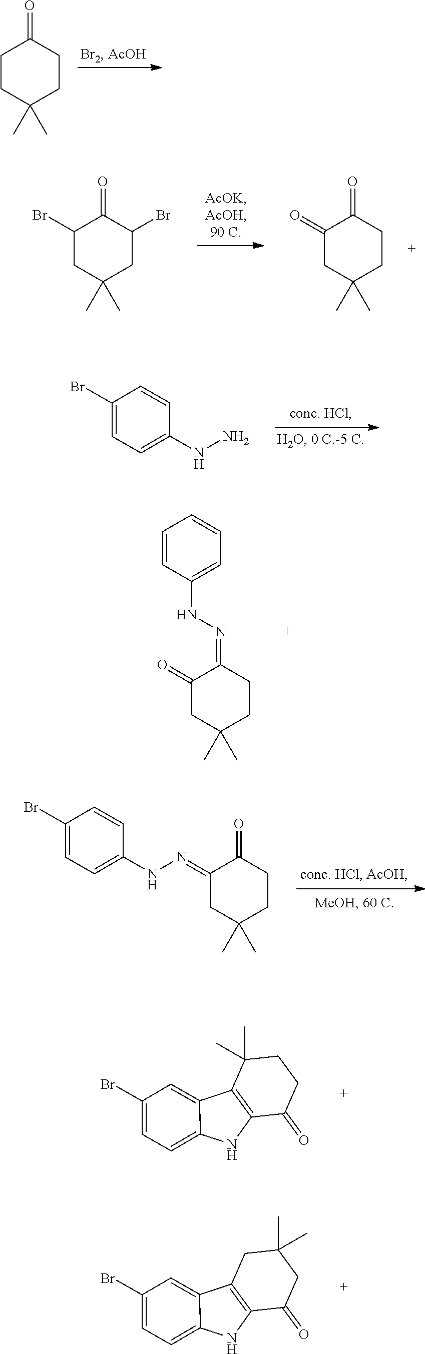

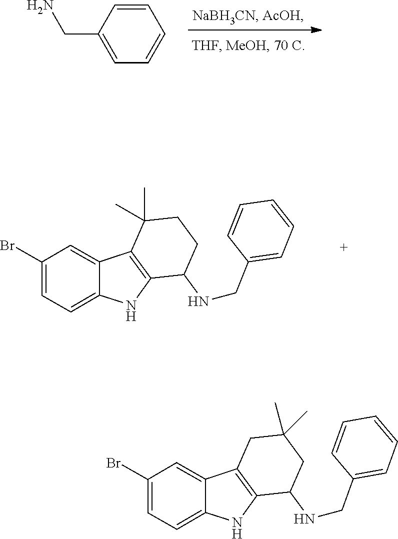 [SMILES: NCc1ccccc1 ; CC1(C)CCC(NCc2ccccc2)c2[nH]c3ccc(Br)cc3c21 ; CC1(C)Cc2c([nH]c3ccc(Br)cc23)C(NCc2ccccc2)C1]

To a solution of 4,4-dimethylcyclohexan-1-one in glacial acetic acid (5 mL), was added bromine (2.54 g, 15.9 mmol) was added room temperature. The reaction was stirred for 2 h. The reaction was concentrated. The residue was added into water and ether. Extracted with diethyl ether and washed with saturated aqueous sodium bicarbonate, dried over anhydrous sodium sulfate. Concentrated for the crude as yellow oil. Recrystallization with hexane to yield 900 mg of 2,6-dibromo-4,4-dimethylcyclohexan-1-one.

To a solution of 2,6-dibromo-4,4-dimethylcyclohexan-1-one (5.0 g, 17.7 mmol) in glacial acetic acid (50 mL), was added potassium acetate (8.3 g, 84.6 mmol) at room temperature. The mixture was heated to 90° C. and stirred for 2 h. The reaction was monitored by liquid chromatography-mass spectrometry. The reaction solution was cooled to room temperature and poured into water 300 mL. Extracted with methyl tert-butyl ether (200 mL) twice, washed with saturated aqueous sodium bicarbonate and 10% aqueous sodium hydroxide. The aqueous phase was acid with con. H₂SO₄ and extracted with methyl tert-butyl ether (200 mL) twice and dried over anhydrous sodium sulfate, concentrated for the crude product and purified by flash column chromatography to yield 420 mg of 4,4-dimethylcyclohexane-1,2-dione as a white solid.

To a turbid liquid of 4,4-dimethylcyclohexane-1,2-dione (200 mg) in concentrated hydrogen chloride (4 mL), 4-bromophenyl)hydrazine (319 mg) was added at room temperature. The mixture was stirred for 16 h at room temperature. The reaction was monitored by liquid chromatography-mass spectrometry. Filtered, the yellow solid was washed with water for the crude product 180 mg of (E)-2-(2-(4-bromophenyl)hydrazono)-4,4-dimethylcyclohexan-1-one and (Z)-5,5-dimethyl-2-(2-phenylhydrazono)cyclohexan-1-one.

To a solution of (E)-2-(2-(4-bromophenyl)hydrazono)-4,4-dimethylcyclohexan-1-one and (Z)-5,5-dimethyl-2-(2-phenylhydrazono)cyclohexan-1-one (2.5 g) in methanol (20 mL), was added glacial acetic acid (20 mL) and concentrated hydrogen chloride (10 mL) at room temperature. The reaction mixture was stirred for 3 h at 60 C. Monitored by thin layer chromatography and liquid chromatography-mass spectrometry. Cooled to room temperature, filtered for the crude product as brown solid. The solid was washed with water, and triturated with methanol at 70 C. Then, cooled to room temperature filtered to yield 600 mg of 6-bromo-3,3-dimethyl-2,3,4,9-tetrahydro-1H-carbazol-1-one. The filtrate was concentrated, ethyl acetate extracted and washed with water, purified by flash column chromatography for 400 mg crude. Then purified by prep-thin layer chromatography to yield 6-bromo-4,4-dimethyl-2,3,4,9-tetrahydro-1H-carbazol-1-one.

To a solution of 6-bromo-3,3-dimethyl-2,3,4,9-tetrahydro-1H-carbazol-1-one (100 mg) and phenylmethanamine (146 mg) in 8 mL (methanol/tetrahydrofuran=1:3), was added a sodium cyanoborohydride (130 mg) and catalytic glacial acetic acid at room temperature. Then, the mixture was stirred for 16 h at 70 C. The reaction was monitored by thin layer chromatography and liquid chromatography-mass spectrometry and purified to yield N-benzyl-6-bromo-3,3-dimethyl-2,3,4,9-tetrahydro-1H-carbazol-1-amine.

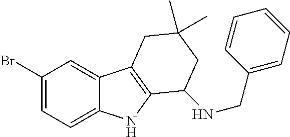 [SMILES: CC1(C)Cc2c([nH]c3ccc(Br)cc23)C(NCc2ccccc2)C1]

### N-benzyl-6-bromo-3,3-dimethyl-2,3,4,9-tetrahydro-1H-carbazol-1-amine

¹H NMR (300 MHz, DMSO-d₆): δ 11.03 (d, J=6.0 Hz, 1H), 9.62 (broad s, 1H), 9.26 (broad s, 1H), 7.63 (s, 1H), 7.51 (d, J=6.0 Hz, 2H), 7.42 (m, 4H), 7.23 (d, J=6.0 Hz, 1H), 4.70 (broad s, 1H), 4.31 (m, 1H), 4.25 (m, 1H), 2.51 (m, 2H), 2.13 (m, 1H), 1.85 (t, J=6.0 Hz, 1H), 1.19 (s, 3H), 0.86 (s, 3H). Method [1] retention time 2.08 min, MS(ESI positive) 276 and 278 (M-NHBn), MS(ESI negative) 381 and 383 (M−H).

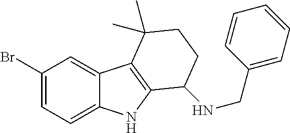 [SMILES: CC1(C)CCC(NCc2ccccc2)c2[nH]c3ccc(Br)cc3c21]

### N-benzyl-6-bromo-4,4-dimethyl-2,3,4,9-tetrahydro-1H-carbazol-1-amine

¹H NMR (300 MHz, DMSO-d₆): δ 11.12 (m, 1H), 9.09 (broad s, 2H), 7.81 (s, 1H), 7.53 (d, J=6.0 Hz, 2H), 7.41 (m, 4H), 7.24 (t, J=6.0 Hz, 1H), 4.56 (broad s, 1H), 4.56 (m, 1H), 4.28 (m, 1H), 2.16 (m, 2H), 1.89 (m, 1H), 1.57 (m, 1H), 1.41 (s, 3H), 1.28 (s, 3H). Method [1] retention time 1.74 min, MS(ESI positive) 276 and 278 (M-NHBn), MS(ESI negative) 381 and 383 (M−H).

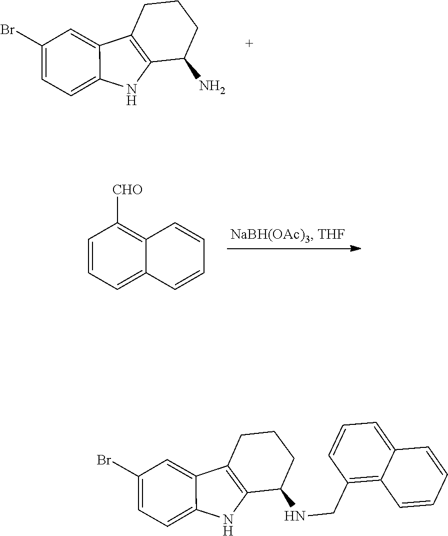 [SMILES: N[C@@H]1CCCc2c1[nH]c1ccc(Br)cc21 ; O=Cc1cccc2ccccc12 ; Brc1ccc2[nH]c3c(c2c1)CCC[C@H]3NCc1cccc2ccccc12]

To a solution of (R)-6-bromo-2,3,4,9-tetrahydro-1H-carbazol-1-amine (100 mg) and 1-naphthaldehyde (71 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 112 mg of (R)-6-bromo-N-(naphthalen-1-ylmethyl)-2,3,4,9-tetrahydro-1H-carbazol-1-amine.

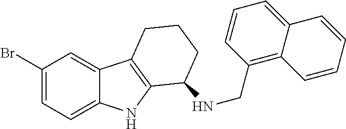 [SMILES: Brc1ccc2[nH]c3c(c2c1)CCC[C@H]3NCc1cccc2ccccc12]

### (R)-6-bromo-N-(naphthalen-1-ylmethyl)-2,3,4,9-tetrahydro-1H-carbazol-1-amine

¹H NMR (300 MHz, DMSO-d₆): δ 11.22 (s, 1H), 9.22 (m, 2H), 8.18 (d, J=6.0 Hz, 1H), 8.08 (d, J=6.0 Hz, 2H), 7.60 (d, J=6.0 Hz, 2H), 7.55 (m, 3H), 7.41 (d, J=6.0 Hz, 1H), 7.25 (d, J=6.0 Hz, 1H), 4.88 (broad s, 2H), 4.77 (m, 1H), 2.74 (m, 1H), 2.65 (m, 1H), 2.21 (m, 1H), 2.15 (m, 1H), 2.07 (m, 1H), 1.85 (m, 1H). Method [1] retention time 2.06 min, MS(ESI positive) 248 and 250 (M-NHBn), MS(ESI negative) 403 and 405 (M−H).

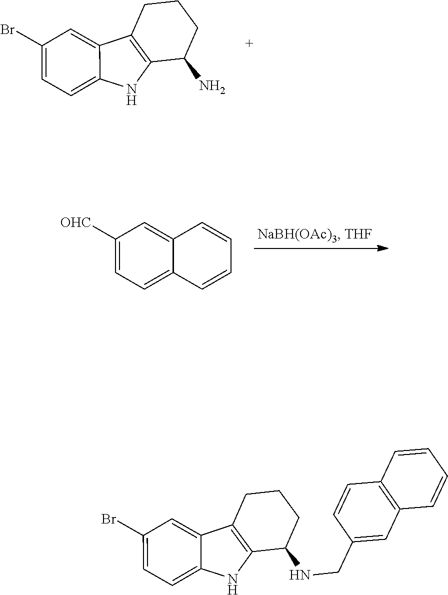 [SMILES: N[C@@H]1CCCc2c1[nH]c1ccc(Br)cc21 ; O=Cc1ccc2ccccc2c1 ; Brc1ccc2[nH]c3c(c2c1)CCC[C@H]3NCc1ccc2ccccc2c1]

To a solution of (R)-6-bromo-2,3,4,9-tetrahydro-1H-carbazol-1-amine (100 mg) and 2-naphthaldehyde (89 mg) in tetrahydrofuran 10 mL, was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 60 mg of (R)-6-bromo-N-(naphthalen-2-ylmethyl)-2,3,4,9-tetrahydro-1H-carbazol-1-amine.

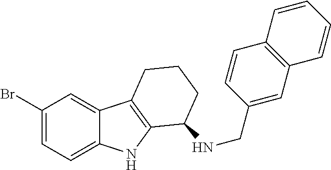 [SMILES: Brc1ccc2[nH]c3c(c2c1)CCC[C@H]3NCc1ccc2ccccc2c1]

### (R)-6-bromo-N-(naphthalen-2-ylmethyl)-2,3,4,9-tetrahydro-1H-carbazol-1-amine

¹H NMR (300 MHz, DMSO-d₆): δ 11.17 (s, 1H), 9.23 (m, 2H), 8.06 (s, 1H), 7.92 (m, 3H), 7.69 (s, 1H), 7.64 (d, J=6.0 Hz, 1H), 7.55 (d, J=6.0 Hz, 2H), 7.40 (d, J=6.0 Hz, 1H), 7.25 (d, J=6.0 Hz, 1H), 4.69 (broad s, 1H), 4.52 (m, 1H), 4.46 (m, 1H), 2.74 (m, 1H), 2.61 (m, 1H), 2.24 (m, 1H), 2.13 (m, 1H), 2.02 (m, 1H), 1.84 (m, 1H). Method [1] retention time 2.08 min, MS(ESI positive) 248 and 250 (M-NHBn), MS(ESI negative) 403 and 405 (M−H).

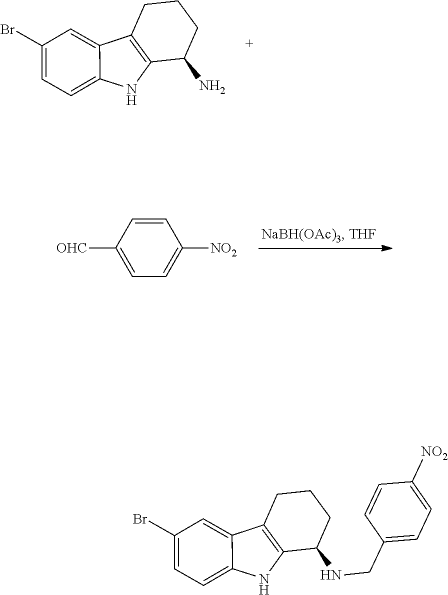 [SMILES: N[C@@H]1CCCc2c1[nH]c1ccc(Br)cc21 ; O=Cc1ccc([N+](=O)[O-])cc1 ; O=[N+]([O-])c1ccc(CN[C@@H]2CCCc3c2[nH]c2ccc(Br)cc32)cc1]

To a solution of (R)-6-bromo-2,3,4,9-tetrahydro-1H-carbazol-1-amine (100 mg) and 4-nitrobenzaldehyde (137 mg) in tetrahydrofuran (20 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 80 mg of (R)-6-bromo-N-(4-nitrobenzyl)-2,3,4,9-tetrahydro-1H-carbazol-1-amine.

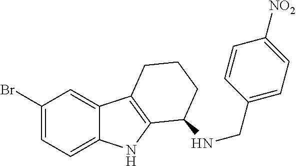 [SMILES: O=[N+]([O-])c1ccc(CN[C@@H]2CCCc3c2[nH]c2ccc(Br)cc32)cc1]

### (R)-6-bromo-N-(4-nitrobenzyl)-2,3,4,9-tetrahydro-1H-carbazol-1-amine

¹H NMR (300 MHz, DMSO-d₆): δ 11.10 (s, 1H), 9.23 (broad s, 2H), 8.29 (d, J=6.0 Hz, 2H), 7.78 (d, J=6.0 Hz, 2H), 7.68 (s, 1H), 7.38 (d, J=6.0 Hz, 1H), 7.25 (d, J=6.0 Hz, 1H), 4.66 (broad s, 1H), 4.52 (m, 1H), 4.44 (m, 1H), 2.72 (m, 1H), 2.61 (m, 1H), 2.21 (m, 1H), 2.10 (m, 1H), 1.99 (m, 1H), 1.84 (m, 1H). Method [1] retention time 1.96 min, MS(ESI positive) 248 and 250 (M-NHCH₂Ar), MS(ESI negative) 398 and 400 (M−H).

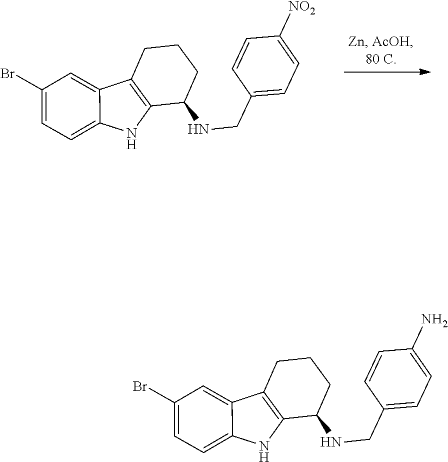 [SMILES: O=[N+]([O-])c1ccc(CN[C@@H]2CCCc3c2[nH]c2ccc(Br)cc32)cc1 ; Nc1ccc(CN[C@@H]2CCCc3c2[nH]c2ccc(Br)cc32)cc1]

To a solution of (R)-6-bromo-N-(4-nitrobenzyl)-2,3,4,9-tetrahydro-1H-carbazol-1-amine (150 mg, 0.376 mmol, 1.0 eq) in methanol (5 mL) and glacial acetic acid (0.5 mL), was added Zn (244 mg, 3.76 mmol) at room temperature. The reaction was monitored by liquid chromatography-mass spectrometry and thin layer chromatography. Filtered, concentrated, the residue was dissolved in dichloromethane, washed with saturated aqueous sodium bicarbonate and brine, dried over anhydrous sodium sulfate, concentrated, purified by prep-high pressure liquid chromatography to afford 87 mg of (R)—N-(4-aminobenzyl)-6-bromo-2,3,4,9-tetrahydro-1H-carbazol-1-amine.

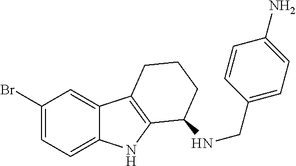 [SMILES: Nc1ccc(CN[C@@H]2CCCc3c2[nH]c2ccc(Br)cc32)cc1]

### (R)—N-(4-aminobenzyl)-6-bromo-2,3,4,9-tetrahydro-1H-carbazol-1-amine

¹H NMR (300 MHz, DMSO-d₆): δ 11.24 (d, J=3.0 Hz, 1H), 9.08 (broad s, 2H), 7.66 (s, 1H), 7.36 (d, J=6.0 Hz, 1H), 7.24 (m, 3H), 6.79 (m, 2H), 4.59 (broad s, 1H), 4.18 (m, 2H), 2.71 (m, 1H), 2.61 (m, 1H), 2.16-2.00 (m, 3H), 1.79 (m, 1H). Method [1] retention time 2.23 min, MS(ESI positive) 248 and 250 (M-NHCH₂Ar), MS(ESI negative) 368 and 370 (M−H).

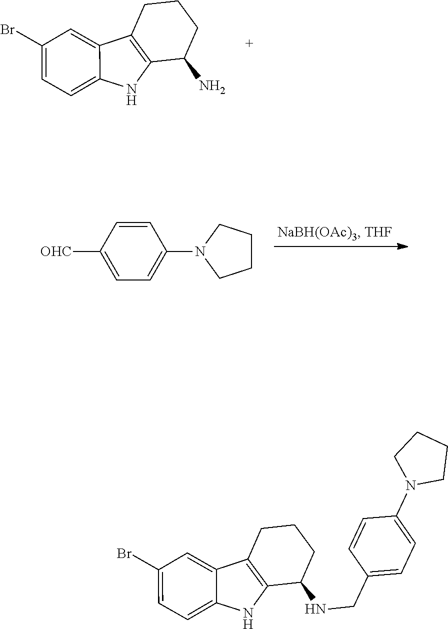 [SMILES: N[C@@H]1CCCc2c1[nH]c1ccc(Br)cc21 ; O=Cc1ccc(N2CCCC2)cc1 ; Brc1ccc2[nH]c3c(c2c1)CCC[C@H]3NCc1ccc(N2CCCC2)cc1]

To a solution of (R)-6-bromo-2,3,4,9-tetrahydro-1H-carbazol-1-amine (100 mg) and 4-(pyrrolidin-1-yl)benzaldehyde (80 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-thin layer chromatography.

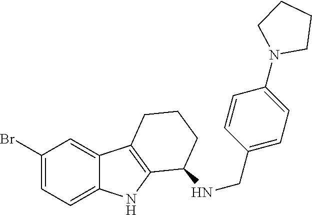 [SMILES: Brc1ccc2[nH]c3c(c2c1)CCC[C@H]3NCc1ccc(N2CCCC2)cc1]

### (R)-6-bromo-N-(4-(pyrrolidin-1-yl)benzyl)-2,3,4,9-tetrahydro-1H-carbazol-1-amine

¹H NMR (300 MHz, DMSO-d₆): δ 11.10 (d, J=4.2 Hz, 1H), 8.91 (broad s, 2H), 7.67 (s, 1H), 7.38 (d, J=6.3 Hz, 1H), 7.26 (m, 3H), 6.52 (d, J=6.6 Hz, 2H), 4.56 (broad s, 1H), 4.17 (m, 2H), 3.18 (broad s, 4H), 2.71 (m, 1H), 2.62 (m, 1H), 2.15-1.90 (m, 7H), 1.78 (m, 1H). Method [1] retention time 2.11 min, MS(ESI positive) 248 and 250 (M-NHCH₂Ar), MS(ESI negative) 423 and 425 (M−H).

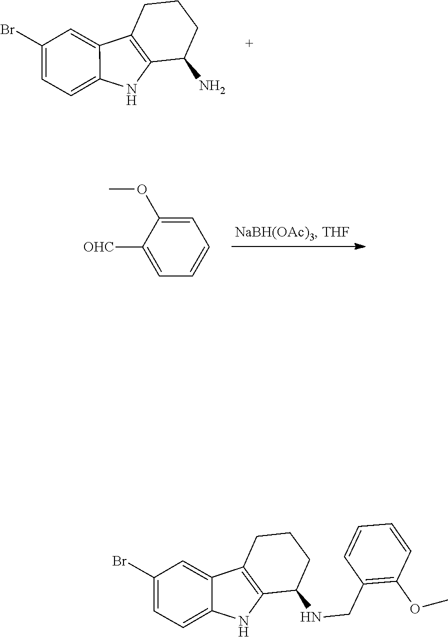

To a solution of (R)-6-bromo-2,3,4,9-tetrahydro-1H-carbazol-1-amine (100 mg) and 2-methoxybenzaldehyde (77 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 60 mg of (R)-6-bromo-N-(2-methoxybenzyl)-2,3,4,9-tetrahydro-1H-carbazol-1-amine.

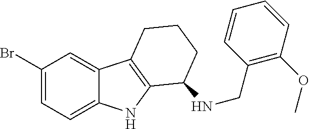 [SMILES: COc1ccccc1CN[C@@H]1CCCc2c1[nH]c1ccc(Br)cc21]

### (R)-6-bromo-N-(2-methoxybenzyl)-2,3,4,9-tetrahydro-1H-carbazol-1-amine

¹H NMR (300 MHz, DMSO-d₆): δ 11.10 (s, 1H), 9.02 (broad s, 1H), 8.90 (broad s, 1H), 7.67 (s, 1H), 7.38 (m, 3H), 7.24 (d, J=6.6 Hz, 1H), 7.06 (d, J=6.3 Hz, 1H), 6.52 (t, J=5.4 Hz, 1H), 4.66 (broad s, 1H), 4.23 (m, 2H), 3.78 (s, 3H), 2.72 (m, 1H), 2.62 (m, 1H), 2.17 (m, 2H), 2.04 (m, 1H), 1.81 (m, 1H). Method [1] retention time 2.03 min, MS(ESI positive) 385 and 387 (M+H).

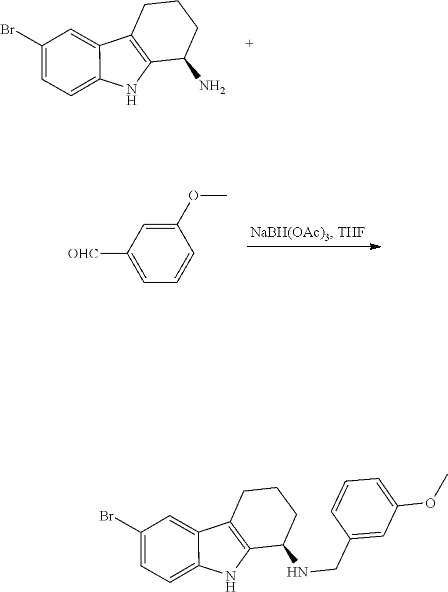

To a solution of (R)-6-bromo-2,3,4,9-tetrahydro-1H-carbazol-1-amine (100 mg) and 3-methoxybenzaldehyde (77 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 60 mg of (R)-6-bromo-N-(3-methoxybenzyl)-2,3,4,9-tetrahydro-1H-carbazol-1-amine.

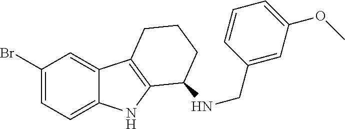 [SMILES: COc1cccc(CN[C@@H]2CCCc3c2[nH]c2ccc(Br)cc32)c1]

### (R)-6-bromo-N-(3-methoxybenzyl)-2,3,4,9-tetrahydro-1H-carbazol-1-amine

¹H NMR (300 MHz, DMSO-d₆): δ 11.10 (m, 1H), 9.11 (broad s, 2H), 7.68 (s, 1H), 7.28 (m, 3H), 7.24 (d, J=6.0 Hz, 1H), 7.12 (s, 1H), 7.07 (d, J=6.0 Hz, 1H), 6.96 (d, J=6.0 Hz, 1H), 4.62 (broad s, 1H), 4.32 (m, 1H), 4.26 (m, 1H), 3.74 (s, 3H), 2.71 (m, 1H), 2.62 (m, 1H), 2.18-2.00 (m, 3H), 1.82 (m, 1H). Method [1] retention time 2.00 min, MS(ESI positive) 248 and 250 (M-NHCH₂Ar), MS(ESI negative) 383 and 385 (M−H).

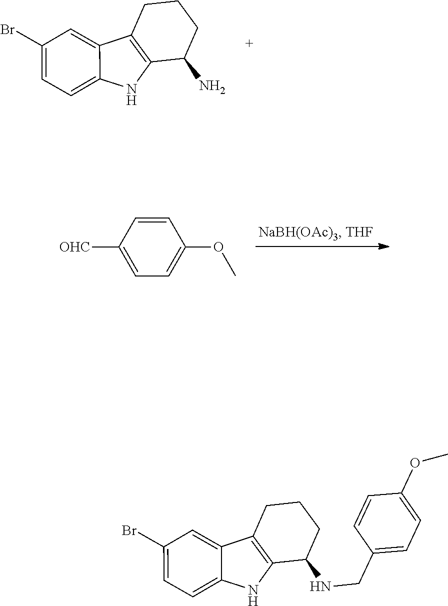

To a solution of (R)-6-bromo-2,3,4,9-tetrahydro-1H-carbazol-1-amine (100 mg) and 4-methoxybenzaldehyde (77 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to give 90 mg of (R)-6-bromo-N-(4-methoxybenzyl)-2,3,4,9-tetrahydro-1H-carbazol-1-amine.

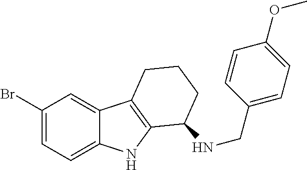 [SMILES: COc1ccc(CN[C@@H]2CCCc3c2[nH]c2ccc(Br)cc32)cc1]

### (R)-6-bromo-N-(4-methoxybenzyl)-2,3,4,9-tetrahydro-1H-carbazol-1-amine

¹H NMR (300 MHz, DMSO-d₆): δ 11.16 (s, 1H), 9.06 (broad s, 2H), 7.68 (s, 1H), 7.40 (d, J=6.0 Hz, 2H), 7.37 (d, J=6.0 Hz, 1H), 7.24 (d, J=6.0 Hz, 1H), 6.96 (d, J=6.0 Hz, 2H), 4.59 (broad s, 1H), 4.24 (m, 2H), 3.72 (s, 3H), 2.72 (m, 1H), 2.61 (m, 1H), 2.17-1.99 (m, 3H), 1.81 (m, 1H). Method [1] retention time 1.95 min, MS(ESI positive) 248 and 250 (M-NHCH₂Ar), MS(ESI negative) 383 and 385 (M−H).

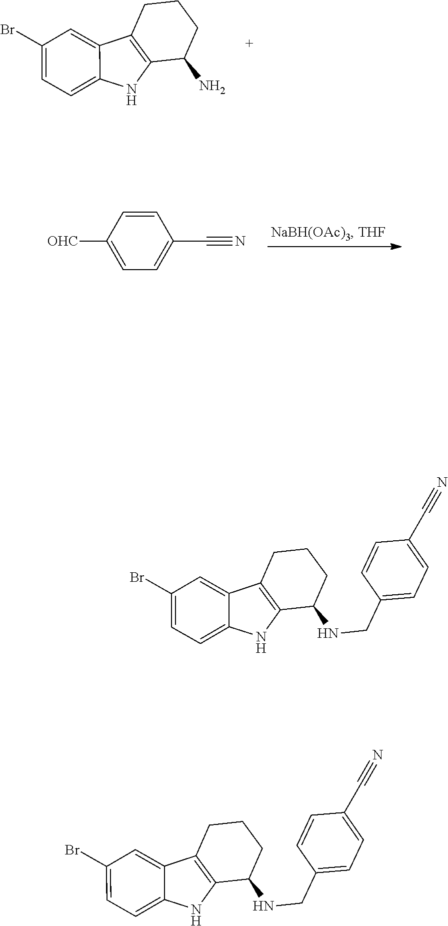 [SMILES: N[C@@H]1CCCc2c1[nH]c1ccc(Br)cc21 ; N#Cc1ccc(C=O)cc1 ; N#Cc1ccc(CN[C@@H]2CCCc3c2[nH]c2ccc(Br)cc32)cc1 ; N#Cc1ccc(CN[C@@H]2CCCc3c2[nH]c2ccc(Br)cc32)cc1]

### (R)-4-(((6-bromo-2,3,4,9-tetrahydro-1H-carbazol-1-yl)amino)methyl)benzonitrile

¹H NMR (300 MHz, DMSO-d₆): δ 11.17 (s, 1H), 9.27 (broad s, 2H), 7.91 (m, 2H), 7.70 (m, 3H), 7.38 (d, J=6.0 Hz, 1H), 7.24 (d, J=6.0 Hz, 1H), 4.64 (broad s, 1H), 4.45 (m, 1H), 4.37 (m, 1H), 2.73 (m, 1H), 2.60 (m, 1H), 2.19 (m, 1H), 2.10-1.99 (m, 2H), 1.81 (m, 1H). Method [1] retention time 1.93 min, MS(ESI positive) 248 and 250 (M-NHCH₂Ar), MS(ESI negative) 378 and 380 (M−H).

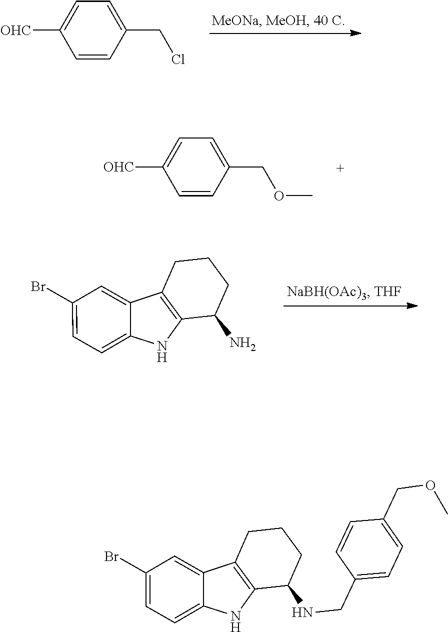 [SMILES: O=Cc1ccc(CCl)cc1 ; COCc1ccc(C=O)cc1 ; N[C@@H]1CCCc2c1[nH]c1ccc(Br)cc21 ; COCc1ccc(CN[C@@H]2CCCc3c2[nH]c2ccc(Br)cc32)cc1]

To a solution of 1 (100 mg) in methanol (5 mL), the solution of 5M sodium methoxide in methanol was added at room temperature. The reaction mixture was stirred for 16 h at room temperature. The reaction was monitored by thin layer chromatography. The reaction was poured into dichloromethane (30 mL) and water (10 mL). Separated the organic layer, dried over anhydrous sodium sulfate, concentrated, purified by prep-thin layer chromatography for the product 30 mg.

To a solution of (R)-6-bromo-2,3,4,9-tetrahydro-1H-carbazol-1-amine (100 mg) and 4-(methoxymethyl)benzaldehyde (85 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford (R)-6-bromo-N-(4-(methoxymethyl)benzyl)-2,3,4,9-tetrahydro-1H-carbazol-1-amine.

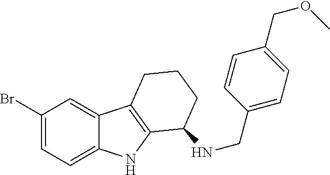 [SMILES: COCc1ccc(CN[C@@H]2CCCc3c2[nH]c2ccc(Br)cc32)cc1]

### (R)-6-bromo-N-(4-(methoxymethyl)benzyl)-2,3,4,9-tetrahydro-1H-carbazol-1-amine

¹H NMR (300 MHz, DMSO-d₆): δ 11.11 (s, 1H), 9.12 (broad s, 2H), 7.67 (s, 1H), 7.49 (d, J=6.0 Hz, 2H), 7.37 (m, 3H), 7.24 (d, J=6.0 Hz, 1H), 4.62 (broad s, 1H), 4.40 (s, 2H), 4.34 (m, 1H), 4.28 (m, 1H), 3.26 (s, 3H), 2.70 (m, 1H), 2.60 (m, 1H), 2.18-2.00 (m, 3H), 1.81 (m, 1H). Method [1] retention time 1.99 min, MS(ESI positive) 248 and 250 (M-NHCH₂Ar), MS(ESI negative) 397 and 399 (M−H).

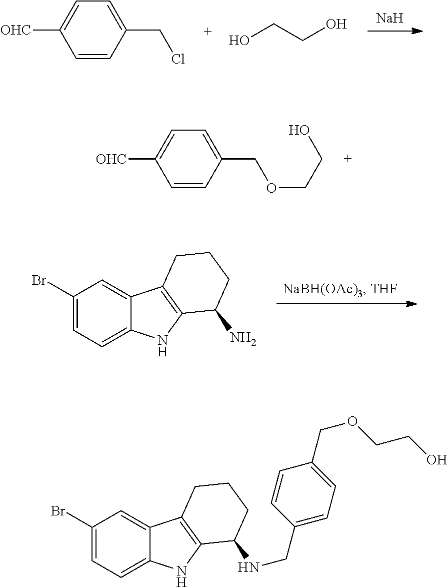

To a solvent ethylene glycol (10 mL), sodium hydride (258 mg) was added at room temperature. The mixture was stirred under nitrogen atmosphere for 1 h. Then, 1 (100 mg) was added at room temperature. The mixture was stirred for 16 h. The reaction was quenched with ice-water, extracted with ethyl acetate, washed with brine, dry over anhydrous sodium sulfate, concentrated, purified by prep-thin layer chromatography to afford 30 mg 4-((2-hydroxyethoxy)methyl)benzaldehyde.

To a solution of (R)-6-bromo-2,3,4,9-tetrahydro-1H-carbazol-1-amine (100 mg) and 4-((2-hydroxyethoxy)methyl)benzaldehyde (102 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to yield (R)-2-((4-(((6-bromo-2,3,4,9-tetrahydro-1H-carbazol-1-yl)amino)methyl)benzyl)oxy)ethan-1-ol.

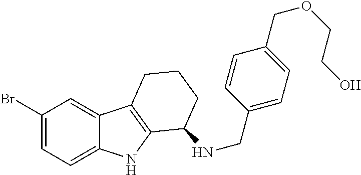 [SMILES: OCCOCc1ccc(CN[C@@H]2CCCc3c2[nH]c2ccc(Br)cc32)cc1]

(R)-2-((4-(((6-bromo-2,3,4,9-tetrahydro-1H-carbazol-1-yl)amino)methyl)benzyl)oxy)ethan-1-ol. ¹H NMR (300 MHz, DMSO-d₆): δ 11.11 (d, J=Hz, 1H), 9.09 (broad s, 2H), 7.68 (s, 1H), 7.49 (d, J=6.0 Hz, 2H), 7.28 (m, 3H), 7.24 (d, J=6.0 Hz, 1H), 4.62 (broad s, 1H), 4.40 (s, 2H), 4.34 (m, 1H), 4.27 (m, 1H), 3.51 (t, J=Hz, 2H), 3.42 (t, J=Hz, 2H), 2.60 (m, 1H), 2.58 (m, 1H), 2.18-2.00 (m, 3H), 1.82 (m, 1H). Method [1] retention time 1.88 min, MS(ESI positive) 451 and 453 (M+Na), MS(ESI negative) 427 and 429 (M−H).

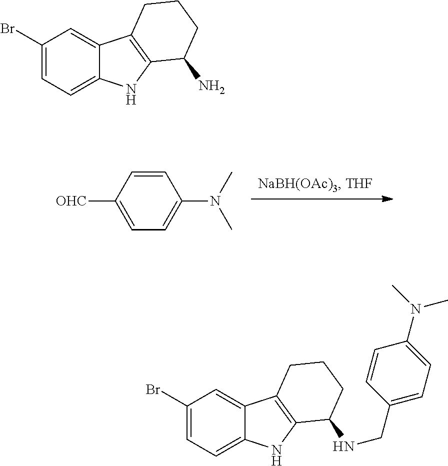 [SMILES: N[C@@H]1CCCc2c1[nH]c1ccc(Br)cc21 ; CN(C)c1ccc(C=O)cc1 ; CN(C)c1ccc(CN[C@@H]2CCCc3c2[nH]c2ccc(Br)cc32)cc1]

To a solution of (R)-6-bromo-2,3,4,9-tetrahydro-1H-carbazol-1-amine (100 mg) and 4-(dimethylamino)benzaldehyde (85 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to yield (R)-6-bromo-N-(4-(dimethylamino)benzyl)-2,3,4,9-tetrahydro-1H-carbazol-1-amine.

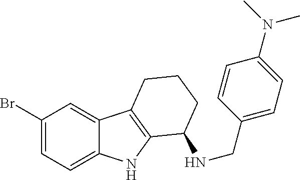 [SMILES: CN(C)c1ccc(CN[C@@H]2CCCc3c2[nH]c2ccc(Br)cc32)cc1]

### (R)-6-bromo-N-(4-(dimethylamino)benzyl)-2,3,4,9-tetrahydro-1H-carbazol-1-amine

¹H NMR (300 MHz, DMSO-d₆): δ 11.07 (s, 1H), 8.89 (broad s, 2H), 7.67 (s, 1H), 7.37 (d, J=6.0 Hz, 1H), 7.29 (d, J=6.0 Hz, 2H), 7.24 (d, J=6.0 Hz, 1H), 6.72 (d, J=6.0 Hz, 2H), 4.57 (broad s, 1H), 4.20 (m, 1H), 4.14 (m, 1H), 2.88 (s, 6H), 2.69 (m, 1H), 2.60 (m, 1H), 2.15 (m, 1H), 2.08 (m, 1H), 1.98 (m, 1H), 1.80 (m, 1H). Method [1] retention time 2.43 min.

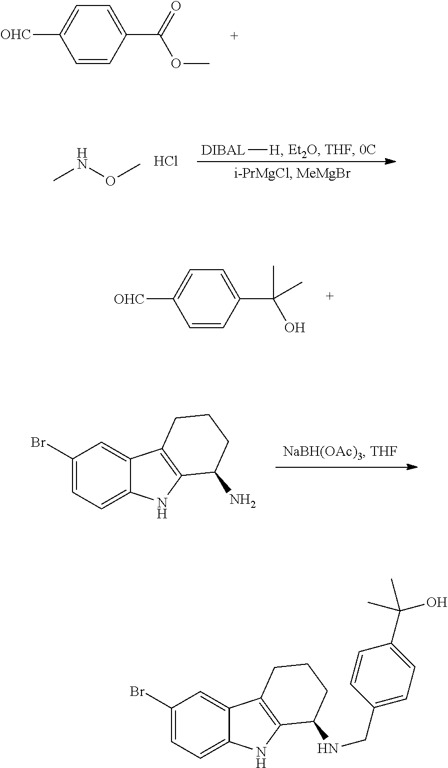

To a suspension solution of N,O-dimethylhydroxylamine hydrochloride (713 mg, 7.31 mmol) in 20 mL (diethyl ether/tetrahydrofuran=1:1) under nitrogen atmosphere at 0° C., was added a solution 1 M diisobutylaluminium hydride in hexanes (7.31 mL). The mixture solution was stirred for 30 min allowing the temperature slowly to warm to room temperature. Then, cooled to 0° C., was added methyl 4-formylbenzoate (1.0 g, 6.09 mmol, 1.0 eq in diethyl ether, 20 mL) and stirred for 2 h at room temperature. Then, a solution of 2M isopropyl magnesium chloride in tetrahydrofuran (3.7 mL) was added and stirred for 1 h at room temperature. Then, a solution of 3M methyl magnesium bromide in tetrahydrofuran (13.7 mL, 41.1 mmol) was added to above mixture solution and stirred for 4 h at room temperature. The solution was monitored by liquid chromatography-mass spectrometry and thin layer chromatography. Quenched by saturated aqueous ammonium chloride. Extracted with ethyl acetate and washed with brine, dry over anhydrous sodium sulfate, concentrated and purified by flash chromatography to yield 500 mg of 4-(2-hydroxypropan-2-yl)benzaldehyde as a colorless oil.

To a solution of (R)-6-bromo-2,3,4,9-tetrahydro-1H-carbazol-1-amine (100 mg) and 4-(2-hydroxypropan-2-yl)benzaldehyde (68 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-thin layer chromatography to afford 16 mg of (R)-2-(4-(((6-bromo-2,3,4,9-tetrahydro-1H-carbazol-1-yl)amino)methyl)phenyl)propan-2-ol as white solid.

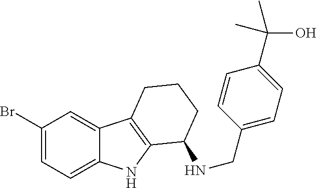 [SMILES: CC(C)(O)c1ccc(CN[C@@H]2CCCc3c2[nH]c2ccc(Br)cc32)cc1]

### (R)-2-(4-(((6-bromo-2,3,4,9-tetrahydro-1H-carbazol-1-yl)amino)methyl)phenyl)propan-2-ol

¹H NMR (300 MHz, DMSO-d₆): δ 11.60 (broad s, 1H), 9.67 (broad s, 2H), 7.65 (s, 1H), 7.49 (m, 4H), 7.25 (d, J=6.0 Hz, 1H), 7.22 (d, J=6.0 Hz, 1H), 5.04 (broad s, 1H), 4.64 (broad s, 1H), 4.21 (broad s, 1H), 4.28 (m, 1H), 2.64 (m, 2H), 2.15 (broad s, 2H), 2.05 (m, 1H), 1.77 (m, 1H), 1.38 (s, 6H). Method [1] retention time 1.98 min, MS(ESI positive) 248 and 250 (M-NHCH₂Ar), MS(ESI negative) 411 and 413 (M−H).

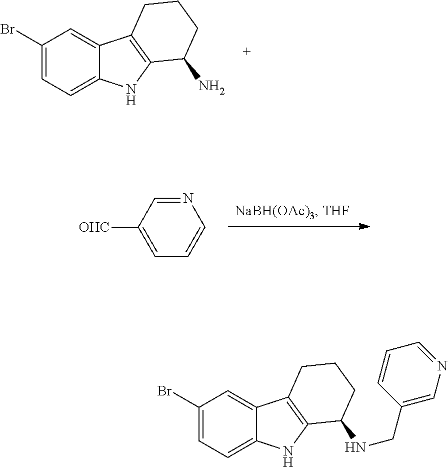 [SMILES: N[C@@H]1CCCc2c1[nH]c1ccc(Br)cc21 ; O=Cc1cccnc1 ; Brc1ccc2[nH]c3c(c2c1)CCC[C@H]3NCc1cccnc1]

To a solution of (R)-6-bromo-2,3,4,9-tetrahydro-1H-carbazol-1-amine (100 mg) and nicotinaldehyde (60.8 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford product 70 mg of (R)-6-bromo-N-(pyridin-3-ylmethyl)-2,3,4,9-tetrahydro-1H-carbazol-1-amine.

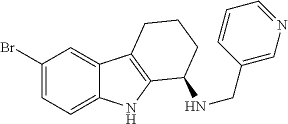 [SMILES: Brc1ccc2[nH]c3c(c2c1)CCC[C@H]3NCc1cccnc1]

### (R)-6-bromo-N-(pyridin-3-ylmethyl)-2,3,4,9-tetrahydro-1H-carbazol-1-amine

¹H NMR (300 MHz, DMSO-d₆): δ 11.10 (broad s, 1H), 9.10 (broad s, 2H), 8.70 (s, 1H), 8.60 (d, J=3.6 Hz, 1H), 7.95 (d, J=6.0 Hz, 1H), 7.68 (d, J=1.5 Hz, 1H), 7.48 (m, 1H), 7.39 (d, J=6.3 Hz, 1H), 7.25 (dd, J=6.3 Hz and 1.5 Hz, 1H), 4.65 (broad s, 1H), 4.47 (broad s, 1H), 4.43 (m, 1H), 2.74 (m, 1H), 2.59 (m, 1H), 2.13 (m, 1H), 2.07 (m, 1H), 1.98 (m, 1H), 1.80 (m, 1H). Method [1] retention time 1.71 min, MS(ESI positive) 356 and 358 (M+1), MS(ESI negative) 354 and 356 (M−H).

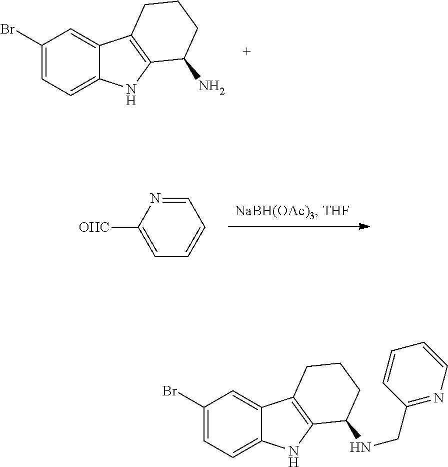 [SMILES: N[C@@H]1CCCc2c1[nH]c1ccc(Br)cc21 ; O=Cc1ccccn1 ; Brc1ccc2[nH]c3c(c2c1)CCC[C@H]3NCc1ccccn1]

To a solution of (R)-6-bromo-2,3,4,9-tetrahydro-1H-carbazol-1-amine (100 mg) and picolinaldehyde (60.8 mg) in tetrahydrofuran (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 105 mg of (R)-6-bromo-N-(pyridin-2-ylmethyl)-2,3,4,9-tetrahydro-1H-carbazol-1-amine.

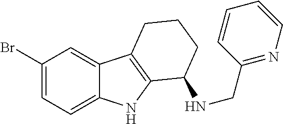 [SMILES: Brc1ccc2[nH]c3c(c2c1)CCC[C@H]3NCc1ccccn1]

### (R)-6-bromo-N-(pyridin-2-ylmethyl)-2,3,4,9-tetrahydro-1H-carbazol-1-amine

¹H NMR (300 MHz, DMSO-d₆): δ 11.02 (broad s, 1H), 9.37 (broad s, 2H), 8.65 (d, J=3.0 Hz, 1H), 7.88 (t, J=6.0 Hz, 1H), 7.67 (s, 1H), 7.51 (d, J=6.0 Hz, 1H), 7.43 (m, 2H), 7.24 (d, J=3.0 Hz, 1H), 4.68 (broad s, 1H), 4.47 (broad s, 2H), 2.64 (m, 1H), 2.48 (m, 1H), 2.20 (m, 1H), 2.12 (m, 1H), 2.02 (m, 1H), 1.82 (m, 1H). Method [1] retention time 2.00 min, MS(ESI positive) 248 and 250 (M-NHCH₂Ar), MS(ESI negative) 354 and 356 (M−H).

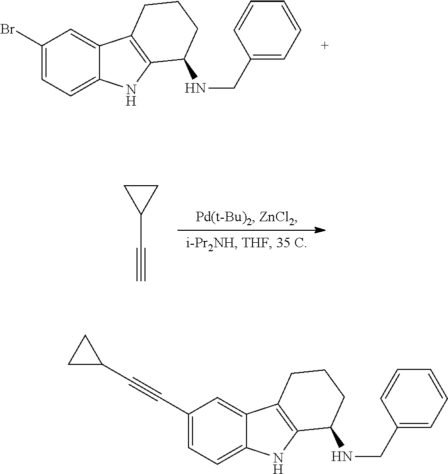

(R)—N-benzyl-6-bromo-2,3,4,9-tetrahydro-1H-carbazol-1-amine (100 mg), zinc chloride (30 mg) and bis(tri-tert-butylphosphine)palladium(0) (30 mg) was added into 50 mL Three-necked round bottom. Then, Nitrogen displacement three times, isopropylamine (3 mL) and tetrahydrofuran (7 mL) was added at 35 C. After 3 min, ethynylcyclopropane (0.2 mL) was added at 35 C. The reaction mixture was stirred for 16 h. The reaction was monitored by liquid chromatography-mass spectrometry and thin layer chromatography. Cooled to room temperature, the reaction mixture was filtered with celite. The filtration was concentrated and washed with saturated aqueous sodium bicarbonate, extracted with dichloromethane. The organic layer was dried by anhydrous sodium sulfate, concentrated, purified by prep-thin layer chromatography and prep-high pressure liquid chromatography to afford 8 mg of (R)—N-benzyl-6-(cyclopropylethynyl)-2,3,4,9-tetrahydro-1H-carbazol-1-amine.

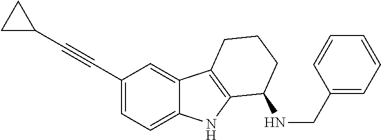 [SMILES: C(#CC1CC1)c1ccc2[nH]c3c(c2c1)CCC[C@H]3NCc1ccccc1]

### (R)—N-benzyl-6-(cyclopropylethynyl)-2,3,4,9-tetrahydro-1H-carbazol-1-amine

¹H NMR (300 MHz, DMSO-d₆): δ 11.0 (s, 1H), 9.06 (broad s, 2H), 7.50 (m, 3H), 7.40 (m, 3H), 7.35 (d, J=6.3 Hz, 1H), 7.10 (dd, J=6.3 and 1.2 Hz, 1H), 4.61 (broad s, 1H), 4.36 (m, 1H), 4.28 (m, 1H), 2.71 (m, 1H), 2.62 (m, 1H), 2.18 (m, 1H), 2.11 (m, 1H), 2.03 (m, 1H), 1.83 (m, 1H), 1.47 (m, 1H), 0.83 (m, 2H), 0.67 (m, 2H). Method [1] retention time 1.97 min, MS(ESI positive) 234 (M-NHBn), MS(ESI negative) and 339 (M−H).

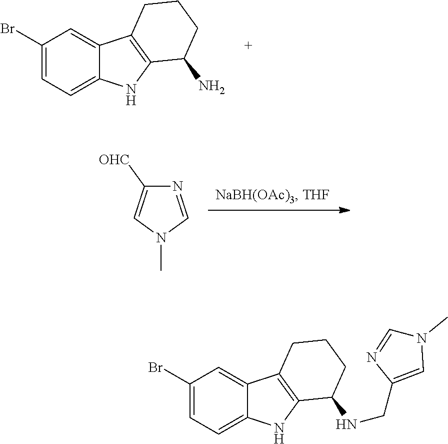

To a solution of (R)-6-bromo-2,3,4,9-tetrahydro-1H-carbazol-1-amine (150 mg) and 1-methyl-1H-imidazole-4-carbaldehyde (198 mg) in tetrahydrofuran (20 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, washed with brine, dry over anhydrous sodium sulfate, concentrated, purified by prep-high pressure liquid chromatography to afford 60 mg (R)-6-bromo-N-((1-methyl-1H-imidazol-5-yl)methyl)-2,3,4,9-tetrahydro-1H-carbazol-1-amine.

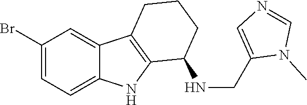 [SMILES: Cn1cncc1CN[C@@H]1CCCc2c1[nH]c1ccc(Br)cc21]

### (R)-6-bromo-N-((1-methyl-1H-imidazol-5-yl)methyl)-2,3,4,9-tetrahydro-1H-carbazol-1-amine

¹H NMR (300 MHz, DMSO-d₆): δ 11.16 (broad s, 1H), 8.84 (broad s, 1H), 7.66 (s, 1H), 7.61 (s, 1H), 7.35 (d, J=6.0 Hz, 1H), 7.23 (d, J=6.0 Hz, 1H), 4.58 (broad s, 1H), 4.38 (broad s, 2H), 3.82 (s, 3H), 2.70 (m, 1H), 2.62 (m, 1H), 2.11 (broad s, 2H), 1.97 (m, 1H), 1.84 (m, 1H). Method [1] retention time 1.48 min, MS(ESI positive) 359 and 361 (M+H), MS(ESI negative) 357 and 359 (M−H).

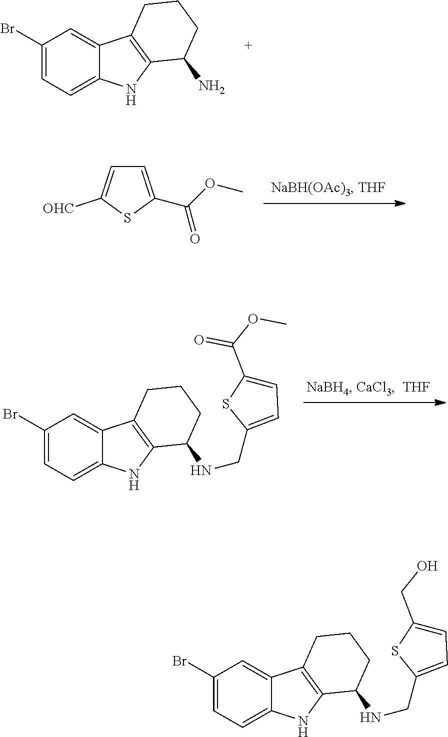 [SMILES: N[C@@H]1CCCc2c1[nH]c1ccc(Br)cc21 ; COC(=O)c1ccc(C=O)s1 ; COC(=O)c1ccc(CN[C@@H]2CCCc3c2[nH]c2ccc(Br)cc32)s1 ; OCc1ccc(CN[C@@H]2CCCc3c2[nH]c2ccc(Br)cc32)s1]

To a solution of (R)-6-bromo-2,3,4,9-tetrahydro-1H-carbazol-1-amine (150 mg) and methyl 5-formylthiophene-2-carboxylate (198 mg) in tetrahydrofuran (20 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, washed with brine, dry over anhydrous sodium sulfate, concentrated to yield 470 mg of methyl (R)-5-(((6-bromo-2,3,4,9-tetrahydro-1H-carbazol-1-yl)amino)methyl)thiophene-2-carboxylate.

To a solution of methyl (R)-5-(((6-bromo-2,3,4,9-tetrahydro-1H-carbazol-1-yl)amino)methyl)thiophene-2-carboxylate (200 mg) in tetrahydrofuran (20 mL), sodium borohydride (72 mg) was added at room temperature. Then calcium chloride (275 mg) was added at room temperature. The mixture was stirred for 16 h. The reaction was monitored by liquid chromatography-mass spectrometry and thin layer chromatography. Quenched with saturated aqueous ammonium chloride, extracted with ethyl acetate, washed with brine, dry over anhydrous sodium sulfate, concentrated and purified by prep-thin layer chromatography and prep-high pressure liquid chromatography to afford 68 mg of (R)-(5-(((6-bromo-2,3,4,9-tetrahydro-1H-carbazol-1-yl)amino)methyl)thiophen-2-yl)methanol.

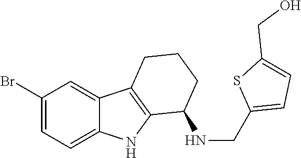 [SMILES: OCc1ccc(CN[C@@H]2CCCc3c2[nH]c2ccc(Br)cc32)s1]

(R)-(5-(((6-bromo-2,3,4,9-tetrahydro-1H-carbazol-1-yl)amino)methyl)thiophen-2-yl)methanol. ¹H NMR (300 MHz, DMSO-d₆): δ 11.05 (broad s, 1H), 9.19 (broad s, 2H), 7.67 (s, 1H), 7.35 (d, J=6.0 Hz, 1H), 7.26 (d, J=6.0 Hz, 1H), 7.13 (s, 1H), 6.89 (s, 1H), 4.59 (s, 2H), 4.53 (m, 1H), 4.47 (m, 1H), 2.70 (m, 1H), 2.58 (m, 1H), 2.18 (m, 1H), 2.05 (m, 1H), 1.97 (m, 1H), 1.81 (m, 1H). Method [1] retention time 1.85 min, MS(ESI positive) 248 and 250 (M-NHCH₂Ar), MS(ESI negative) 389 and 391 (M−H).

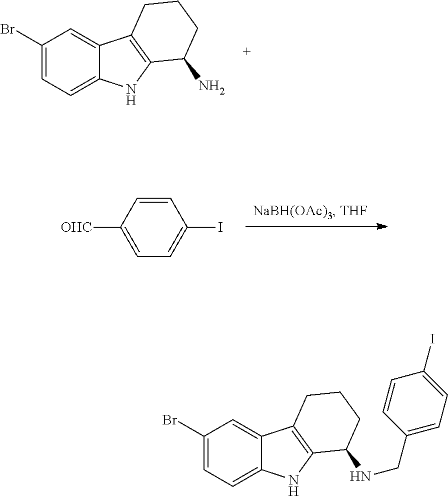 [SMILES: N[C@@H]1CCCc2c1[nH]c1ccc(Br)cc21 ; O=Cc1ccc(I)cc1 ; Brc1ccc2[nH]c3c(c2c1)CCC[C@H]3NCc1ccc(I)cc1]

To a solution of (R)-6-bromo-2,3,4,9-tetrahydro-1H-carbazol-1-amine (150 mg) and 4-iodobenzaldehyde (131 mg) in tetrahydrofuran (20 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 120 mg of (R)-6-bromo-N-(4-iodobenzyl)-2,3,4,9-tetrahydro-1H-carbazol-1-amine.

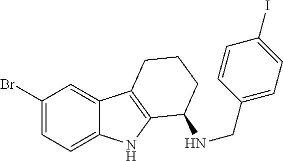 [SMILES: Brc1ccc2[nH]c3c(c2c1)CCC[C@H]3NCc1ccc(I)cc1]

(R)-6-bromo-N-(4-iodobenzyl)-2,3,4,9-tetrahydro-1H-carbazol-1-amine. ¹H NMR (300 MHz, DMSO-d₆): δ 11.14 (broad s, 1H), 9.16 (broad s, 2H), 7.79 (d, J=6.0 Hz, 2H), 7.67 (s, 1H), 7.37 (d, J=6.0 Hz, 1H), 7.39 (d, J=6.0 Hz, 1H), 7.31 (d, J=6.0 Hz, 2H), 4.61 (broad s, 1H), 4.29 (m, 1H), 4.24 (m, 1H), 2.70 (m, 1H), 2.61 (m, 1H), 2.16 (m, 1H), 2.04 (m, 1H), 1.99 (m, 1H), 1.81 (m, 1H). Method [1] retention time 2.00 min, MS(ESI positive) 248 and 250 (M-NHCH₂Ar), MS(ESI negative) 479 and 481 (M−H).

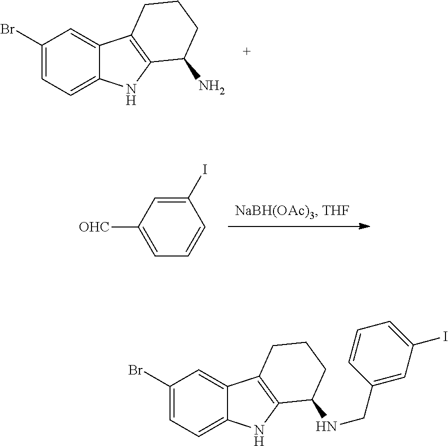 [SMILES: N[C@@H]1CCCc2c1[nH]c1ccc(Br)cc21 ; O=Cc1cccc(I)c1 ; Brc1ccc2[nH]c3c(c2c1)CCC[C@H]3NCc1cccc(I)c1]

To a solution of (R)-6-bromo-2,3,4,9-tetrahydro-1H-carbazol-1-amine (150 mg) and 3-iodobenzaldehyde (131 mg) in tetrahydrofuran (20 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 140 mg of (R)-6-bromo-N-(3-iodobenzyl)-2,3,4,9-tetrahydro-1H-carbazol-1-amine.

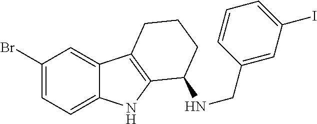 [SMILES: Brc1ccc2[nH]c3c(c2c1)CCC[C@H]3NCc1cccc(I)c1]

(R)-6-bromo-N-(3-iodobenzyl)-2,3,4,9-tetrahydro-1H-carbazol-1-amine. ¹H NMR (300 MHz, DMSO-d₆): δ 11.13 (broad s, 1H), 9.13 (broad s, 2H), 7.93 (s, 1H), 7.75 (d, J=6.0 Hz, 1H), 7.67 (s, 1H), 7.51 (d, J=6.0 Hz, 1H), 7.38 (d, J=6.0 Hz, 1H), 7.21 (m, 2H), 4.62 (broad s, 1H), 4.32 (m, 1H), 4.24 (m, 1H), 2.71 (m, 1H), 2.58 (m, 1H), 2.19 (m, 1H), 2.06 (m, 1H), 1.99 (m, 1H), 1.82 (m, 1H). Method [1] retention time 2.02 min, MS(ESI positive) 248 and 250 (M-NHCH₂Ar), MS(ESI negative) 479 and 481 (M−H).

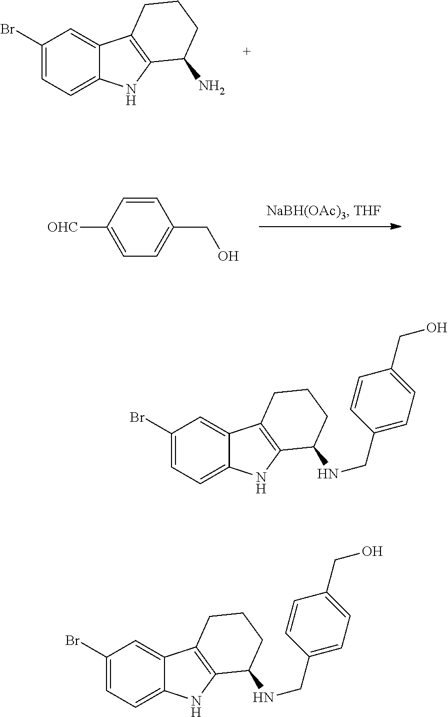 [SMILES: N[C@@H]1CCCc2c1[nH]c1ccc(Br)cc21 ; O=Cc1ccc(CO)cc1 ; OCc1ccc(CN[C@@H]2CCCc3c2[nH]c2ccc(Br)cc32)cc1 ; OCc1ccc(CN[C@@H]2CCCc3c2[nH]c2ccc(Br)cc32)cc1]

(R)-(4-(((6-bromo-2,3,4,9-tetrahydro-1H-carbazol-1-yl)amino)methyl)phenyl)methanol. ¹H NMR (300 MHz, DMSO-d₆): δ 11.66 (s, 1H), 9.91 (broad s, 1H), 9.75 (broad s, 1H), 7.65 (s, 1H), 7.58 (d, J=5.7 Hz, 2H), 7.34 (m, 3H), 7.21 (d, J=6.3 Hz, 1H), 5.73 (broad s, 1H), 4.63 (broad s, 1H), 4.48 (m, 2H), 4.22 (m, 2H), 2.63 (m, 2H), 2.18 (m, 2H), 2.05 (m, 1H), 1.75 (m, 1H). Method [1] retention time 1.83 min, MS(ESI positive) 248 and 250 (M-NHCH₂Ar), MS(ESI negative) 383 and 385 (M−H).

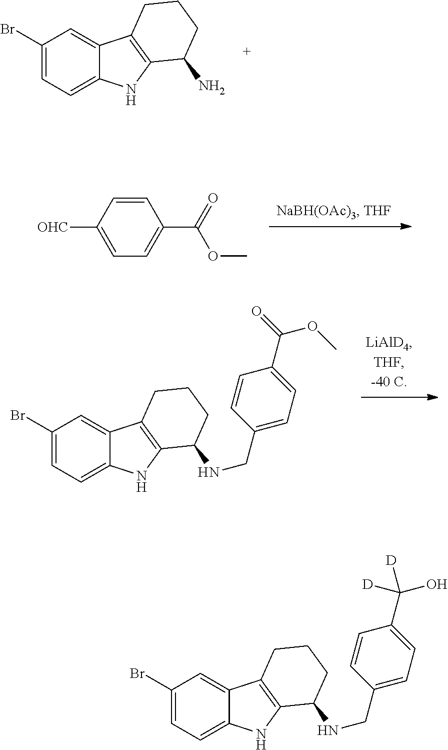 [SMILES: N[C@@H]1CCCc2c1[nH]c1ccc(Br)cc21 ; COC(=O)c1ccc(C=O)cc1 ; COC(=O)c1ccc(CN[C@@H]2CCCc3c2[nH]c2ccc(Br)cc32)cc1 ; [2H]C([2H])(O)c1ccc(CN[C@@H]2CCCc3c2[nH]c2ccc(Br)cc32)cc1]

To a solution of (R)-6-bromo-2,3,4,9-tetrahydro-1H-carbazol-1-amine (100 mg) and methyl 4-formylbenzoate (93 mg) in methanol (10 mL), was added sodium triacetoxyborohydride at room temperature. The mixture was stirred for 16 h. The reaction was monitored by liquid chromatography-mass spectrometry and thin layer chromatography. Quenched with saturated aqueous ammonium chloride, extracted with ethyl acetate, washed with brine, dry over anhydrous sodium sulfate, concentrated to yield 470 mg of methyl (R)-4-(((6-bromo-2,3,4,9-tetrahydro-1H-carbazol-1-yl)amino)methyl)benzoate.

To a solution of methyl (R)-4-(((6-bromo-2,3,4,9-tetrahydro-1H-carbazol-1-yl)amino)methyl)benzoate (80 mg) in tetrahydrofuran (20 mL), lithium aluminium deuteride (28 mg) was added at −40 C. The reaction was stirred for 3 h at room temperature. The reaction was monitored by liquid chromatography-mass spectrometry and thin layer chromatography.

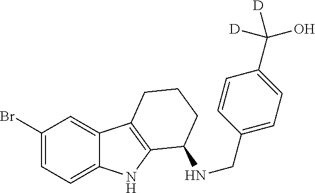 [SMILES: [2H]C([2H])(O)c1ccc(CN[C@@H]2CCCc3c2[nH]c2ccc(Br)cc32)cc1]

### (R)-(4-(((6-bromo-2,3,4,9-tetrahydro-1H-carbazol-1-yl)amino)methyl)phenyl)methan-d₂-ol

¹H NMR (300 MHz, DMSO-d₆): δ 11.08 (broad s, 1H), 9.08 (broad s, 2H), 7.67 (s, 1H), 7.47 (d, J=6.0 Hz, 2H), 7.38 (m, 3H), 7.25 (d, J=6.0 Hz, 1H), 4.62 (broad s, 1H), 4.32 (m, 1H), 4.28 (m, 1H), 2.71 (m, 1H), 2.63 (m, 1H), 2.19 (m, 1H), 2.05 (m, 1H), 2.01 (m, 1H), 1.82 (m, 1H). Method [1] retention time 1.86 min, MS(ESI positive) 248 and 250 (M-NHCH₂Ar), MS(ESI negative) 385 and 387 (M−H).

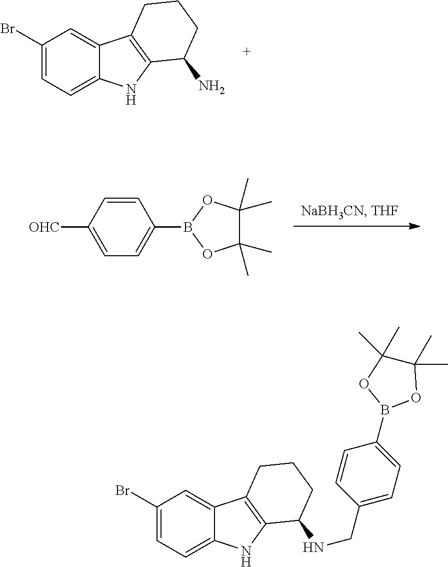 [SMILES: N[C@@H]1CCCc2c1[nH]c1ccc(Br)cc21 ; CC1(C)OB(c2ccc(C=O)cc2)OC1(C)C ; CC1(C)OB(c2ccc(CN[C@@H]3CCCc4c3[nH]c3ccc(Br)cc43)cc2)OC1(C)C]

To a solution of (R)-6-bromo-2,3,4,9-tetrahydro-1H-carbazol-1-amine (150 mg) and 4-(4,4,5,5-tetramethyl-1,3,2-dioxaborolan-2-yl)benzaldehyde (198 mg) in methanol (10 mL), was added sodium cyanoborohydride and 3 drops glacial acetic acid at room temperature under nitrogen atmosphere, the mixture was stirred for 16 h. The reaction was monitored by liquid chromatography-mass spectrometry and thin layer chromatography. Poured into saturated aqueous sodium bicarbonate, extracted with ethyl acetate, dry over anhydrous sodium sulfate, concentrated, purified by prep-high pressure liquid chromatography to afford 80 mg of (R)-6-bromo-N-(4-(4,4,5,5-tetramethyl-1,3,2-dioxaborolan-2-yl)benzyl)-2,3,4,9-tetrahydro-1H-carbazol-1-amine.

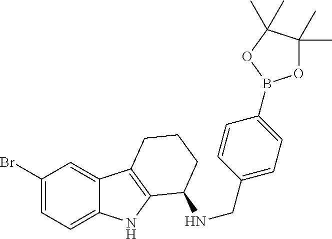 [SMILES: CC1(C)OB(c2ccc(CN[C@@H]3CCCc4c3[nH]c3ccc(Br)cc43)cc2)OC1(C)C]

(R)-6-bromo-N-(4-(4,4,5,5-tetramethyl-1,3,2-dioxaborolan-2-yl)benzyl)-2,3,4,9-tetrahydro-1H-carbazol-1-amine. ¹H NMR (300 MHz, DMSO-d₆): δ10.86 (broad s, 1H), 7.60 (d, J=6.0 Hz, 2H), 7.48 (s, 1H), 7.44 (d, J=6.0 Hz, 2H), 7.24 (d, J=6.0 Hz, 1H), 7.09 (d, J=6.0 Hz, 1H), 3.83 (m, 3H), 2.54 (broad s, 2H), 1.97 (m, 2H), 1.66 (m, 2H) 1.26 (s, 12H). Method [1] retention time 2.16 min, MS(ESI positive) 248 and 250 (M-NHCH₂Ar), MS(ESI negative) 479 and 481 (M−H).

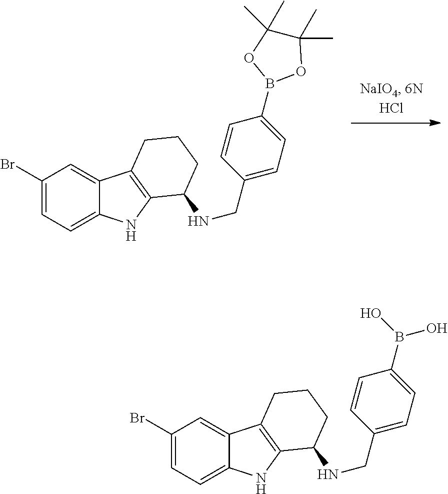 [SMILES: CC1(C)OB(c2ccc(CN[C@@H]3CCCc4c3[nH]c3ccc(Br)cc43)cc2)OC1(C)C ; OB(O)c1ccc(CN[C@@H]2CCCc3c2[nH]c2ccc(Br)cc32)cc1]

To a solution of (R)-6-bromo-N-(4-(4,4,5,5-tetramethyl-1,3,2-dioxaborolan-2-yl)benzyl)-2,3,4,9-tetrahydro-1H-carbazol-1-amine (80 mg) in tetrahydrofuran (10 mL), was 6 N hydrogen chloride (10 mL) added at room temperature. The reaction was stirred for 16 h. The reaction was monitored by liquid chromatography-mass spectrometry. Concentrated for the product, purified by prep-high pressure liquid chromatography to afford 30 mg of (R)-(4-(((6-bromo-2,3,4,9-tetrahydro-1H-carbazol-1-yl)amino)methyl)phenyl)boronic acid as a white solid.

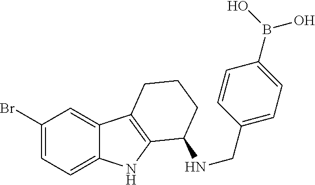 [SMILES: OB(O)c1ccc(CN[C@@H]2CCCc3c2[nH]c2ccc(Br)cc32)cc1]

(R)-(4-(((6-bromo-2,3,4,9-tetrahydro-1H-carbazol-1-yl)amino)methyl)phenyl)boronic acid. ¹H NMR (300 MHz, DMSO-d₆): δ 11.04 (broad s, 1H), 9.08 (broad s, 2H), 8.13 (broad s, 2H), 7.68 (d, J=6.0 Hz, 2H), 7.48 (s, 1H), 7.46 (d, J=6.0 Hz, 2H), 7.39 (d, J=6.0 Hz, 1H), 7.25 (d, J=6.0 Hz, 1H), 4.63 (broad s, 1H), 4.35 (m, 1H), 4.29 (m, 1H), 2.71 (m, 1H), 2.58 (m, 1H), 2.19 (m, 1H), 2.10 (m, 1H), 2.00 (m, 1H), 1.82 (m, 1H). Method [1] retention time 1.84 min, MS(ESI positive) 248 and 250 (M-NHCH₂Ar), MS(ESI negative) 397 and 399 (M−H).

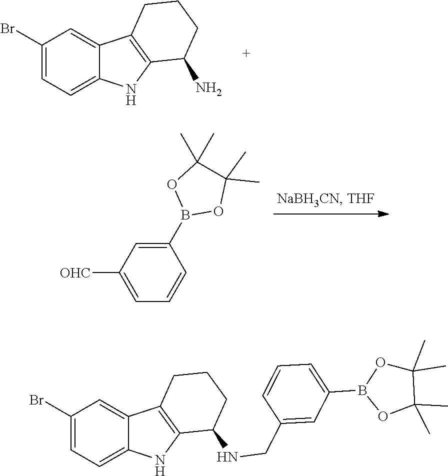 [SMILES: N[C@@H]1CCCc2c1[nH]c1ccc(Br)cc21 ; CC1(C)OB(c2cccc(C=O)c2)OC1(C)C ; CC1(C)OB(c2cccc(CN[C@@H]3CCCc4c3[nH]c3ccc(Br)cc43)c2)OC1(C)C]

To a solution of (R)-6-bromo-2,3,4,9-tetrahydro-1H-carbazol-1-amine (150 mg) and 3-(4,4,5,5-tetramethyl-1,3,2-dioxaborolan-2-yl)benzaldehyde (198 mg) in methanol 10 mL, was added sodium cyanoborohydride and 3 drops glacial acetic acid at room temperature under nitrogen atmosphere, the mixture was stirred for 16 h. The reaction was monitored by liquid chromatography-mass spectrometry and thin layer chromatography. Poured into saturated aqueous sodium bicarbonate, extracted with ethyl acetate, dry over anhydrous sodium sulfate, concentrated, purified by prep-high pressure liquid chromatography to yield (R)-6-bromo-N-(3-(4,4,5,5-tetramethyl-1,3,2-dioxaborolan-2-yl)benzyl)-2,3,4,9-tetrahydro-1H-carbazol-1-amine.

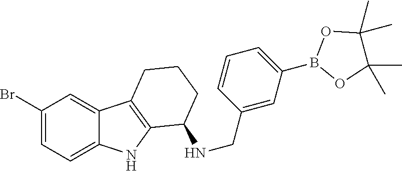 [SMILES: CC1(C)OB(c2cccc(CN[C@@H]3CCCc4c3[nH]c3ccc(Br)cc43)c2)OC1(C)C]

(R)-6-bromo-N-(3-(4,4,5,5-tetramethyl-1,3,2-dioxaborolan-2-yl)benzyl)-2,3,4,9-tetrahydro-1H-carbazol-1-amine. ¹H NMR (300 MHz, DMSO-d₆): δ10.87 (broad s, 1H), 7.66 (s, 1H), 7.57 (d, J=6.0 Hz, 1H), 7.49 (m, 2H), 7.33 (t, J=6.0 Hz, 1H), 7.24 (d, J=6.0 Hz, 1H), 7.08 (d, J=6.0 Hz, 1H), 3.83 (m, 3H), 2.54 (broad s, 2H), 1.96 (m, 2H), 1.68 (m, 2H) 1.26 (s, 12H). Method [1] retention time 2.11 min, MS(ESI positive) 248 and 250 (M-NHCH₂Ar), MS(ESI negative) 479 and 481 (M−H).

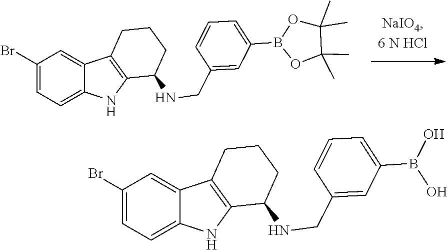 [SMILES: CC1(C)OB(c2cccc(CN[C@@H]3CCCc4c3[nH]c3ccc(Br)cc43)c2)OC1(C)C ; OB(O)c1cccc(CN[C@@H]2CCCc3c2[nH]c2ccc(Br)cc32)c1]

To a solution of (R)-6-bromo-N-(3-(4,4,5,5-tetramethyl-1,3,2-dioxaborolan-2-yl)benzyl)-2,3,4,9-tetrahydro-1H-carbazol-1-amine (100 mg) in tetrahydrofuran (10 mL), was 6 N hydrogen chloride (10 mL) added at room temperature. The reaction was stirred for 16 h. The reaction was monitored by liquid chromatography-mass spectrometry. Concentrated for the product, purified by prep-high pressure liquid chromatography to afford 41 mg of (R)-(3-(((6-bromo-2,3,4,9-tetrahydro-1H-carbazol-1-yl)amino)methyl)phenyl)boronic acid as white solid.

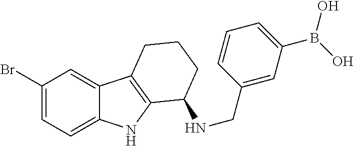 [SMILES: OB(O)c1cccc(CN[C@@H]2CCCc3c2[nH]c2ccc(Br)cc32)c1]

(R)-(3-(((6-bromo-2,3,4,9-tetrahydro-1H-carbazol-1-yl)amino)methyl)phenyl)boronic acid. ¹H NMR (300 MHz, DMSO-d₆): δ 11.05 (broad s, 1H), 9.05 (broad s, 2H), 8.15 (broad s, 2H), 7.89 (s, 1H), 7.82 (d, J=6.0 Hz, 1H), 7.68 (s, 1H), 7.54 (d, J=6.0 Hz, 1H), 7.39 (m, 2H), 7.25 (d, J=8.0, 1H), 4.65 (broad s, 1H), 4.36 (m, 1H), 4.26 (m, 1H), 2.71 (m, 1H), 2.58 (m, 1H), 2.19 (m, 1H), 2.11 (m, 1H), 2.00 (m, 1H), 1.82 (m, 1H). Method [1] retention time 1.84 min, MS(ESI positive) 248 and 250 (M-NHCH₂Ar), MS(ESI negative) 397 and 399 (M−H).

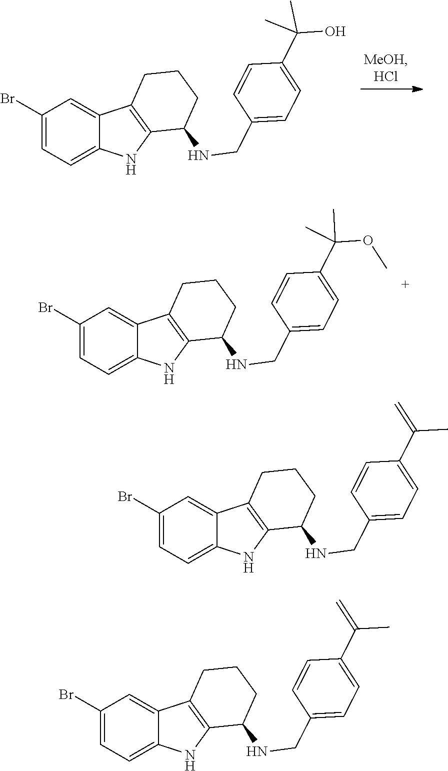 [SMILES: CC(C)(O)c1ccc(CN[C@@H]2CCCc3c2[nH]c2ccc(Br)cc32)cc1 ; COC(C)(C)c1ccc(CN[C@@H]2CCCc3c2[nH]c2ccc(Br)cc32)cc1 ; C=C(C)c1ccc(CN[C@@H]2CCCc3c2[nH]c2ccc(Br)cc32)cc1 ; C=C(C)c1ccc(CN[C@@H]2CCCc3c2[nH]c2ccc(Br)cc32)cc1]

(R)-6-bromo-N-(4-(prop-1-en-2-yl)benzyl)-2,3,4,9-tetrahydro-1H-carbazol-1-amine ¹H NMR (300 MHz, DMSO-d₆): δ 11.21 (m, 1H), 9.20 (broad s, 2H), 7.67 (s, 1H), 7.51 (m, 4H), 7.38 (d, J=6.0 Hz, 1H), 7.24 (d, J=6.0 Hz, 1H), 5.44 (s, 1H), 5.12 (s, 1H), 4.63 (broad s, 1H), 4.31 (m, 2H), 2.70 (m, 1H), 2.61 (m, 1H), 2.19 (m, 1H), 2.12 (m, 1H), 2.08 (s, 3H), 2.04 (m, 1H), 1.82 (m, 1H). Method [1] retention time 2.18 min, MS(ESI positive) 248 and 250 (M-NHCH₂Ar).

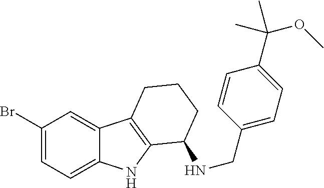 [SMILES: COC(C)(C)c1ccc(CN[C@@H]2CCCc3c2[nH]c2ccc(Br)cc32)cc1]

(R)-6-bromo-N-(4-(2-methoxypropan-2-yl)benzyl)-2,3,4,9-tetrahydro-1H-carbazol-1-amine. ¹H NMR (300 MHz, DMSO-d₆): δ 11.21 (m, 1H), 9.18 (broad s, 2H), 7.67 (s, 1H), 7.49 (d, J=6.0 Hz, 2H), 7.38 (m, 3H), 7.24 (d, J=6.0 Hz, 1H), 4.66 (broad s, 1H), 4.29 (m, 2H), 2.97 (s, 3H), 2.72 (m, 1H), 2.65 (m, 1H), 2.20 (m, 1H), 2.12 (m, 1H), 2.02 (m, 1H), 1.81 (m, 1H), 1.41 (s, 6H). Method [1] retention time 2.03 min, MS(ESI positive) 248 and 250 (M-NHCH₂Ar), MS(ESI negative) 425 and 427 (M−H).

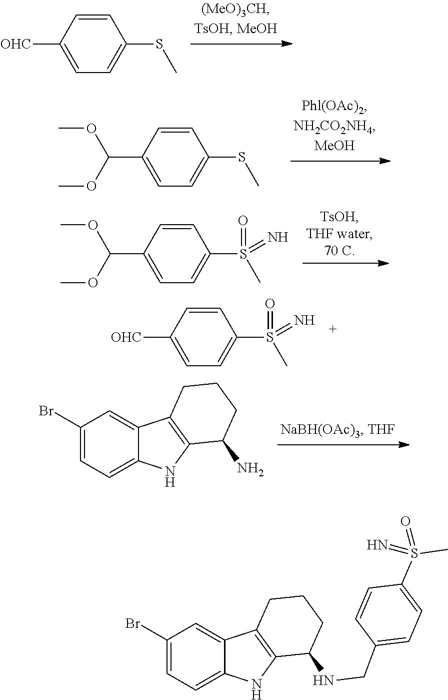

To a solution of 4-(methylthio)benzaldehyde (1.0 g) in methanol (40 mL), trimethoxymethane (1.82 g) and p-toluenesulfonic acid (125 mg) was added at room temperature. The reaction was stirred for 1 h. The reaction was monitored by thin layer chromatography, trace 1 leaving, stirred overnight. Then, 5M sodium methoxide in methanol (0.6 ml) was added at room temperature. The reaction was concentrated, purified by flash chromatography to afford 1.5 g of (4-(dimethoxymethyl)phenyl)(methyl)sulfane.

To a solution of (4-(dimethoxymethyl)phenyl)(methyl)sulfane (300 mg) in methanol (30 mL), (diacetoxyiodo)benzene (1.48 g), and ammonium carbamate (580 mg) was added at room temperature. The reaction was stirred for 1 h. The reaction was monitored by thin layer chromatography. The reaction was quenched with saturated aqueous sodium bicarbonate. Dichloromethane extracted with, washed with brine and water. The organic layer was dried with anhydrous sodium sulfate, concentrated under reduced pressure. The residue was purified with prep-thin layer chromatography to yield 200 mg of (4-(dimethoxymethyl)phenyl)(imino)(methyl)-λ⁶-sulfanone.

(4-(dimethoxymethyl)phenyl)(imino)(methyl)-λ⁶-sulfanone (200 mg) and p-toluenesulfonic acid (410 mg) in tetrahydrofuran (15 ml) and water (5 ml) at 70 C for 3 h. The solution was concentrated and purified to yield 200 mg of crude 4-(S-methylsulfonimidoyl)benzaldehyde.

To a solution of (R)-6-bromo-2,3,4,9-tetrahydro-1H-carbazol-1-amine (1000 mg) and 4-(S-methylsulfonimidoyl)benzaldehyde (960 mg) in tetrahydrofuran (100 mL), was added sodium triacetoxyborohydride (2.30 g) at room temperature. The mixture was stirred for 16 h. The reaction was monitored by liquid chromatography-mass spectrometry and thin layer chromatography. Quenched with saturated aqueous ammonium chloride, extracted with ethyl acetate, washed with brine, dried over anhydrous sodium sulfate, concentrated to yield 570 mg of (4-((((R)-6-bromo-2,3,4,9-tetrahydro-1H-carbazol-1-yl)amino)methyl)phenyl)(imino)(methyl)-λ⁶-sulfanone.

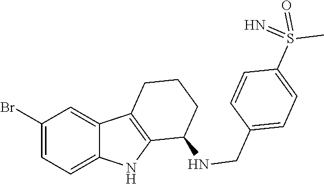 [SMILES: CS(=N)(=O)c1ccc(CN[C@@H]2CCCc3c2[nH]c2ccc(Br)cc32)cc1]

(4-((((R)-6-bromo-2,3,4,9-tetrahydro-1H-carbazol-1-yl)amino)methyl)phenyl)(imino)(methyl)-λ⁶-sulfanone. ¹H NMR (300 MHz, DMSO-d₆): δ 11.30 (m, 1H), 9.43 (broad s, 2H), 8.02 (d, J=6.0 Hz, 2H), 7.75 (d, J=6.0 Hz, 2H), 7.67 (s, 1H), 7.38 (d, J=6.0 Hz, 1H), 7.25 (d, J=6.0 Hz, 1H), 4.76 (broad s, 3H), 4.69 (s, 3H), 4.49 (d, J=9.0 Hz, 1H), 4.41 (d, J=9.0 Hz, 1H), 2.76 (m, 1H), 2.62 (m, 1H), 2.20 (m, 1H), 2.14 (m, 1H), 2.02 (m, 1H), 1.82 (m, 1H). Method [1] retention time 1.84 min, MS(ESI positive) 432 and 434 (M+H), MS(ESI negative) 430 and 432 (M−H).

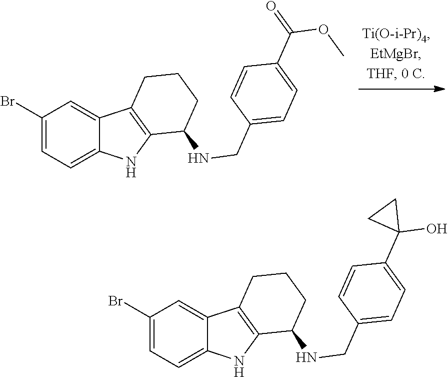 [SMILES: COC(=O)c1ccc(CN[C@@H]2CCCc3c2[nH]c2ccc(Br)cc32)cc1 ; OC1(c2ccc(CN[C@@H]3CCCc4c3[nH]c3ccc(Br)cc43)cc2)CC1]

To the solution of methyl (R)-4-(((6-bromo-2,3,4,9-tetrahydro-1H-carbazol-1-yl)amino)methyl)benzoate (200 mg) in the tetrahydrofuran (20 mL), titanium(IV)isopropoxide (0.2 ml), was added at room temperature. The reaction mixture was cooled to 0 C, ethylmagnesium bromide (2.72 ml) was added. The reaction mixture was stirred for 24 h. The reaction was monitored by thin layer chromatography and liquid chromatography-mass spectrometry.

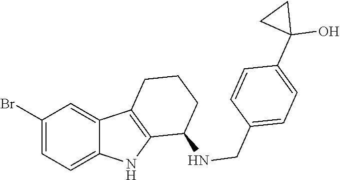 [SMILES: OC1(c2ccc(CN[C@@H]3CCCc4c3[nH]c3ccc(Br)cc43)cc2)CC1]

(R)-1-(4-(((6-bromo-2,3,4,9-tetrahydro-1H-carbazol-1-yl)amino)methyl)phenyl) cyclopropan-1-ol. ¹H NMR (300 MHz, DMSO-d₆): δ 11.14 (m, 1H), 9.11 (broad s, 2H), 7.67 (s, 1H), 7.37 (m, 3H), 7.24 (m, 3H), 5.95 (broad s, 1H), 4.62 (broad s, 1H), 4.25 (m, 2H), 2.72 (m, 1H), 2.62 (m, 1H), 2.18-2.00 (m, 3H), 1.81 (m, 1H), 1.10 (s, 2H), 0.92 (s, 2H). Method [1] retention time 1.97 min, MS(ESI positive) 248 and 250 (M-NHCH₂Ar), MS(ESI negative) 409 and 411 (M−H).

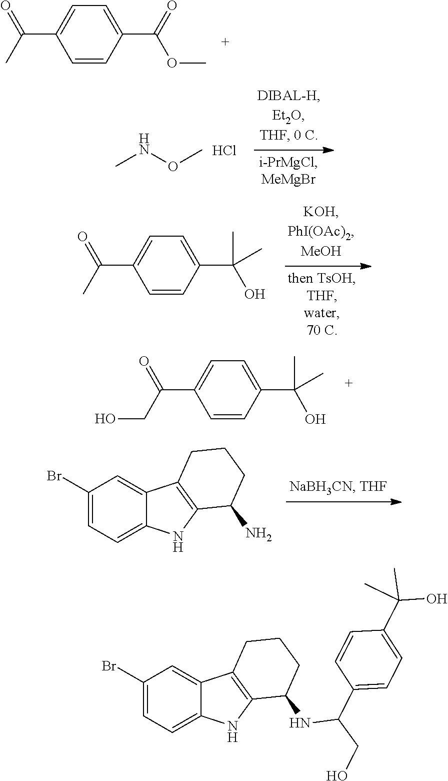

To a suspension solution of N,O-dimethylhydroxylamine hydrochloride (1.09 g) in diethyl ether (60 ml) and tetrahydrofuran (60 ml) under nitrogen atmosphere at 0° C., was added a solution 1 M diisobutylaluminium hydride in hexanes (11.2 mL). The mixture solution was stirred for 30 min allowing the temperature slowly to warm to room temperature. Then, cooled to 0° C., was added methyl 4-acetylbenzoate (1.0 g, 6.09 mmol, 1.0 eq in diethyl ether, 20 mL) and stirred for 2 h at room temperature. Then, a solution of 2M isopropyl magnesium chloride in tetrahydrofuran (31 mL) was added and stirred for 1 h at room temperature. Then, a solution of 3M methyl magnesium bromide in tetrahydrofuran (3.74 mL) was added to above mixture solution and stirred for 4 h at room temperature. The solution was monitored by liquid chromatography-mass spectrometry and thin layer chromatography. Quenched by saturated aqueous ammonium chloride. Extracted with ethyl acetate and washed with brine, dry over anhydrous sodium sulfate, concentrated and purified by flash chromatography to yield 200 mg of 1-(4-(2-hydroxypropan-2-yl)phenyl)ethan-1-one as a colorless oil.

To the solution of 1-(4-(2-hydroxypropan-2-yl)phenyl)ethan-1-one (100 mg) in methanol (3 mL), potassium hydroxide (142 mg) was added at OC. Then, (diacetoxyiodo)benzene (274 mg) was added at room temperature for 3 h. The crude product was mixed with p-toluenesulfonic acid (111 mg) in tetrahydrofuran (4.5 ml) and water (1.5 ml) at 70 C for 3 h. The reaction was monitored by thin layer chromatography and liquid chromatography-mass spectrometry to yield 70 mg of 2-hydroxy-1-(4-(2-hydroxypropan-2-yl)phenyl)ethan-1-one.

To a solution of (R)-6-bromo-2,3,4,9-tetrahydro-1H-carbazol-1-amine (86 mg) and 2-hydroxy-1-(4-(2-hydroxypropan-2-yl)phenyl)ethan-1-one (50 mg) in tetrahydrofuran (9 ml) and methanol (3 ml), was added sodium cyanoborohydride at room temperature. The mixture was heated to 70 C. Then glacial acetic acid 2 drops were added and stirred for 16 h. The reaction was monitored by liquid chromatography-mass spectrometry and thin layer chromatography to yield 2-(4-(1-(((R)-6-bromo-2,3,4,9-tetrahydro-1H-carbazol-1-yl)amino)-2-hydroxyethyl)phenyl)propan-2-ol.

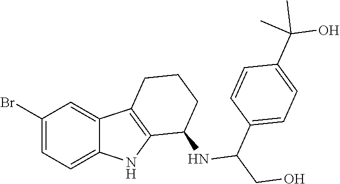 [SMILES: CC(C)(O)c1ccc(C(CO)N[C@@H]2CCCc3c2[nH]c2ccc(Br)cc32)cc1]

2-(4-(1-(((R)-6-bromo-2,3,4,9-tetrahydro-1H-carbazol-1-yl)amino)-2-hydroxyethyl)phenyl) propan-2-ol (2:1 mixture of diastereomers). ¹H NMR (300 MHz, DMSO-d₆): δ 11.20 (m, 1H), 9.22 (broad s, 2H), 7.63 (s, 1H), 7.50 (m, 4H), 7.38 (d, J=6.0 Hz, 1H), 7.23 (d, J=6.0 Hz, 1H), 5.62-4.8 (broad s, 1H), 4.62 (broad s, 1H), 4.51 (broad s, 0.33H), 4.39 (broad s, 0.67H), 3.86 (m, 2H), 2.47 (m, 2H), 2.12-2.00 (m, 3H), 1.69 (m, 1H), 1.38 (s, 6H). Method [1] retention time 1.89 min, MS(ESI positive) 443 and 445 (M+H), MS(ESI negative) 441 and 443 (M−H).

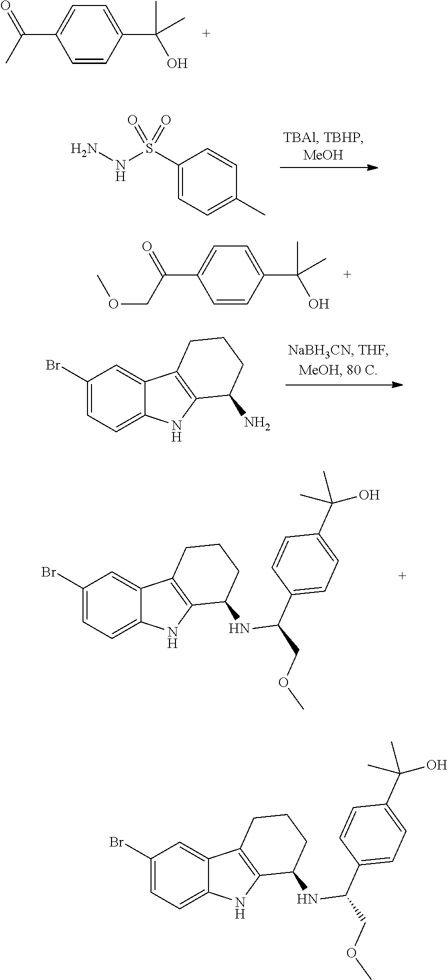

1-(4-(2-hydroxypropan-2-yl)phenyl)ethan-1-one (2.0 g), 4-methylbenzenesulfonohydrazide (2.1 g), tetrabutylammonium iodide (830 mg), and tert-butyl hydrogen peroxide (8.8 ml) in methanol (60 ml) was at 0 C to room temperature was stirred for 16 h. The material was purified to yield 800 mg of 1-(4-(2-hydroxypropan-2-yl)phenyl)-2-methoxyethan-1-one.

To a solution of (R)-6-bromo-2,3,4,9-tetrahydro-1H-carbazol-1-amine (152 mg) and 1-(4-(2-hydroxypropan-2-yl)phenyl)-2-methoxyethan-1-one (200 mg) in tetrahydrofuran (24 ml) and methanol (4 ml), was added sodium cyanoborohydride (150 mg) at room temperature. The mixture was heated to 80 C. Then glacial acetic acid 2 drops were added and stirred for 16 h. The reaction was monitored by liquid chromatography-mass spectrometry and thin layer chromatography to yield 16 mg of 2-(4-((S)-1-(((R)-6-bromo-2,3,4,9-tetrahydro-1H-carbazol-1-yl)amino)-2-methoxyethyl)phenyl)propan-2-ol and 6 mg of 2-(4-((R)-1-(((R)-6-bromo-2,3,4,9-tetrahydro-1H-carbazol-1-yl)amino)-2-methoxyethyl)phenyl)propan-2-ol.

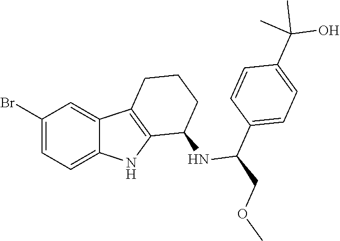 [SMILES: COC[C@@H](N[C@@H]1CCCc2c1[nH]c1ccc(Br)cc21)c1ccc(C(C)(C)O)cc1]

2-(4-((S)-1-(((R)-6-bromo-2,3,4,9-tetrahydro-1H-carbazol-1-yl)amino)-2-methoxyethyl)phenyl)propan-2-ol (S) Stereochemistry at benzylic position arbitrarily assigned. ¹H NMR (300 MHz, DMSO-d₆): δ 11.15 (s, 1H), 9.22 (broad s, 2H), 7.64 (s, 1H), 7.56 (d, J=6.0 Hz, 2H), 7.51 (d, J=6.0 Hz, 2H), 7.39 (d, J=6.0 Hz, 1H), 7.24 (d, J=6.0 Hz, 1H), 4.82 (broad s, 1H), 4.45 (broad s, 1H), 3.77 (m, 1H), 3.71 (m, 1H), 3.28 (s, 3H), 2.64 (m, 1H), 2.48 (m, 1H), 2.01 (m, 3H), 1.77 (m, 1H), 1.38 (s, 6H). Method [1] retention time 1.98 min, MS(ESI positive) 457 and 459 (M+H).

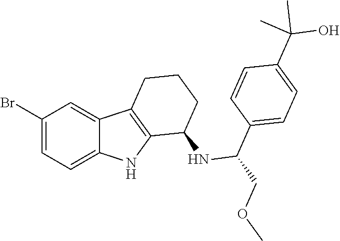 [SMILES: COC[C@H](N[C@@H]1CCCc2c1[nH]c1ccc(Br)cc21)c1ccc(C(C)(C)O)cc1]

2-(4-((R)-1-(((R)-6-bromo-2,3,4,9-tetrahydro-1H-carbazol-1-yl)amino)-2-methoxyethyl)phenyl)propan-2-ol (R) Stereochemistry at benzylic position arbitrarily assigned. ¹H NMR (300 MHz, DMSO-d₆): Q 11.19 (s, 1H), 9.36 (broad s, 2H), 7.63 (s, 1H), 7.51 (m, 4H), 7.38 (d, J=6.0 Hz, 1H), 7.22 (d, J=6.0 Hz, 1H), 4.82 (broad s, 1H), 4.34 (broad s, 1H), 3.82 (m, 1H), 3.72 (m, 1H), 3.30 (s, 3H), 2.48 (m, 2H), 2.13 (m, 1H), 2.01 (m, 2H), 1.70 (m, 1H), 1.38 (s, 6H). Method [1] retention time 2.03 min, MS(ESI positive) 457 and 459 (M+H).

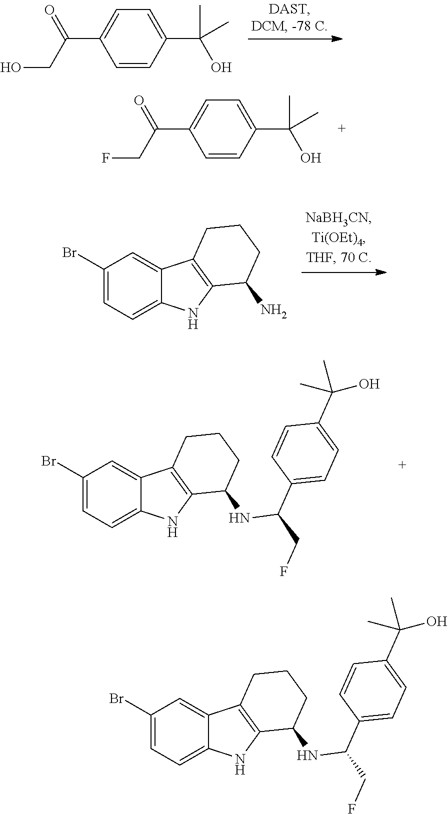

Diethylaminosulfur trifluoride (401 mg) was added to 1-(4-(2-hydroxypropan-2-yl)phenyl)ethan-1-one (430 mg) in dichloromethane (15 ml) at −78 C. After stirring for 4 h to room temperature, the reaction was purified by chromatography to yield 80 mg of 2-fluoro-1-(4-(2-hydroxypropan-2-yl)phenyl)ethan-1-one.

To a solution of (R)-6-bromo-2,3,4,9-tetrahydro-1H-carbazol-1-amine (120 mg) and 2-fluoro-1-(4-(2-hydroxypropan-2-yl)phenyl)ethan-1-one (90 mg) in tetrahydrofuran (24 ml) and titanium(IV)ethoxide (70 mg), was added sodium cyanoborohydride (150 mg) at room temperature. The mixture was heated to 70 C 6 h. The reaction was monitored by liquid chromatography-mass spectrometry and thin layer chromatography to yield 35 mg of 2-(4-((S)-1-(((R)-6-bromo-2,3,4,9-tetrahydro-1H-carbazol-1-yl)amino)-2-fluoroethyl)phenyl)propan-2-ol and 7.7 mg of 2-(4-((R)-1-(((R)-6-bromo-2,3,4,9-tetrahydro-1H-carbazol-1-yl)amino)-2-fluoroethyl)phenyl)propan-2-ol.

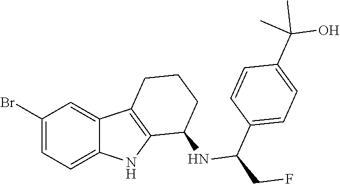 [SMILES: CC(C)(O)c1ccc([C@@H](CF)N[C@@H]2CCCc3c2[nH]c2ccc(Br)cc32)cc1]

2-(4-((S)-1-(((R)-6-bromo-2,3,4,9-tetrahydro-1H-carbazol-1-yl)amino)-2-fluoroethyl)phenyl)propan-2-ol (S) Stereochemistry at benzylic position arbitrarily assigned. ¹H NMR (300 MHz, CD₃OD): δ 7.54-7.61 (m, 5H), 7.31 (d, J=9.0 Hz, 1H), 7.25 (d, J=9.0 Hz, 1H), 5.00 (m, 3H), 4.63 (broad s, 1H), 2.81 (m, 1H), 2.68 (m, 1H), 2.14 (m, 2H), 2.01 (m, 1H), 1.95 (m, 1H), 1.50 (s, 6H). Method [1] retention time 1.72 min, MS(ESI positive) 248 and 250 (M-NHCH₂Ar), MS(ESI negative) 443 and 445 (M−H).

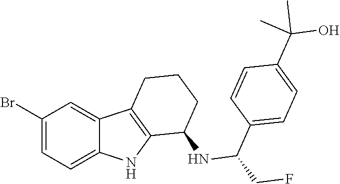 [SMILES: CC(C)(O)c1ccc([C@H](CF)N[C@@H]2CCCc3c2[nH]c2ccc(Br)cc32)cc1]

2-(4-((R)-1-(((R)-6-bromo-2,3,4,9-tetrahydro-1H-carbazol-1-yl)amino)-2-fluoroethyl)phenyl)propan-2-ol (R) Stereochemistry at benzylic position arbitrarily assigned. ¹H NMR (300 MHz, CD₃OD): δ 7.55-7.64 (m, 5H), 7.30 (d, J=6.0 Hz, 1H), 7.24 (d, J=6.0 Hz, 1H), 5.00 (m, 3H), 4.47 (m, 1H), 2.72 (m, 2H), 2.21 (m, 2H), 2.06 (m, 1H), 1.86 (m, 1H), 1.51 (s, 6H). Method [1] retention time 1.78 min, MS(ESI positive) 248 and 250 (M-NHCH₂Ar), MS(ESI negative) 443 and 445 (M−H).

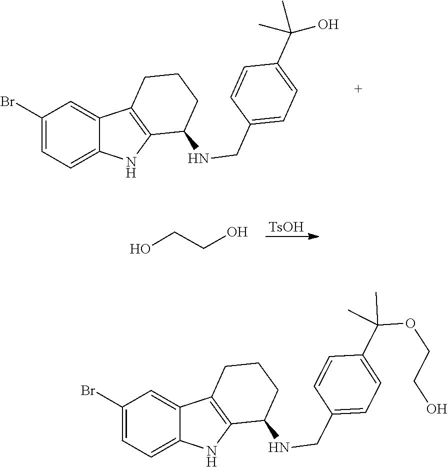

(R)-2-(4-(((6-bromo-2,3,4,9-tetrahydro-1H-carbazol-1-yl)amino)methyl)phenyl)propan-2-ol (200 mg) and p-toluenesulfonic acid (46 mg) in ethane-1,2-diol (2 ml) was stirred for 5 days. The mixture was purified by chromatography to yield 52 mg of (R)-2-((2-(4-(((6-bromo-2,3,4,9-tetrahydro-1H-carbazol-1-yl)amino)methyl)phenyl)propan-2-yl)oxy)ethan-1-ol.

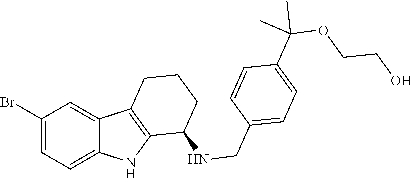 [SMILES: CC(C)(OCCO)c1ccc(CN[C@@H]2CCCc3c2[nH]c2ccc(Br)cc32)cc1]

(R)-2-((2-(4-(((6-bromo-2,3,4,9-tetrahydro-1H-carbazol-1-yl)amino)methyl)phenyl)propan-2-yl)oxy)ethan-1-ol. ¹H NMR (300 MHz, DMSO-d₆): δ 11.19 (s, 1H), 9.19 (broad s, 2H), 7.67 (s, 1H), 7.49 (m, 4H), 7.38 (d, J=6.0 Hz, 1H), 7.24 (d, J=6.0 Hz, 1H), 4.66 (broad s, 1H), 4.30 (s, 2H), 3.44 (t, J=6.0 Hz, 2H), 3.11 (t, J=6.0 Hz, 2H), 2.70 (m, 1H), 2.59 (m, 1H), 2.20-2.02 (m, 3H), 1.81 (m, 1H), 1.42 (s, 6H). Method [1] retention time 1.95 min, MS(ESI positive) 479 and 481 (M+Na), MS(ESI negative) 455 and 457 (M−H).

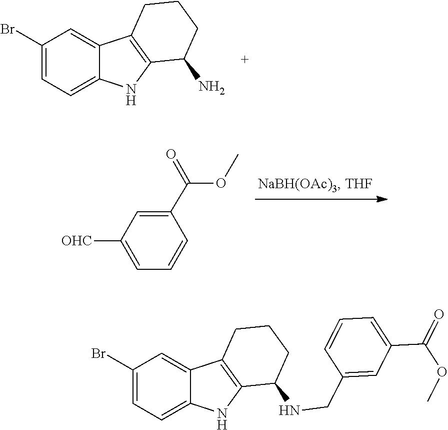 [SMILES: N[C@@H]1CCCc2c1[nH]c1ccc(Br)cc21 ; COC(=O)c1cccc(C=O)c1 ; COC(=O)c1cccc(CN[C@@H]2CCCc3c2[nH]c2ccc(Br)cc32)c1]

To a solution of (R)-6-bromo-2,3,4,9-tetrahydro-1H-carbazol-1-amine (320 mg) and methyl 3-formylbenzoate (300 mg) in tetrahydrofuran (30 mL), was added sodium triacetoxyborohydride (775 mg) at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-high pressure liquid chromatography to afford 240 mg of methyl (R)-3-(((6-bromo-2,3,4,9-tetrahydro-1H-carbazol-1-yl)amino)methyl)benzoate.

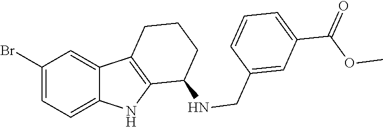 [SMILES: COC(=O)c1cccc(CN[C@@H]2CCCc3c2[nH]c2ccc(Br)cc32)c1]

Methyl (R)-3-(((6-bromo-2,3,4,9-tetrahydro-1H-carbazol-1-yl)amino)methyl)benzoate ¹H NMR (300 MHz, DMSO-d₆): δ 11.21 (s, 1H), 9.28 (broad s, 2H), 8.17 (s, 1H), 7.96 (d, J=6.0 Hz, 1H), 7.78 (d, J=6.0 Hz, 1H), 7.67 (s, 1H), 7.56 (t, J=6.0 Hz, 1H), 7.38 (d, J=6.0 Hz, 1H), 7.24 (d, J=6.0 Hz, 1H), 4.67 (broad s, 1H), 4.40 (m, 2H), 3.80 (s, 3H), 2.73 (m, 1H), 2.60 (m, 1H), 2.20-2.03 (m, 3H), 1.83 (m, 1H). Method [1] retention time 1.98 min, MS(ESI positive) 248 and 250 (M+H), MS(ESI negative) 411 and 413 (M−H).

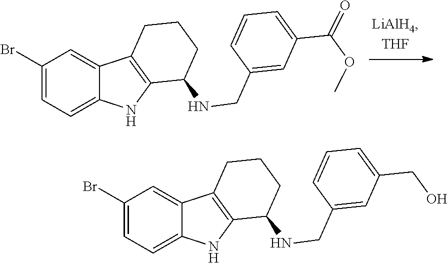 [SMILES: COC(=O)c1cccc(CN[C@@H]2CCCc3c2[nH]c2ccc(Br)cc32)c1 ; OCc1cccc(CN[C@@H]2CCCc3c2[nH]c2ccc(Br)cc32)c1]

To a solution of methyl (R)-3-(((6-bromo-2,3,4,9-tetrahydro-1H-carbazol-1-yl)amino)methyl)benzoate (150 mg) in tetrahydrofuran (20 mL), lithium aluminium deuteride (28 mg) was added at −40 C. The reaction was stirred for 3 h at room temperature. The reaction was monitored by liquid chromatography-mass spectrometry and thin layer chromatography.

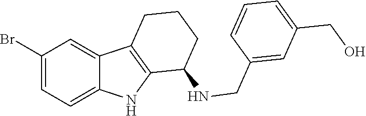 [SMILES: OCc1cccc(CN[C@@H]2CCCc3c2[nH]c2ccc(Br)cc32)c1]

(R)-(3-(((6-bromo-2,3,4,9-tetrahydro-1H-carbazol-1-yl)amino)methyl)phenyl)methanol. ¹H NMR (300 MHz, DMSO-d₆): δ 11.22 (broad s, 1H), 9.23 (broad s, 2H), 7.67 (s, 1H), 7.48 (s, 1H), 7.38 (m, 4H), 7.24 (d, J=6.0 Hz, 1H), 4.65 (broad s, 1H), 4.50 (s, 2H), 4.31 (m, 2H), 2.70 (m, 1H), 2.61 (m, 1H), 2.20-2.02 (m, 3H), 1.80 (m, 1H). Method [1] retention time 1.50 min, MS(ESI positive) 248 and 250 (M-NHCH₂Ar), MS(ESI negative) 383 and 385 (M−H).

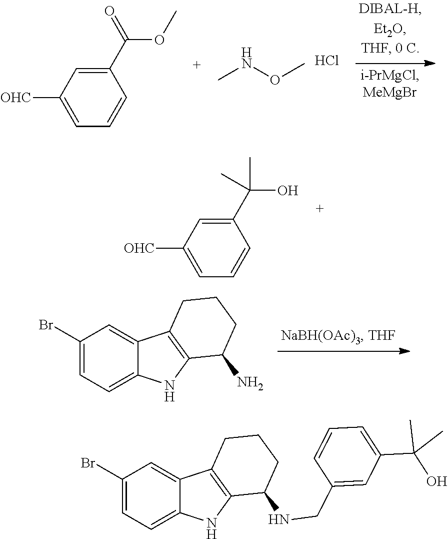

To a suspension solution of N,O-dimethylhydroxylamine hydrochloride (356 mg) in diethyl ether (70 ml) and tetrahydrofuran (30 ml) under nitrogen atmosphere at 0° C., was added a solution 1 M diisobutylaluminium hydride in hexanes (6.85 mL). The mixture solution was stirred for 30 min allowing the temperature slowly to warm to room temperature. Then, cooled to 0° C., was added methyl 3-formylbenzoate (500 mg, 6.09 mmol in diethyl ether 20 mL) and stirred for 2 h at room temperature. Then, a solution of 2M isopropyl magnesium chloride in tetrahydrofuran (1.82 mL) was added and stirred for 1 h at room temperature. Then, a solution of 3M methyl magnesium bromide in tetrahydrofuran (6.85 mL) was added to above mixture solution and stirred for 4 h at room temperature. The solution was monitored by liquid chromatography-mass spectrometry and thin layer chromatography. Quenched by saturated aqueous ammonium chloride. Extracted with ethyl acetate and washed with brine, dry over anhydrous sodium sulfate, concentrated and purified by flash chromatography to yield 200 mg of 3-(2-hydroxypropan-2-yl)benzaldehyde.

To a solution of (R)-6-bromo-2,3,4,9-tetrahydro-1H-carbazol-1-amine (300 mg) and 3-(2-hydroxypropan-2-yl)benzaldehyde (150 mg) in tetrahydrofuran (10 mL), was added sodium triacetoxyborohydride (525 mg) at room temperature. The mixture was stirred for 16 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-thin layer chromatography to afford 250 mg of (R)-2-(3-(((6-bromo-2,3,4,9-tetrahydro-1H-carbazol-1-yl)amino)methyl)phenyl)propan-2-ol as white solid.

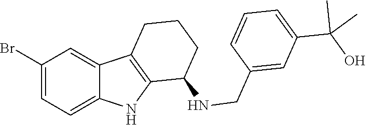 [SMILES: CC(C)(O)c1cccc(CN[C@@H]2CCCc3c2[nH]c2ccc(Br)cc32)c1]

(R)-2-(3-(((6-bromo-2,3,4,9-tetrahydro-1H-carbazol-1-yl)amino)methyl) phenyl)propan-2-ol. ¹H NMR (300 MHz, DMSO-d₆): δ 11.10 (s, 1H), 9.14 (broad s, 2H), 7.68 (s, 1H), 7.48 (s, 1H), 7.46 (m, 1H), 7.36 (d, J=6.0 Hz, 1H), 7.33 (m, 2H), 7.25 (d, J=6.0 Hz, 1H), 5.06 (broad s, 1H), 4.66 (broad s, 1H), 4.34 (m, 1H), 4.28 (m, 1H), 2.71 (m, 1H), 2.60 (m, 1H), 2.20-2.02 (m, 3H), 1.81 (m, 1H), 1.40 (s, 6H). Method [1] retention time 1.93 min, MS(ESI positive) 248 and 250 (M-NHCH₂Ar), MS(ESI negative) 411 and 413 (M−H).

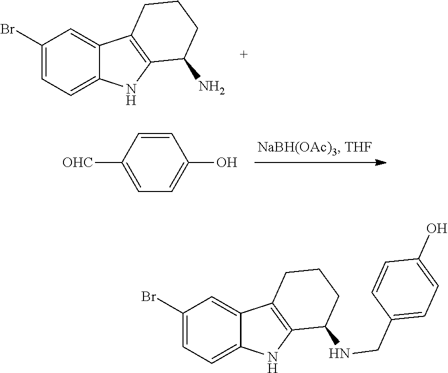

To a solution of (R)-6-bromo-2,3,4,9-tetrahydro-1H-carbazol-1-amine (300 mg) and 4-hydroxybenzaldehyde (150 mg) in tetrahydrofuran (3 mL), was added sodium triacetoxyborohydride (525 mg) at room temperature. The mixture was stirred for 48 h. The reaction was monitored by liquid chromatography-mass spectrometry and thin layer chromatography. Quenched with saturated aqueous ammonium chloride, extracted with ethyl acetate, dried over anhydrous sodium sulfate, concentrated, purified by prep-thin layer chromatography to afford 215 mg of (R)-4-(((6-bromo-2,3,4,9-tetrahydro-1H-carbazol-1-yl)amino)methyl)phenol as white solid.

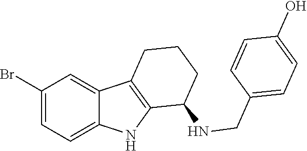 [SMILES: Oc1ccc(CN[C@@H]2CCCc3c2[nH]c2ccc(Br)cc32)cc1]

(R)-4-(((6-bromo-2,3,4,9-tetrahydro-1H-carbazol-1-yl)amino)methyl)phenol. ¹H NMR (300 MHz, DMSO-d₆): δ 11.06 (s, 1H), 9.69 (s, 1H), 8.94 (broad s, 2H), 7.67 (s, 1H), 7.38 (d, J=6.0 Hz, 1H), 7.31 (d, J=6.0 Hz, 1H), 7.24 (d, J=6.0 Hz, 1H), 6.78 (d, J=6.0 Hz, 2H), 4.59 (broad s, 1H), 4.18 (m, 2H), 2.72 (m, 1H), 2.59 (m, 1H), 2.16-1.99 (m, 3H), 1.81 (m, 1H). Method [1] retention time 1.92 min, MS(ESI positive) 248 and 250 (M-NHCH₂Ar), MS(ESI negative) 369 and 371 (M−H).

TABLE 2
Exemplary Compounds

| Compound # | Structure | pKa¹ | LogD¹ | tPSA¹ |
|---|---|---|---|---|
| 1 | 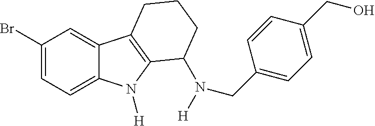 [SMILES: OCc1ccc(CNC2CCCc3c2[nH]c2ccc(Br)cc32)cc1] | 9.0 | 1.9 | 48 |
| 2 | 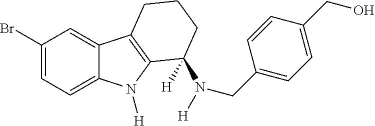 [SMILES: OCc1ccc(CN[C@@H]2CCCc3c2[nH]c2ccc(Br)cc32)cc1] | 9.0 | 1.9 | 48 |
| 3 | 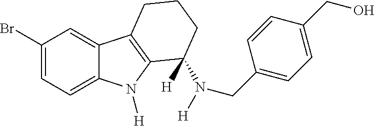 [SMILES: OCc1ccc(CN[C@H]2CCCc3c2[nH]c2ccc(Br)cc32)cc1] | 9.0 | 1.9 | 48 |
| 4 | 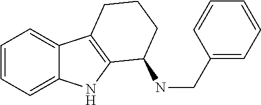 [SMILES: c1ccc(CN[C@@H]2CCCc3c2[nH]c2ccccc32)cc1] | 9.6 | 1.8 | 28 |
| 5 | 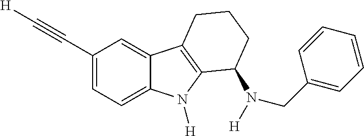 [SMILES: C#Cc1ccc2[nH]c3c(c2c1)CCC[C@H]3NCc1ccccc1] | 9.4 | 1.9 | 28 |
| 6 | 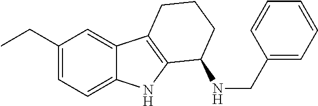 [SMILES: CCc1ccc2[nH]c3c(c2c1)CCC[C@H]3NCc1ccccc1] | 9.5 | 2.3 | 28 |
| 7 | 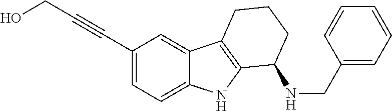 [SMILES: OCC#Cc1ccc2[nH]c3c(c2c1)CCC[C@H]3NCc1ccccc1] | 9.3 | 1.7 | 48 |
| 8 | 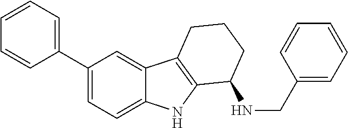 [SMILES: c1ccc(CN[C@@H]2CCCc3c2[nH]c2ccc(-c4ccccc4)cc32)cc1] | 9.5 | 3.1 | 28 |
| 9 | 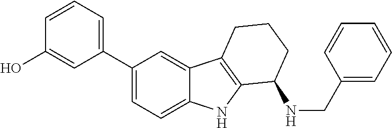 [SMILES: Oc1cccc(-c2ccc3[nH]c4c(c3c2)CCC[C@H]4NCc2ccccc2)c1] | 9.5 | 2.6 | 48 |
| 10 | 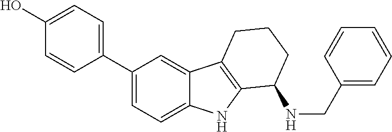 [SMILES: Oc1ccc(-c2ccc3[nH]c4c(c3c2)CCC[C@H]4NCc2ccccc2)cc1] | 9.5 | 2.6 | 48 |
| 11 | 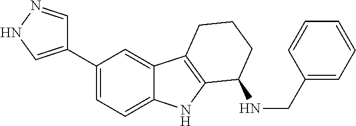 [SMILES: c1ccc(CN[C@@H]2CCCc3c2[nH]c2ccc(-c4cn[nH]c4)cc32)cc1] | 9.4 | 1.7 | 57 |
| 12 | 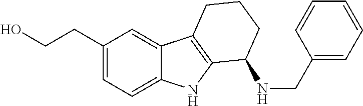 [SMILES: OCCc1ccc2[nH]c3c(c2c1)CCC[C@H]3NCc1ccccc1] | 9.5 | 1.1 | 48 |
| 13 | 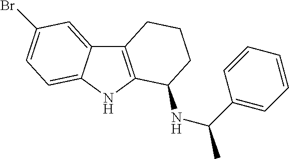 [SMILES: C[C@@H](N[C@@H]1CCCc2c1[nH]c1ccc(Br)cc21)c1ccccc1] | 9.2 | 3.0 | 28 |
| 14 | 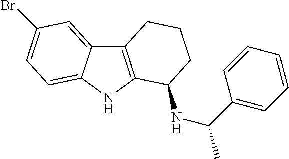 [SMILES: C[C@H](N[C@@H]1CCCc2c1[nH]c1ccc(Br)cc21)c1ccccc1] | 9.2 | 3.0 | 28 |
| 15 | 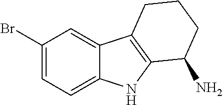 [SMILES: N[C@@H]1CCCc2c1[nH]c1ccc(Br)cc21] | 10.3 | −0.3 | 42 |
| 16 | 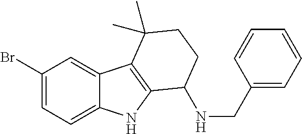 [SMILES: CC1(C)CCC(NCc2ccccc2)c2[nH]c3ccc(Br)cc3c21] | 9.2 | 3.5 | 28 |
| 17 | 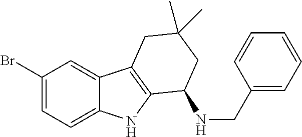 [SMILES: CC1(C)Cc2c([nH]c3ccc(Br)cc23)[C@H](NCc2ccccc2)C1] | 9.2 | 3.4 | 28 |
| 18 | 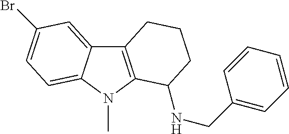 [SMILES: Cn1c2c(c3cc(Br)ccc31)CCCC2NCc1ccccc1] | 9.3 | 3.6 | 17 |
| 19 | 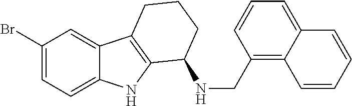 [SMILES: Brc1ccc2[nH]c3c(c2c1)CCC[C@H]3NCc1cccc2ccccc12] | 8.9 | 4.0 | 28 |
| 20 | 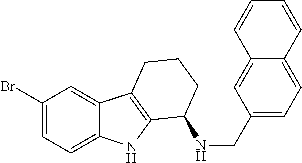 [SMILES: Brc1ccc2[nH]c3c(c2c1)CCC[C@H]3NCc1ccc2ccccc2c1] | 9.0 | 4.0 | 28 |
| 21 | 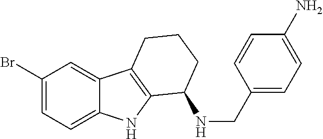 [SMILES: Nc1ccc(CN[C@@H]2CCCc3c2[nH]c2ccc(Br)cc32)cc1] | 9.7 | 1.5 | 54 |
| 22 | 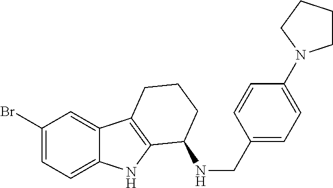 [SMILES: Brc1ccc2[nH]c3c(c2c1)CCC[C@H]3NCc1ccc(N2CCCC2)cc1] | 9.8 | 2.4 | 31 |
| 23 | 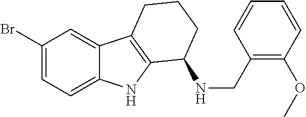 [SMILES: COc1ccccc1CN[C@@H]1CCCc2c1[nH]c1ccc(Br)cc21] | 9.2 | 2.7 | 37 |
| 24 | 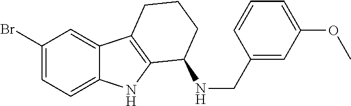 [SMILES: COc1cccc(CN[C@@H]2CCCc3c2[nH]c2ccc(Br)cc32)c1] | 9.1 | 2.7 | 37 |
| 25 | 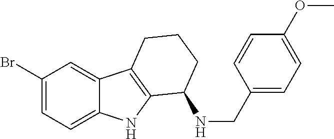 [SMILES: COc1ccc(CN[C@@H]2CCCc3c2[nH]c2ccc(Br)cc32)cc1] | 9.6 | 2.3 | 37 |
| 26 | 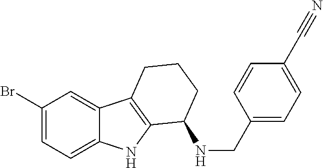 [SMILES: N#Cc1ccc(CN[C@@H]2CCCc3c2[nH]c2ccc(Br)cc32)cc1] | 8.5 | 3.1 | 52 |
| 27 | 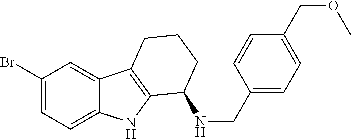 [SMILES: COCc1ccc(CN[C@@H]2CCCc3c2[nH]c2ccc(Br)cc32)cc1] | 9.0 | 2.9 | 37 |
| 28 | 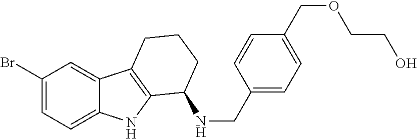 [SMILES: OCCOCc1ccc(CN[C@@H]2CCCc3c2[nH]c2ccc(Br)cc32)cc1] | 9.0 | 2.4 | 57 |
| 29 | 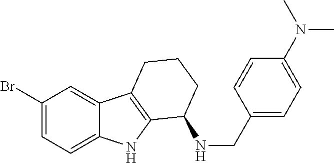 [SMILES: CN(C)c1ccc(CN[C@@H]2CCCc3c2[nH]c2ccc(Br)cc32)cc1] | 9.8 | 2.5 | 32 |
| 30 | 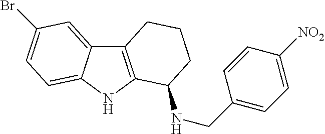 [SMILES: O=[N+]([O-])c1ccc(CN[C@@H]2CCCc3c2[nH]c2ccc(Br)cc32)cc1] | 8.3 | 3.5 | 77 |
| 31 | 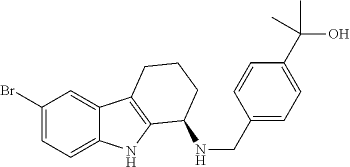 [SMILES: CC(C)(O)c1ccc(CN[C@@H]2CCCc3c2[nH]c2ccc(Br)cc32)cc1] | 9.2 | 2.5 | 48 |
| 32 | 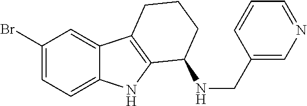 [SMILES: Brc1ccc2[nH]c3c(c2c1)CCC[C@H]3NCc1cccnc1] | 8.1 | 2.6 | 41 |
| 33 | 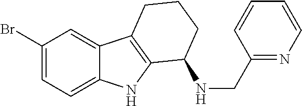 [SMILES: Brc1ccc2[nH]c3c(c2c1)CCC[C@H]3NCc1ccccn1] | 7.8 | 3.3 | 41 |
| 34 | 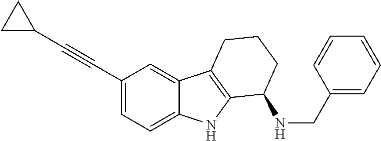 [SMILES: C(#CC1CC1)c1ccc2[nH]c3c(c2c1)CCC[C@H]3NCc1ccccc1] | 9.3 | 2.8 | 28 |
| 35 | 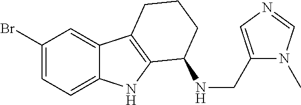 [SMILES: Cn1cncc1CN[C@@H]1CCCc2c1[nH]c1ccc(Br)cc21] | 9.2 | 0.9 | 46 |
| 36 | 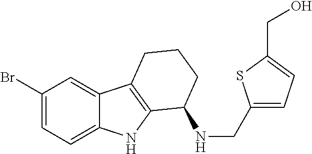 [SMILES: OCc1ccc(CN[C@@H]2CCCc3c2[nH]c2ccc(Br)cc32)s1] | 8.7 | 1.9 | 76 |
| 37 | 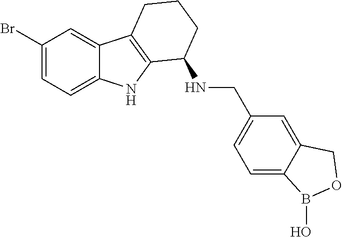 [SMILES: OB1OCc2cc(CN[C@@H]3CCCc4c3[nH]c3ccc(Br)cc43)ccc21] | 9.1; 6.9 (OH) | 1.4 | 57 |
| 38 | 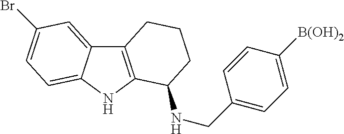 [SMILES: OB(O)c1ccc(CN[C@@H]2CCCc3c2[nH]c2ccc(Br)cc32)cc1] | 8.9; 8.4 (OH) | 2.3 | 68 |
| 39 | 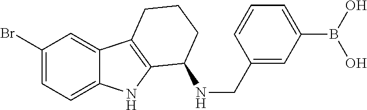 [SMILES: OB(O)c1cccc(CN[C@@H]2CCCc3c2[nH]c2ccc(Br)cc32)c1] | 8.8; 8.4 (OH) | 2.3 | 68 |
| 40 | 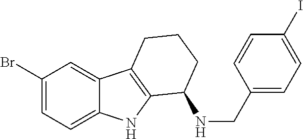 [SMILES: Brc1ccc2[nH]c3c(c2c1)CCC[C@H]3NCc1ccc(I)cc1] | 8.8 | 3.8 | 28 |
| 41 | 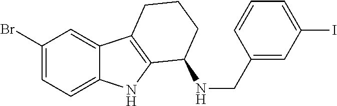 [SMILES: Brc1ccc2[nH]c3c(c2c1)CCC[C@H]3NCc1cccc(I)c1] | 8.7 | 3.9 | 28 |
| 42 | 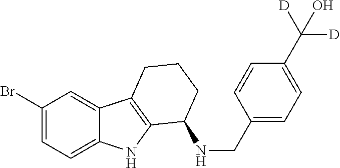 [SMILES: [2H]C([2H])(O)c1ccc(CN[C@@H]2CCCc3c2[nH]c2ccc(Br)cc32)cc1] | 9.0 | 1.9 | 48 |
| 43 | 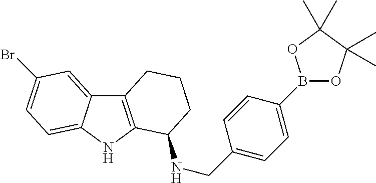 [SMILES: CC1(C)OB(c2ccc(CN[C@@H]3CCCc4c3[nH]c3ccc(Br)cc43)cc2)OC1(C)C] | 8.9 | 3.2 | 46 |
| 44 | 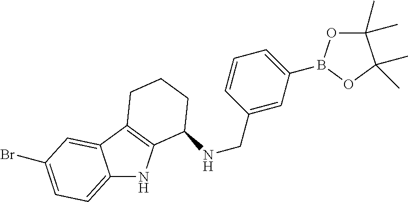 [SMILES: CC1(C)OB(c2cccc(CN[C@@H]3CCCc4c3[nH]c3ccc(Br)cc43)c2)OC1(C)C] | 9.0 | 3.0 | 46 |
| 45 | 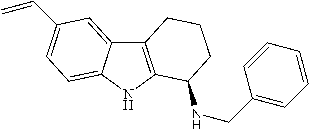 [SMILES: C=Cc1ccc2[nH]c3c(c2c1)CCC[C@H]3NCc1ccccc1] | 9.5 | 2.4 | 28 |
| 46 | 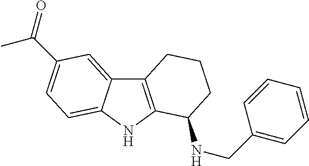 [SMILES: CC(=O)c1ccc2[nH]c3c(c2c1)CCC[C@H]3NCc1ccccc1] | 9.3 | 1.4 | 45 |
| 47 | 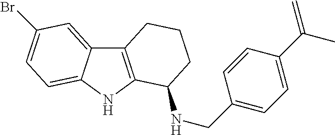 [SMILES: C=C(C)c1ccc(CN[C@@H]2CCCc3c2[nH]c2ccc(Br)cc32)cc1] | 9.0 | 3.7 | 28 |
| 48 | 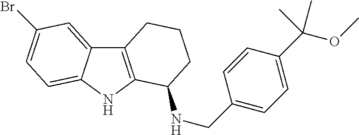 [SMILES: COC(C)(C)c1ccc(CN[C@@H]2CCCc3c2[nH]c2ccc(Br)cc32)cc1] | 9.0 | 3.2 | 37 |
| 49 | 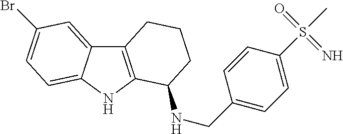 [SMILES: CS(=N)(=O)c1ccc(CN[C@@H]2CCCc3c2[nH]c2ccc(Br)cc32)cc1] | 8.3 | 2.0 | 70 |
| 50 | | 9.2 | 2.1 | 48 |
| 51 | 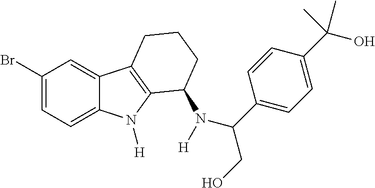 [SMILES: CC(C)(O)c1ccc(C(CO)N[C@@H]2CCCc3c2[nH]c2ccc(Br)cc32)cc1] | 8.2 | 2.7 | 57 |
| 52* | 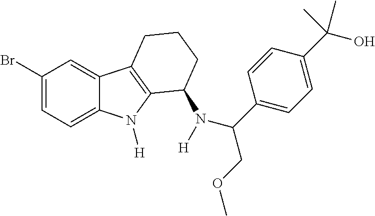 [SMILES: COCC(N[C@@H]1CCCc2c1[nH]c1ccc(Br)cc21)c1ccc(C(C)(C)O)cc1] | 8.3 | 3.4 | 68 |
| 53* | 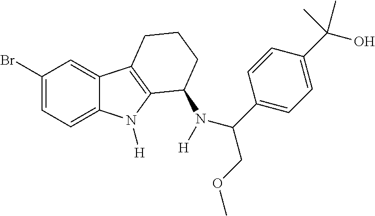 [SMILES: COCC(N[C@@H]1CCCc2c1[nH]c1ccc(Br)cc21)c1ccc(C(C)(C)O)cc1] | 8.3 | 3.4 | 68 |
| 54* | 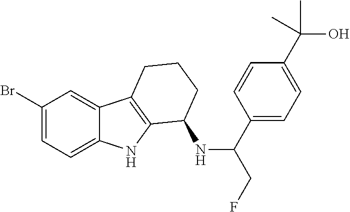 [SMILES: CC(C)(O)c1ccc(C(CF)N[C@@H]2CCCc3c2[nH]c2ccc(Br)cc32)cc1] | 7.3 | 4.2 | 48 |
| 55* | 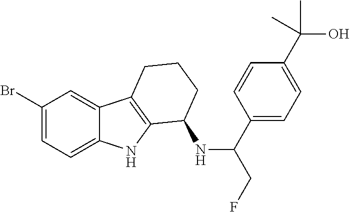 [SMILES: CC(C)(O)c1ccc(C(CF)N[C@@H]2CCCc3c2[nH]c2ccc(Br)cc32)cc1] | 7.3 | 4.2 | 48 |
| 56 | 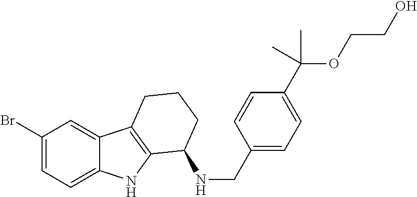 [SMILES: CC(C)(OCCO)c1ccc(CN[C@@H]2CCCc3c2[nH]c2ccc(Br)cc32)cc1] | 9.2 | 2.8 | 57 |
| 57 | 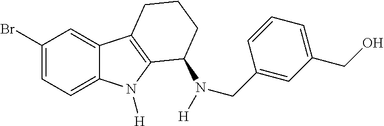 [SMILES: OCc1cccc(CN[C@@H]2CCCc3c2[nH]c2ccc(Br)cc32)c1] | 9.0 | 2.0 | 48 |
| 58 | 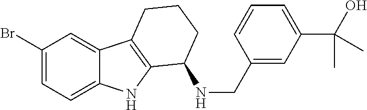 [SMILES: CC(C)(O)c1cccc(CN[C@@H]2CCCc3c2[nH]c2ccc(Br)cc32)c1] | 9.1 | 2.6 | 48 |
| 59 | 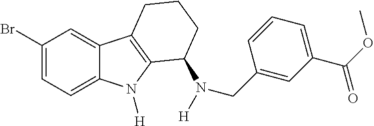 [SMILES: COC(=O)c1cccc(CN[C@@H]2CCCc3c2[nH]c2ccc(Br)cc32)c1] | 8.7 | 3.3 | 54 |
| 60 | 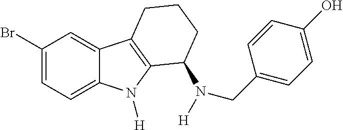 [SMILES: Oc1ccc(CN[C@@H]2CCCc3c2[nH]c2ccc(Br)cc32)cc1] | 10.0 (OH); 9.4 | 1.9 | 48 |
| 61 | 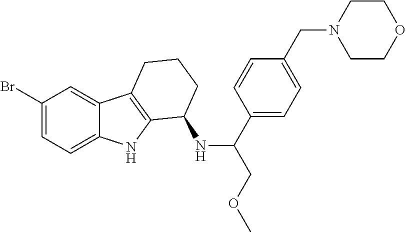 [SMILES: COCC(N[C@@H]1CCCc2c1[nH]c1ccc(Br)cc21)c1ccc(CN2CCOCC2)cc1] | 8.3; 6.3 | 3.0 | 50 |
| 62 | 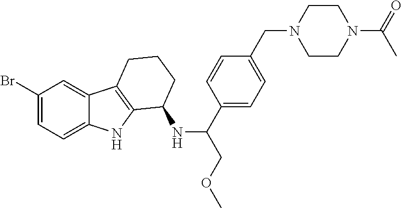 [SMILES: COCC(N[C@@H]1CCCc2c1[nH]c1ccc(Br)cc21)c1ccc(CN2CCN(C(C)=O)CC2)cc1] | 8.2; 6.0 | 2.9 | 61 |
| 63 | 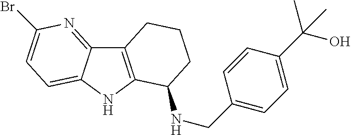 [SMILES: CC(C)(O)c1ccc(CN[C@@H]2CCCc3c2[nH]c2ccc(Br)nc32)cc1] | 8.5 | 2.0 | 61 |
| 64 | 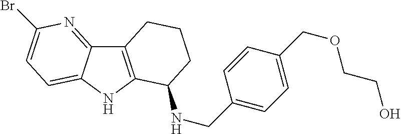 [SMILES: OCCOCc1ccc(CN[C@@H]2CCCc3c2[nH]c2ccc(Br)nc32)cc1] | 8.4 | 2.1 | 70 |
| 65 | 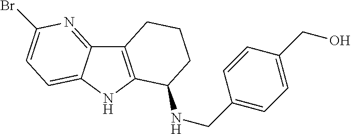 [SMILES: OCc1ccc(CN[C@@H]2CCCc3c2[nH]c2ccc(Br)nc32)cc1] | 8.4 | 1.4 | 61 |
| 66 | 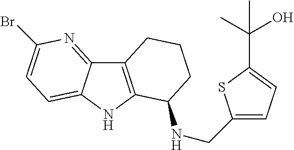 [SMILES: CC(C)(O)c1ccc(CN[C@@H]2CCCc3c2[nH]c2ccc(Br)nc32)s1] | 8.0 | 2.2 | 89 |
| 67 | 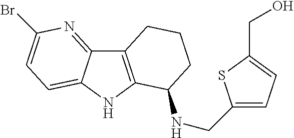 [SMILES: OCc1ccc(CN[C@@H]2CCCc3c2[nH]c2ccc(Br)nc32)s1] | 8.1 | 1.5 | 89 |
| 68 | 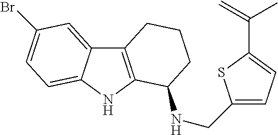 [SMILES: C=C(C)c1ccc(CN[C@@H]2CCCc3c2[nH]c2ccc(Br)cc32)s1] | 8.5 | 4.0 | 56 |
| 69 | 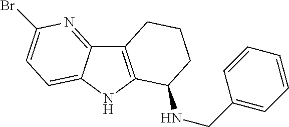 [SMILES: Brc1ccc2[nH]c3c(c2n1)CCC[C@H]3NCc1ccccc1] | 8.6 | 2.3 | 41 |
| 70 | 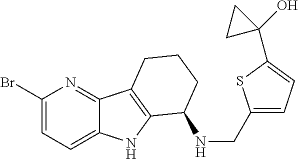 [SMILES: OC1(c2ccc(CN[C@@H]3CCCc4c3[nH]c3ccc(Br)nc43)s2)CC1] | 8.0 | 2.0 | 89 |
| 71 | 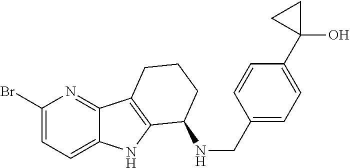 [SMILES: OC1(c2ccc(CN[C@@H]3CCCc4c3[nH]c3ccc(Br)nc43)cc2)CC1] | 8.5 | 1.7 | 61 |
| 72 | 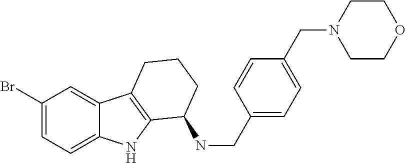 [SMILES: Brc1ccc2[nH]c3c(c2c1)CCC[C@H]3NCc1ccc(CN2CCOCC2)cc1] | 9.0; 6.4 | 2.0 | 40 |
| 73 | 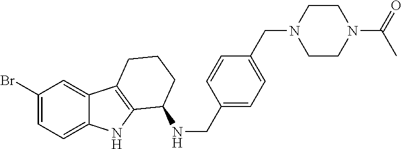 [SMILES: CC(=O)N1CCN(Cc2ccc(CN[C@@H]3CCCc4c3[nH]c3ccc(Br)cc43)cc2)CC1] | 9.2; 6.0 | 1.8 | 51 |

¹pKa, logD at pH 7.4 and tPSA are calculated using ACD/Labs Percepta Version 2018.1
*Absolute configuration of benzylic carbon is not determined.

### Example 2: Assays for Evaluation of Candidate Compounds

Exemplary compounds of interest are tested, e.g., using the methods described herein, to assess their biological activity including reduction of the deleterious activity of a mutant extended nucleotide repeat (NR) containing target gene in a cell.

### A. Split Gaussia Luciferase Complementation Assay

a. Plasmid Construction

i: pNBR-X1-Supt4-Gluc1 and pNEBR-X1-NGN-Gluc2

The HA-Supt4h and Flag-NGN fragments are amplified by PCR using the plasmid pHA-Supt4h-YC and pFlag-NGN-YN and sub-cloned individually into pcDNA3.1-Gluc1 and pcDNA3.1-Gluc2 (described in “A highly sensitive protein-protein interaction assay based on Gaussia luciferase” published at Nat Methods. 2006 December; 3(12):977-9. Epub 2006 Nov. 12). Then HA-Supt4h-Gluc1 and Flag-NGN-Gluc2 are amplified by PCR and inserted to pNEBR-X1-Hygro (New England BioLabs), which contain RheoSwitch responsive element under the control of RheoSwitch ligand.

ii: pNEBR-X1-Supt4h-G1-NGN-G2

PCR products containing the sequence from 5XRE to polyA in pNEBR-X1-NGN-G2 are inserted to pNEBR-X1-Supt4h-G1 at Pcil site to generate Supt4h-G1 and NGN-G2 bidirectional under their own RheoSwitch responsive element and polyA in the same plasmid.

b. Stable Cloned Cell Line

i: 293-R1 is a cloned cell line which is engineered to constitutively express RSL1 receptor/activator by transfecting HEK 293 cells with pNEBR-R1 plasmid (New England BioLabs) and selected with blasticidin.

ii: M2-8 is a cloned 293-R1 cell which can inducibly express pNEBR-X1-Supt4h-G1-NGN-G2 by addition of RSL1. Two point mutations (M431 and M110I) are introduced to the GL1 and GL2 for better stability according to “A high-throughput cell-based Gaussia luciferase reporter assay for identifying modulators of fibulin-3 secretion” published in J Biomol Screen. 2013 July; 18(6):647-58. The cell line is selected by hygromycin.

c. Cell Culture and Transfection Condition

All the HEK-293 cells and derivative clones are maintained in DMEM containing 10% FBS plus corresponding antibiotics (250 μg/ml hygromycin B, 10 μg/ml blasticidin or both) at 37° C., 5% CO₂. All the transfections are done by using Lipofectamine 2000 (Invitrogen) according to the manufacturer's instructions.

d. Bioluminescence Assay from Cell Lysates

Plasmids harboring the Gluc1 and Gluc2 are co-transfected in a 1:1 ratio into 293-R1 cells plated on tissue culture treated 24-well plates using Lipofectamine 2000 according to the manufacturer's instructions. For stable M2-8 cells, the cells are directly plated into 96-well or 384-well white plates. Twenty-four hours later, RheoSwitch ligand with or without test compound is added to the cells for induction/drug treatment. After 24 hr, the cells are washed with PBS and the plate was stored at −20° C. overnight. Lysis buffer [30 mM Tris-HCl (pH 8.0) 5 mM NaCl, 0.1% Triton X-100] with 10 μg/ml native coelenterazine (Nanolight Technology) is then added to the cells at room temperature for one hour in the dark. After shaking for about 1 min, 40 μl of cell lysate are transferred to a white 96-well plate. For M2-8 in white micro plate, no transfer is needed. Signal intensities (integrated 100 ms) were read on Tecan Infinite M200 or M1000.

The split Gaussia luciferase complementation assay measures the interaction between Sup4h and NGN. NGN is the subunit of Supt5h that binds to Supt4h. The existence of a functional complex of Supt4h and Supt5h has previously been shown to be needed for RNA polymerase I to proceed efficiently though gene regions containing expansions of nucleotide repeats. Interruption of the Supt4h/NGN interaction by a compound is demonstrated by the split Gaussia assay to show if a compound of interest interrupts the formation of the Supt4h/Supt5h complex.

### B. Mutant and Wild-Type HTT Assay in Lymphoblastoid Cells (LBC)

a. Cell Culturing and Compound Treatment

Lymphoblastoid cells derived from a Huntington's disease patient with 250 CAG repeats in the mutant HTT allele (GM14044 from Coriell Institute) were cultured in RPMI 1640 (Corning Cellgro, 10-040-CV) containing fetal bovine serum (Atlanta Biologicals, S11150). 4×10⁵ cells were seeded on a 24-well plate and incubated with testing compound for 3 days. The cell suspension was then transferred to a 1.5 ml Eppendorf tube. Cells were collected by centrifugation and washed once with PBS. After all liquid was removed, the cell pellet was placed at −80° C. for at least 10 minutes. Lysis buffer [30 mM Tris-HCl (pH 8.0), 5 mM NaCl, 0.1% Triton X-100] with the proteinase inhibitor cocktail (P8340 from Sigma-Aldrich) was then added to the cells. Following centrifugation (14 k rpm for 15 min at 4° C.), the supernatant was collected. The protein concentrations were determined by BCA assay (Pierce, ThermoFisher).

b. Western Blot for LBC

Equal amounts of protein were loaded onto 4-12% gel (Invitrogen, WG1403A). After electrophoresis, the gels were transferred to nitrocellulose membranes by wet transfer at 72V for 3 hrs. Total protein stain (LiCor, 926-11011) was used to verify comparable amount of loading protein in membranes. The protein levels of mutant HTT, wild-type HTT, TBP, and tubulin were determined by immunoblotting with anti-polyQ specific antibody (clone MW1), anti-Huntingtin antibody (Abcam, ab45169), anti-TBP antibody (Sigma, T1827) and anti-alpha tubulin antibody (Sigma, T9026). Blots were imaged on a Li-Cor Odyssey infrared imager. The band intensities were determined by Li-Cor Odyssey software.

c. LBC Cell Viability

Cell viability was determined by Cell Titer-Glo reagent (Promega, PRG9243). After 3-day compound treatment, 15 μl of cell suspension was taken out from plate and incubated with 10 μl of CTG reagent. The values of reaction mixture were detected by Tecan.

C. Mutant HTT Assay in Induced Pluripotent Stem Cells (iPSC)

a. Cell Culturing and Compound Treatment

Huntington's disease patient iPSCs (ND50036 from NINDS) were detached into single cells by Accutase (25058CI from Corning), counted and plated on a 24-well plate coated with Matrigel (354277 from Corning) at density of 7,000 cells/well. After 48 hrs, compounds were added to the cell culture medium mTeSR1 (85850 from StemCell Technologies) and the cells were incubated for one day. The medium was then removed, and the cells were washed with PBS. After all liquid was removed, the plate was placed at −80° C. overnight. Lysis buffer [30 mM Tris-HCl (pH 8.0), 5 mM NaCl, 0.1% Triton X-100] with the complete proteinase inhibitor cocktail (5892791001 from Sigma-Aldrich) was then added to the cells. Cell samples were lysed on an orbital plate shaker at 4° C. at 700 rpm for 30 min. The supernatants from spinning (14 k rpm for 10 min) were collected. The protein concentrations were determined by BCA assay (23225 from Pierce, ThermoFisher).

b. Detection of Mutant HTT Protein

Equal amounts of protein lysate collected from each sample were added to a 96-well plate that had been coated with a human HTT specific monoclonal antibody. After washing away any unbound substances, a biotinylated anti-human mutant HTT specific detection antibody was added. Streptavidin-horseradish peroxidase was subsequently added, followed by TMB Substrate Solution, which yielded a blue product that turns yellow when the Stop Solution was added. The intensity of the color measured was in proportion to the amount of mutant HTT bound in the initial step. The mutant HTT values are then interpolated from a standard curve.

### D. Alleviation of Neuron Degeneration Phenotypes of Mutant HTT in Drosophila HD Models

a. Fly Stocks

The Drosophila melanogaster (fruit fly) HD models carry the coding sequence of human HTT exon1 with 97 CAG repeats to mimic mutant HTT of Huntington's disease (HD). The Gmr::HTT97Q fly, expressing mutant HTT primarily in the neurons of Drosophila compound eyes, has a severe degeneration of photoreceptor neurons and the phenotypic trait ‘rough eye’. All of the fly stocks and genetics crosses are maintained at 25° C. on standard cornmeal yeast agar media.

b. Eye Morphology (Rough Eye) Analysis

Fifteen adult male flies (Gmr-HTT97Q/Gmr-HTT97Q or Gmr/Gmr) are crossed with 15 virgin female flies W1118(+/+) in a vial containing standard yeast agar media with the compound in a concentration of 10 μM. Parent-flies are removed from vials at day 7, followed by a collection of newly hatched flies for ‘rough eye’ analysis. The morphology of compound eyes is captured using a Leica DMR upright microscope equipped with a digital camera (CoolSNAP 5.0, Photometrics). To increase the depth of field, imaging software is used to create montage composite images (Helicon Focus, HeliconSoft). A total of 10 flies are collected for analysis in each individual condition, and the biological experiment is conducted three times. DMSO, the reagent solvent of the compound, is included as a control.

### Example 3: Activity of Exemplary Compounds

Exemplary compounds were evaluated, e.g., according to the assays described herein. Activity data for selected compounds is summarized in Table 3.

### Split Gaussia Luciferase Complementation Assay (Table 3).

M2-8 Cell viability Assay (Table 3).
Mutant and wild-type HTT assay in lymphoblastoid cells (LBC) (Table 4).
Mutant HTT assay in induced pluripotent stem cells (iPSC) (Table 4).
Phenotypic analysis of mutant HTT in Drosophila HD model (Table 4).

TABLE 3
Biological activity and Cell Viability of Selected Compounds
A: IC50 ≤100 nM; B: IC50 101-300 nM;
C: IC50 301-1000 nM; and D: IC50 >1000 nM

| | M2-8 Luciferase | |
|---|---|---|
| Compound # | IC50 (nM) | M2-8 Cell viability |
| 1 | A | + |
| 2 | A | + |
| 3 | D | + |
| 4 | D | + |
| 5 | A | + |
| 6 | C | + |
| 7 | C | + |
| 8 | D | + |
| 9 | C | + |
| 10 | D | + |
| 11 | D | + |
| 12 | D | + |
| 13 | A | + |
| 14 | A | + |
| 15 | D | + |
| 16 | D | + |
| 17 | D | + |
| 18 | D | + |
| 19 | A | + |
| 20 | B | + |
| 21 | A | + |
| 22 | B | + |
| 23 | C | + |
| 24 | A | + |
| 25 | B | + |
| 26 | B | + |
| 27 | A | + |
| 28 | A | + |
| 29 | B | + |
| 30 | B | + |
| 31 | A | + |
| 32 | B | + |
| 33 | B | + |
| 34 | D | + |
| 35 | C | + |
| 36 | A | + |
| 37 | B | + |
| 38 | A | + |
| 39 | A | + |
| 40 | B | + |
| 41 | A | + |
| 42 | A | + |
| 43 | A | + |
| 45 | A | + |
| 46 | C | + |
| 47 | B | + |
| 48 | A | + |
| 49 | A | + |
| 50 | A | + |
| 51 | C | + |
| 52 | A | + |
| 53 | B | + |
| 54 | A | + |
| 55 | A | + |
| 56 | A | + |
| 57 | A | + |
| 58 | B | + |
| 59 | B | + |
| 60 | A | + |
| 61 | B | + |
| 62 | B | + |
| 63 | A | + |
| 64 | B | + |
| 65 | A | + |
| 66 | A | + |
| 67 | A | + |
| 68 | A | + |
| 69 | A | + |
| 70 | A | + |
| 71 | A | + |
| 72 | A | + |
| 73 | B | + |

+: cell viability is greater than 50% at the concentration of 1000 nM

TABLE 4
Biological activity of Selected Compounds
A: IC20 ≤100 nM; B: IC20 101-300 nM;
C: IC20 301-1000 nM; D: >1000 nM

| | | | LBC | | iPSC | |
|---|---|---|---|---|---|---|
| | LBC | LBC | via- | iPSC | via- | HD- |
| | IC20 | IC20 | bility | IC20 | bility | Drosophila |
| Compound | (nM) | (nM) | at | (nM) | at | Phenotypic |
| # | mHTT | wtHTT | IC20 | mHTT | IC20 | assay |
| 1 | B | >1000 | * | | | & |
| 2 | A | >250 | * | A | + | & |
| 3 | D | >1000 | | >100 | | @ |
| 5 | A | >1000 | * | >100 | | |
| 13 | B | >1000 | * | A | + | |
| 14 | A | >1000 | * | A | + | |
| 19 | B | >1000 | * | | | |
| 21 | B | >1000 | * | | | |
| 24 | B | >1000 | * | | | |
| 25 | C | >1000 | * | | | |
| 26 | B | >1000 | * | | | |
| 27 | B | >1000 | * | | | |
| 28 | A | >1000 | * | A | + | |
| 31 | A | >250 | * | A | + | |
| 32 | A | >1000 | * | >100 | | |
| 33 | C | >1000 | * | >100 | | |
| 36 | B | >1000 | * | A | + | |
| 37 | B | >1000 | * | >100 | | |
| 38 | B | >1000 | * | A | + | |
| 39 | D | >1000 | | >100 | | |
| 41 | B | >1000 | * | >100 | | |
| 42 | A | >1000 | * | A | + | |
| 43 | B | >1000 | * | A | + | |
| 45 | A | >1000 | * | A | + | |
| 48 | A | >500 | * | A | + | |
| 49 | B | >500 | * | A | + | |
| 50 | A | >500 | * | A | + | |
| 52 | A | >500 | * | A | + | |
| 53 | D | >500 | | >100 | | |
| 58 | B | >1000 | * | >100 | | |
| 59 | B | >1000 | * | >100 | | |
| 60 | B | >1000 | * | A | + | |
| 63 | A | >500 | * | A | + | |
| 65 | C | >1000 | * | A | + | |
| 66 | A | >1000 | * | A | + | |
| 68 | B | >1000 | * | >100 | | |
| 69 | | | | A | + | |
| 70 | | | | A | + | |
| 71 | | | | A | + | |

*: cell viability is greater than 60% at the concentration of IC20
+: cell viability is greater than 75% at the concentration of IC20
&: rough eye phenotype of HD flies is rescued by testing compound greater than 40%
@: rough eye phenotype of HD flies is rescued by testing compound less than 40%

### Example 4: Pharmacokinetic Evaluation of Exemplary Compounds

Selected compounds were evaluated for various pharmacokinetic properties in vitro, including mouse hepatocyte stability assay results shown in Table 5. Also, pharmacokinetic studies were performed in rodents on exemplary compounds, and a summary of the results for selected compounds is shown in Table 6. Based on these results, several of the subject compounds were shown to have acceptable pharmacokinetic properties.

A. In Vitro Metabolic Stability in Hepatocyte

A compound's metabolic stability was assessed using mouse hepatocytes incubated with pre-defined concentration of test articles over several incubation period. The extent of metabolism was calculated from the disappearance of the test compound measured by liquid chromatography mass spectrometry (LC-MS), compared to that of 0-min control. After incubation, quench solutions including internal standards were added and analyzed by LC/MS. The elimination rate constant and intrinsic clearance (CL_{hepatocyte}) values were calculated by linear regression of the compound elimination over the duration of incubation, normalized to the cell number in the given volume of incubation medium.

B. In Vivo Pharmacokinetics and Brain Penetration

The pharmacokinetic assessment was carried out in rodents (e.g. Sprague-Dawley rats or C57BL6 mice) after a single dose administration by IV (e.g., 0.5 mg/kg) or PO (e.g., 5 mg/kg). Biological samples were collected at pre-determined time points, batch-processed, and the test articles were extracted and quantified with LC-MS, utilizing internal standards and separately prepared calibration curves. Blood (or plasma) concentration to time profiles were acquired for compounds and a selected list is presented in Table 6 with the non-compartmental PK parameters (e.g., peak concentration in circulation (Cₘₐₓ), absolute bioavailability (F_{abs}), elimination half-life, clearance (CL), and volume of distribution at steady state (Vdₛₛ)). Upon terminal collection, the brains were also collected and homogenized in an extraction buffer, followed by quantification with LC-MS. After brain concentration levels (ng/g of brain tissue) were determined, the mean ratios of the brain-to-blood (BB) concentration (or brain-to-plasma concentration) at various collection time points were averaged, then shown in Table 6 as BB Ratio to indicate the extent of brain penetration.

TABLE 5
In vitro metabolic stability of exemplary compounds
A: CL_{hepatocyte} <10 μl/min/10⁶cells; B: CL_{hepatocyte} 10-30 μl/min/10⁶cells;
CL_{hepatocyte} >30 μl/min/10⁶cells

| Cmpd # | Mouse HepStability |
|---|---|
| 1 | C |
| 2 | C |
| 28 | A |
| 31 | A |
| 36 | C |
| 37 | B |
| 38 | C |
| 42 | C |
| 49 | B |

TABLE 6
Pharmacokinetic study in rodents following a single dose
Maximum concentration in blood (or plasma);
A: Cₘₐₓ <100 nM; B: Cₘₐₓ 100-300 nM; C: Cₘₐₓ >300 nM.
Concentration ratios of brain-to-blood (BB) were measured at various time points.

| | Pharmacokinetic Analysis Following Single Dose in Rodents | | | | | | |
|---|---|---|---|---|---|---|---|
| | | | | | Elimination | | |
| Cmpd | | | | F_{abs} | Half-life | CL | Vdₛₛ |
| # | Species | Cₘₐₓ | BB_Ratios | (%) | (hr) | (L/hr/kg) | (L/kg) |
| 1 | Rat | B | ≥1 | <30 | 1.9 | 5.1 | 12 |
| 2 | Mouse | A | — | <30 | 0.8 | 11 | 11 |
| 28 | Mouse | C | ≥1 | >70 | 2.4 | 1 | 3.3 |
| 31 | Mouse | B | ≥1 | 30-70 | 2.6 | 3 | 9.1 |
| 42 | Mouse | B | — | <30 | 1.2 | 4.8 | 7.2 |
| 49 | Mouse | C | ≥1 | <30 | 1.1 | 3.3 | 5.1 |

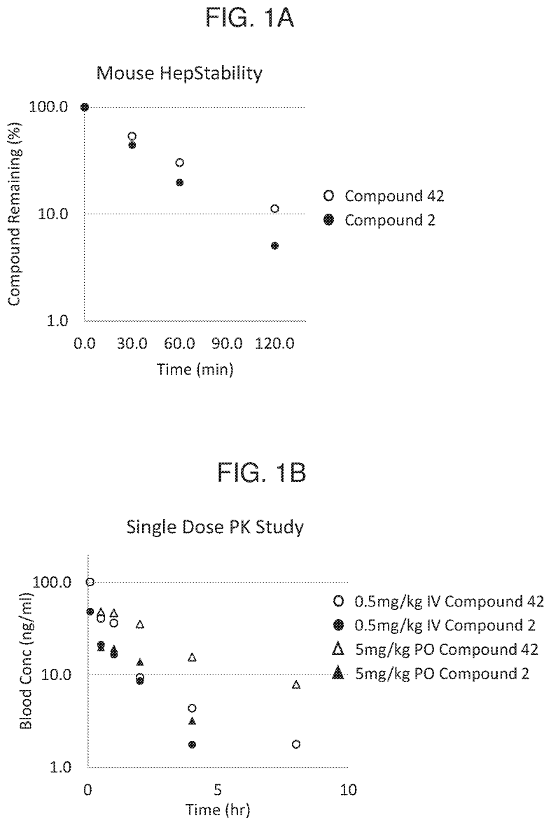

Kinetic isotope effect of deuterium on pharmacokinetic property was observed from in vitro hepatocyte stability assay where non-deuterated compound #2 was more rapidly eliminated than the deuterated version, the compound #42 (FIG. 1A). This was also observed in the in vivo pharmacokinetic study where observed CL showed marked difference, compound #42 exerting a longer elimination half-life (FIG. 1B) following IV administration while showing significantly increased F_{abs} and Cₘₐₓ after oral administration (PO).

FIG. 1A is a graph showing in vitro mouse hepatocyte stability on compound #2 (closed circle) and compound #42 (open circle). FIG. 1B is a graph showing blood concentration-time profile of mouse (N=3 per group) after IV administration of 0.5 mg/kg of compound #2 (closed circle) or #42 (open circle), or PO administration of 5 mg/kg of compound #2 (closed triangle) or #42 (open triangle). In this randomized cross-over study, after a 48-hour washout period the same animals were administered with the opposite compound and resulted in a comparable trend, that is, slower CL of compound #42 than #2.

Notwithstanding the appended claims, the disclosure is also defined by the following clauses:

1. A compound of formula (I):

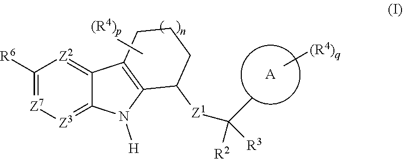

wherein:

A is aryl or heteroaryl;

Z¹ is NR¹, O or S, wherein R¹ is H, alkyl or substituted alkyl;

Z² is CR⁵ or N;

Z⁷ is CR⁷ or N;

Z³ is CR⁸ or N;

R² and R³ are independently selected from H, alkyl and substituted alkyl, or R² and R³ are cyclically linked and together with the carbon atom to which they are attached provide a 3-7 membered carbocycle or heterocycle ring;

each R⁴ and R⁵-R⁸ are independently selected from H, alkyl, substituted alkyl, alkoxy, substituted alkoxy, alkenyl, substituted alkenyl, alkynyl, substituted alkynyl, aryl, substituted aryl, heteroaryl, substituted heteroaryl, heterocycle, substituted heterocycle, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, boronic acid and boronate ester;

n is 0, 1 or 2; and

p and q are independently 0-5;

or a pharmaceutically acceptable salt thereof.

2. The compound of clause 1, wherein A is monocyclic or fused bicyclic aryl or monocyclic heteroaryl.
3. The compound of clause 1 or 2, wherein A is selected from phenyl, 1-naphthyl, 2-naphthyl, 4-imidazolyl, 2-thiophene, 2-furanyl, 2-pyrrolyl, 2-pyridyl, 4-pyridyl and 3-pyridyl.
4. The compound of clause 1, wherein A is of formula (II):

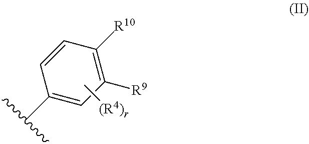

wherein:

r is 0-3; and

R⁹ and R¹⁰ are independently selected from alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring that is optionally further substituted.

5. The compound of any one of clauses 1-4, wherein each R⁴ and R⁵-R¹⁰ are independently selected from H, C₁₋₆alkyl, substituted C₁₋₆alkyl (e.g., C₁₋₆alkoxy-C₁-alkyl, heterocyclyl-C₁₋₆-alkyl, substituted amino-C₁₋₆-alkyl, C₁₋₆alkoxy, substituted C₁₋₆alkoxy, C₁₋₆alkenyl, substituted C₁₋₆alkenyl, C₁₋₆alkynyl, substituted C₁₋₆alkynyl, phenyl, substituted phenyl, heterocycle, substituted heterocycle, halogen, cyano, nitro, hydroxy, —NH₂, sulfoximine, N-substituted sulfoximine, carboxy, sulfonate, C₁₋₆alkanoyl, substituted C₁₋₆alkanoyl, C₁₋₆alkylsulfonamido, substituted C₁₋₆alkylsulfonamido, C₁₋₆alkylamido, substituted C₁₋₆alkylamido, C₁₋₆alkylamino, substituted C₁₋₆alkylamino, C₁₋₆alkyloxycarbonyl, substituted C₁₋₆alkyloxycarbonyl, boronic acid and boronate ester.
6. The compound of clause 5, wherein R⁹ or R¹⁰ has one of the following structures:

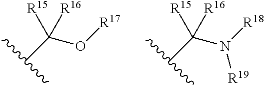

wherein:

R¹⁵ and R¹⁶ are independently selected from H, D, F, (C₁-C₆)alkyl and substituted (C₁-C₆)alkyl, or R¹⁵ and R¹⁶ are cyclically linked and together with the carbon atom to which they are connected provide cycloalkyl or substituted cycloalkyl ring;

R¹⁷ is H, alkyl or substituted alkyl; and

R¹⁸ and R¹⁹ are independently selected from H, alkyl, substituted alkyl, alkylsulfonyl, substituted alkylsulfonyl, alkanoyl, substituted alkanoyl, alkyloxycarbonyl and substituted alkyloxycarbonyl, or R¹⁸ and R¹⁹ are cyclically linked and together with the N atom to which they are attached provide a 5- or 6-membered heterocycle that is optionally further substituted.

7. The compound of clause 6, wherein R⁹ or R¹⁰ has one of the following structures:

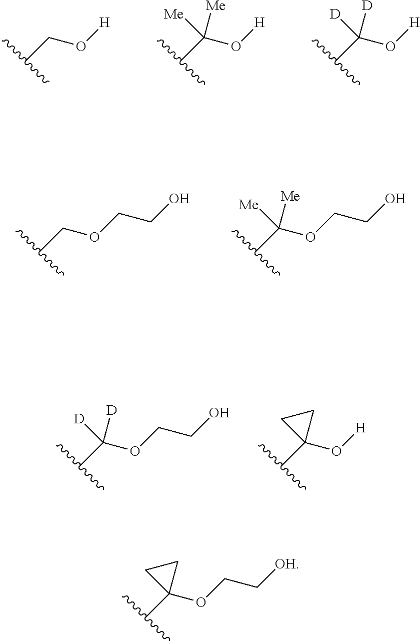

8. The compound of clause 6, wherein R⁹ or R¹⁰ has one of the following structures:

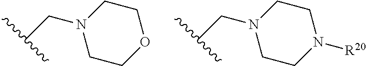

wherein R²⁰ is selected from H, alkyl, substituted alkyl, alkylsulfonyl, substituted alkylsulfonyl, alkanoyl, substituted alkanoyl, alkyloxycarbonyl and substituted alkyloxycarbonyl.
9. The compound of clause 6, wherein R¹⁷ is (C₁-C₆)alkyl or substituted (C₁-C₆)alkyl.
10. The compound of clause 4, wherein A is of formula (IIa):

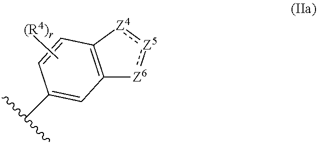

wherein:

Z⁴ is NR¹¹, O or S;

Z⁵ is CR¹² or N;

Z⁶ is CR¹³ or N;

R¹¹ is H, alkyl or substituted alkyl;

R¹² and R¹³ are independently selected from H, alkyl, substituted alkyl, alkoxy, substituted alkoxy, alkynyl, substituted alkynyl, halogen, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl and substituted alkyloxycarbonyl; and

r is 0-3.

11. The compound of clause 10, wherein:

Z⁴ is NR¹;

Z⁵ is N; and

Z⁶ is CR¹³.

12. The compound of clause 4, wherein A is of formula (IIb):

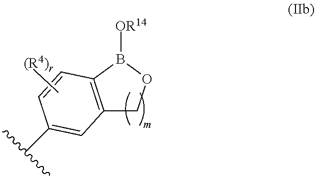

wherein:

R¹⁴ is H, alkyl or substituted alkyl;

m is 1 or 2; and

r is 0-3.

13. The compound of any one of clauses 1-12, wherein R² or R³ is a (C₁-C₆)alkyl or substituted (C₁-C₆)alkyl.
14. The compound of clause 13, wherein:

R² or R³ is —(CH₂)n-R²¹, wherein

R²¹ is halogen (e.g., fluoro) or (C₁-C₆)alkoxy (e.g., methoxy); and

n is 1, 2 or 3.

15. The compound of clause 14, wherein:

R² is H; and

R³ is —CH₂—R²¹ wherein R²¹ is fluoro or methoxy.

16. The compound of any one of clauses 1-12, wherein R² and R³ are cyclically linked and together with the carbon atom to which they are attached provide a cycloalkyl or substituted cycloalkyl (e.g., cyclopropane, cyclobutene or cyclopentane).
17. The compound of clause 16, wherein the cycloalkyl or substituted cycloalkyl is of one of the following structures:

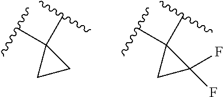 [SMILES: *C1(*)CC1 ; *C1(*)CC1(F)F]

18. The compound of any one of clauses 1-12, wherein R² and R³ are cyclically linked and together with the carbon atom to which they are attached provide a heterocycle (e.g., 4-, 5- or 6-membered heterocycle).
19. The compound of clause 18, wherein the heterocycle is of one of the following structures:

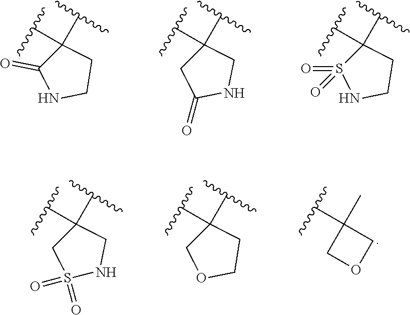

20. The compound of any one of clauses 1-19, wherein Z¹ is NR¹.
21. The compound of any one of clauses 1-19, wherein R¹ is H.
22. The compound of any one of clauses 1-19, wherein Z¹ is O.
23. The compound of any one of clauses 1-22, wherein Z² is CR⁵ and Z³ is CR^{B}.
24. The compound of any one of clauses 1-22, wherein Z² is N and Z³ is CR^{B}.
25. The compound of any one of clauses 1-22, wherein Z² is CR⁵ and Z³ is N.
26. The compound of any one of clauses 1-25, wherein R⁶ is halogen, alkynyl (e.g., —CCH) or substituted alkynyl (e.g., —CC—CH₂OH).
27. The compound of clause 26, wherein R⁶ is Cl, I or Br.
28. The compound of any one of clauses 1-27, wherein R⁷, R⁵ and R⁸ are each H.
29. The compound of any one of clauses 1-28, wherein n is 0.
30. The compound of any one of clauses 1-28, wherein n is 1.
31. The compound of any one of clauses 1-28, wherein n is 2.
32. The compound of any one of clauses 1-31, wherein the compound is enantiomerically enriched in a (1R) stereoisomer.
33. The compound of any one of clauses 1-31, wherein the compound is a (1R) stereoisomer.
34. The compound of clause 1, wherein the compound is of formula (III):

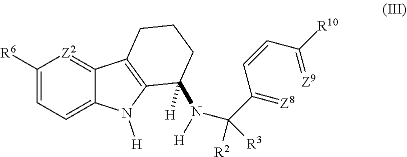

wherein:

Z² is N or OH;

Z⁸ is N or CH;

Z⁹ is N or CR⁹;

R² and R³ are independently selected from H, alkyl and substituted alkyl, or R² and R³ are cyclically linked and together with the carbon atom to which they are attached provide a 3-7 membered carbocycle or heterocycle ring;

R⁶ is selected from halogen, alkynyl and substituted alkynyl;

R⁹ and R¹⁰ are independently selected from H, alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring,

wherein at least one of R⁹ and R¹⁰ is not hydrogen;

wherein at least one of Z⁸ and Z⁹ is not N.

35. The compound of clause 34, wherein the compound is one of formulae (IIIa)-(IIIc):

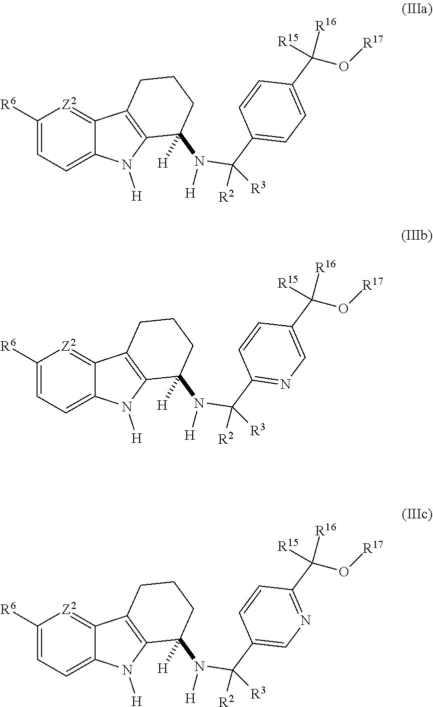

wherein:

R¹⁷ is H, alkyl or substituted alkyl; and

R¹⁵ and R¹⁶ are independently selected from H, D, F, (C₁-C₆)alkyl and substituted (C₁-C₆)alkyl.

36. The compound of clause 35, wherein R¹⁵ and R¹⁶ are each H.
37. The compound of clause 35, wherein R¹⁵ and R¹⁶ are each D.
38. The compound of clause 35, wherein R¹⁵ and R¹⁶ are each (C₁-C₆)alkyl (e.g., methyl).
39. The compound of any one of clauses 35-38, wherein R¹⁷ is H.
40. The compound of any one of clauses 35-38, wherein R¹⁷ is (C₁-C₆)alkyl.
41. The compound of any one of clauses 35-38, wherein R¹⁷ is substituted (C₁-C₆)alkyl (e.g., —CH₂CH₂OH).
42. The compound of any one of clauses 35-41, wherein R² is (C₁-C₆)alkyl or substituted (C₁-C₆)alkyl and R³ is H.
43. The compound of any one of clauses 34-41, wherein R² and R³ are each H.
44. The compound of any one of clauses 34-43, wherein R⁶ is Br.
45. The compound of any one of clauses 34-44, wherein Z² is N.
46. The compound of any one of clauses 34-44, wherein Z² is CH.
47. The compound of clause 45 or 46, wherein the compound is of one of the following structures:

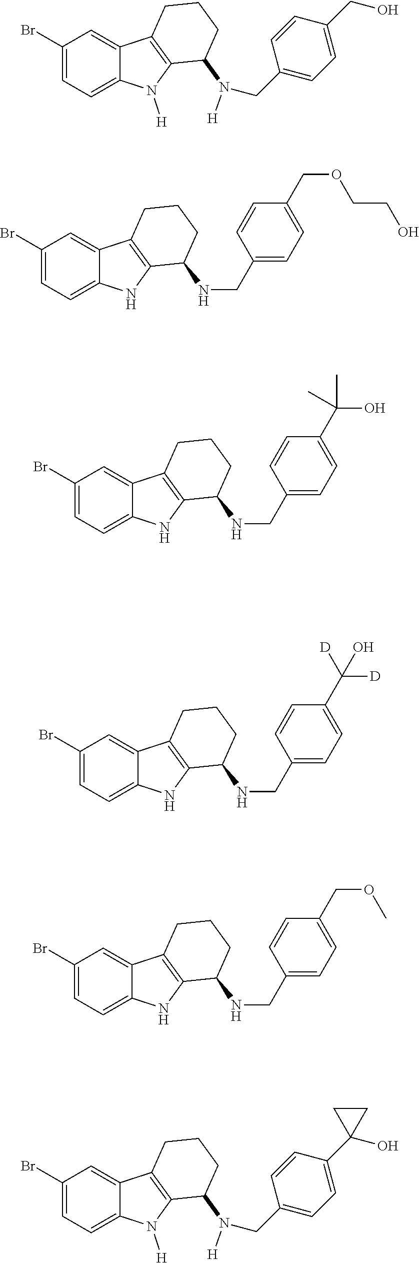 [SMILES: OCc1ccc(CN[C@@H]2CCCc3c2[nH]c2ccc(Br)cc32)cc1 ; OCCOCc1ccc(CN[C@@H]2CCCc3c2[nH]c2ccc(Br)cc32)cc1 ; CC(C)(O)c1ccc(CN[C@@H]2CCCc3c2[nH]c2ccc(Br)cc32)cc1 ; [2H]C([2H])(O)c1ccc(CN[C@@H]2CCCc3c2[nH]c2ccc(Br)cc32)cc1 ; COCc1ccc(CN[C@@H]2CCCc3c2[nH]c2ccc(Br)cc32)cc1 ; OC1(c2ccc(CN[C@@H]3CCCc4c3[nH]c3ccc(Br)cc43)cc2)CC1]

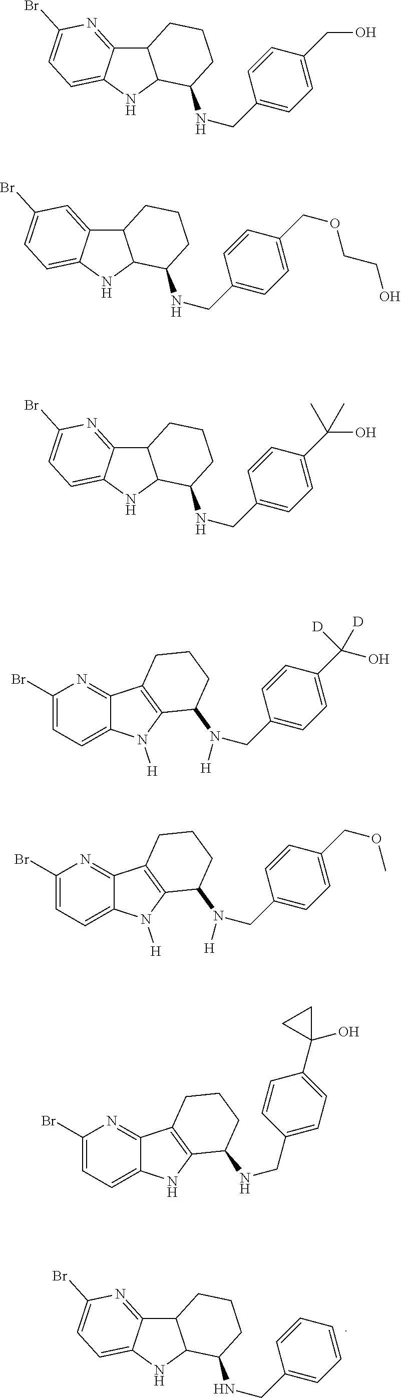

48. The compound of clause 34, wherein the compound is of one of formulae (IVa)-(IVc):

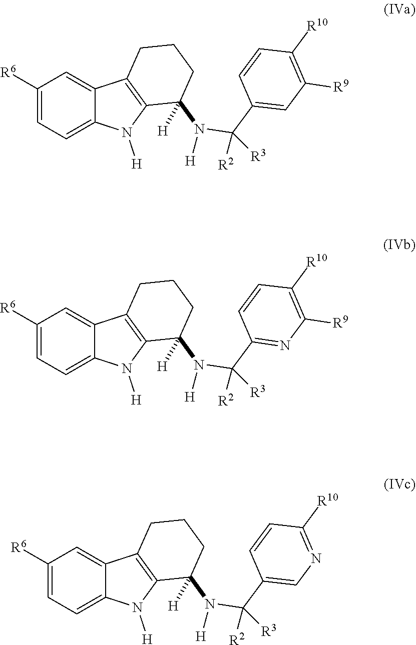

wherein:

R⁹ and R¹⁰ are independently selected from H, —NR^{a}R^{b}, alkoxy, substituted alkoxy, cyano, nitro, halogen, hydroxy, —CONR^{a}R^{b}, —SO₂NR^{a}R^{b}, —CO₂H, —SO₃H, alkylsulfonyl, substituted alkylsulfonyl, alkanoyl, substituted alkanoyl, alkylsulfonamido, substituted alkylsulfonamido, alkylamido, substituted alkylamido, alkyloxycarbonyl, substituted alkyloxycarbonyl, heterocycle, substituted heterocycle, boronic acid and boronate ester; and

R^{a} and R^{b} are independently selected from H, alkyl and substituted alkyl, or R^{a} and R^{b} are cyclically linked and together with the N atom to which they are attached provide a 5- or 6-membered heterocycle that is optionally further substituted.

49. The compound of clause 48, wherein:

either R⁹ is H and R¹⁰ is —NR^{a}R^{b};

or R⁹ is —NR^{a}R^{b} and R¹⁰ is H.

50. The compound of clause 48, wherein R⁹ is H and R¹⁰ is alkoxy or substituted alkoxy.
51. The compound of clause 48, wherein R⁹ is alkoxy or substituted alkoxy and R¹⁰ is H.
52. The compound of clause 48, wherein R⁹ and R¹⁰ are selected from H, cyano, nitro, halogen, —CO₂H and —SO₃H.
53. The compound of clause 48, wherein R⁹ and R¹⁰ are selected from H and —B(OR)₂ wherein each R is independently H, alkyl or substituted alkyl.
54. The compound of any one of clauses 48-53, wherein R² is (C₁-C₆)alkyl or substituted (C₁-C₆)alkyl and R³ is H.
55. The compound of any one of clauses 48-53, wherein R² and R³ are each H.
56. The compound of any one of clauses 48-53, wherein R⁶ is Br.
57. The compound of clause 56, wherein the compound is of one of the following structures:

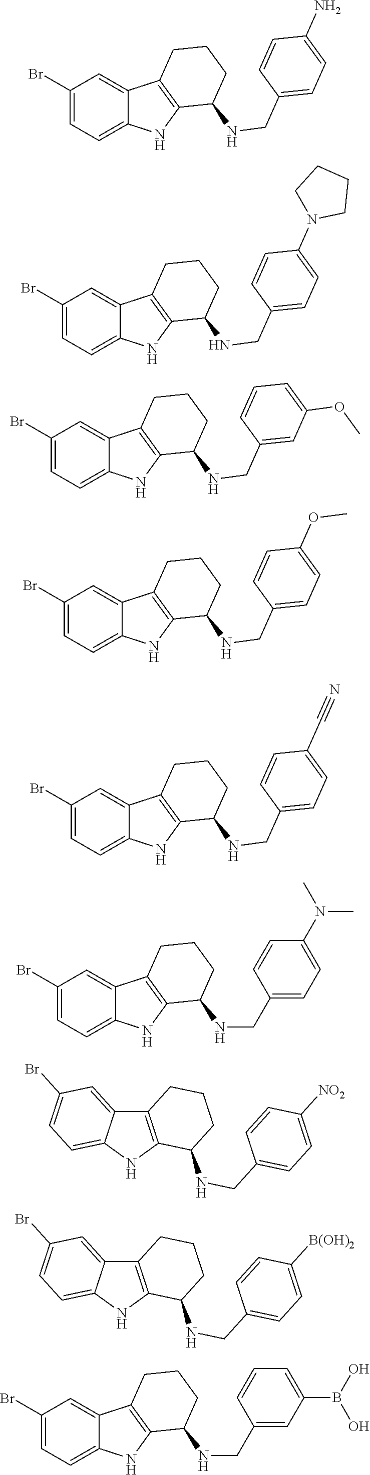

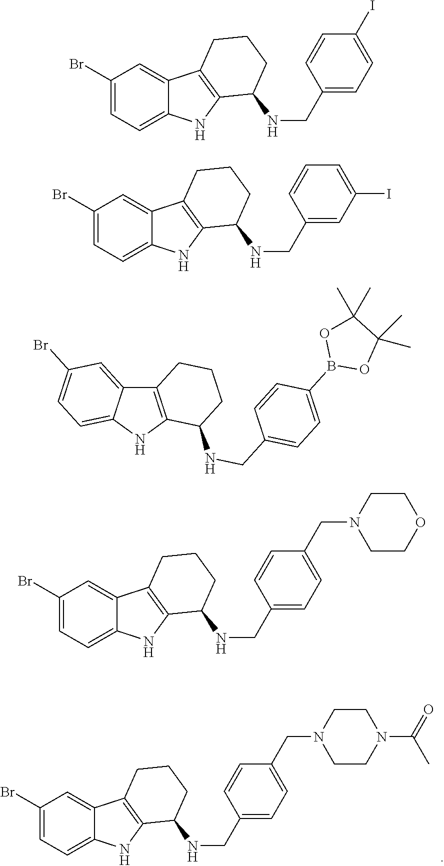

58. The compound of clause 1, wherein the compound is of one of formulae (Va)-(VIII):

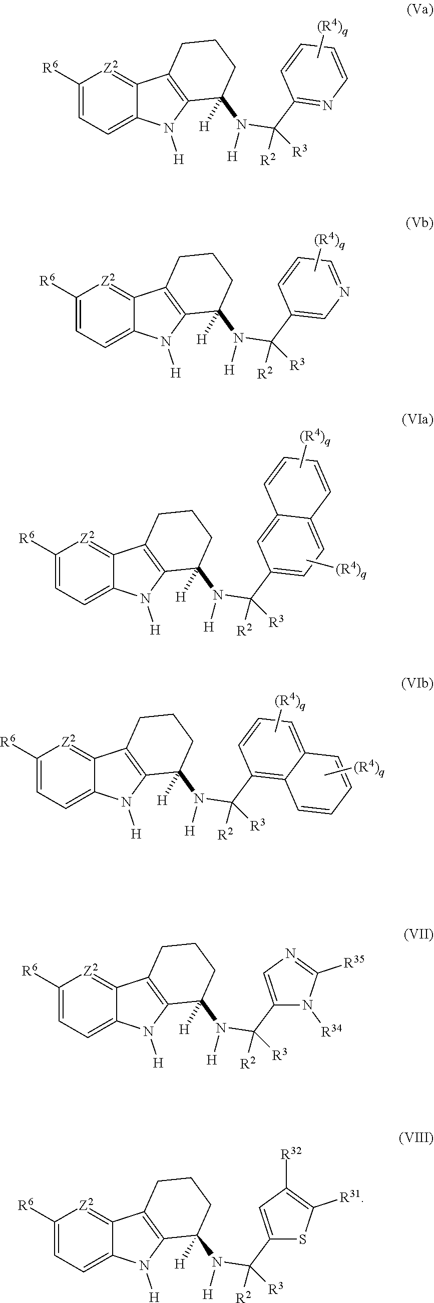

wherein:

each R⁴, R³¹, R³² and R³⁵ are independently selected from H, alkyl, substituted alkyl, alkoxy, substituted alkoxy, alkenyl, substituted alkenyl, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle and substituted heterocycle, or R³¹ and R³² are cyclically linked and together with the carbon atoms to which they are attached provide a fused carbocyclic or heterocyclic ring that is optionally further substituted

R³⁴ is selected from H, alkyl, substituted alkyl, alkylsulfonyl, substituted alkylsulfonyl, alkanoyl, substituted alkanoyl, alkyloxycarbonyl and substituted alkyloxycarbonyl.

59. The compound of clause 58, wherein R² is (C₁-C₆)alkyl or substituted (C₁-C₆)alkyl and R³ is H.
60. The compound of clause 58, wherein R² and R³ are each H.
61. The compound of any one of clauses 58-60, wherein R⁶ is Br.
62. The compound of any one of clauses 58-60, wherein Z² is N.
63. The compound of any one of clauses 58-60, wherein Z² is CH.
64. The compound of clause 62 or 63, wherein the compound is of one of the following structures:

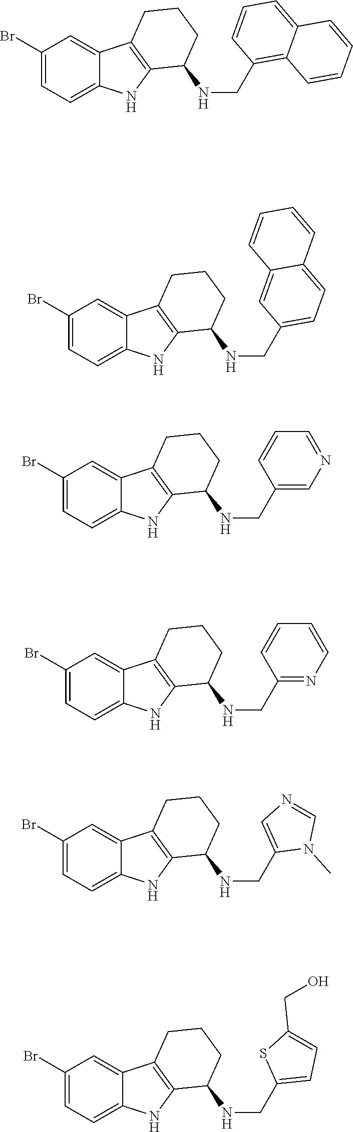 [SMILES: Brc1ccc2[nH]c3c(c2c1)CCC[C@H]3NCc1cccc2ccccc12 ; Brc1ccc2[nH]c3c(c2c1)CCC[C@H]3NCc1ccc2ccccc2c1 ; Brc1ccc2[nH]c3c(c2c1)CCC[C@H]3NCc1cccnc1 ; Brc1ccc2[nH]c3c(c2c1)CCC[C@H]3NCc1ccccn1 ; Cn1cncc1CN[C@@H]1CCCc2c1[nH]c1ccc(Br)cc21 ; OCc1ccc(CN[C@@H]2CCCc3c2[nH]c2ccc(Br)cc32)s1]

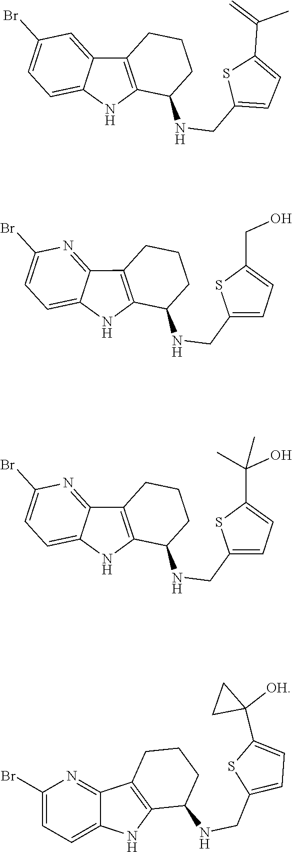 [SMILES: C=C(C)c1ccc(CN[C@@H]2CCCc3c2[nH]c2ccc(Br)cc32)s1 ; OCc1ccc(CN[C@@H]2CCCc3c2[nH]c2ccc(Br)nc32)s1 ; CC(C)(O)c1ccc(CN[C@@H]2CCCc3c2[nH]c2ccc(Br)nc32)s1 ; OC1(c2ccc(CN[C@@H]3CCCc4c3[nH]c3ccc(Br)nc43)s2)CC1]

65. A method of treating a subject for a disease or condition associated with the deleterious impact of a mutant extended nucleotide repeat containing target gene, the method comprising:

administering to a subject in need thereof an effective amount of a compound of formula (I):

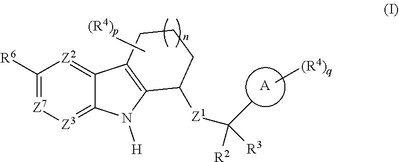

wherein:

A is aryl or heteroaryl;

Z¹ is NR¹, O or S, wherein R¹ is H, alkyl or substituted alkyl;

Z² is CR⁵ or N;

Z⁷ is CR⁷ or N;

Z³ is CR⁸ or N;

R² and R³ are independently selected from H, alkyl and substituted alkyl, or R² and R³ are cyclically linked and together with the carbon atom to which they are attached provide a 3-7 membered carbocycle or heterocycle ring;

each R⁴ and R⁵-R⁸ are independently selected from H, alkyl, substituted alkyl, alkoxy, substituted alkoxy, alkenyl, substituted alkenyl, alkynyl, substituted alkynyl, aryl, substituted aryl, heteroaryl, substituted heteroaryl, heterocycle, substituted heterocycle, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl boronic acid and boronate ester;

n is 0, 1 or 2; and

p and q are independently 0-5;

to treat the subject for a disease or condition associated with the deleterious impact of a mutant extended nucleotide repeat containing target gene.

66. The method according to clause 65, wherein the disease or condition is a neurodegenerative disease.
67. The method according to clause 66, wherein the disease or condition is Huntington's disease.
68. The method according to clause 65, wherein the disease or condition is a neuromuscular dysfunction disease.
69. The method according to clause 65, wherein the disease or condition is selected from spinocerebellar ataxia, dentatorubral pallidoluysian atrophy, amyotrophic lateral sclerosis (ALS), spinal and bular muscular atrophy, myotonic dystrophic type 1 and myotonic dystrophic type 2.
70. The method according to any one of clauses 65-69, wherein the compound is according to any one of clauses 2-64.
71. A method of reducing the deleterious impact of a target gene in a cell, the method comprising:

contacting a cell with an effective amount of a compound of formula (I):

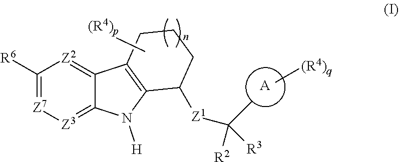

wherein:

A is aryl or heteroaryl;

Z¹ is NR¹, O or S, wherein R¹ is H, alkyl or substituted alkyl;

Z² is CR⁵ or N;

Z⁷ is CR⁷ or N;

Z³ is CR⁸ or N;

R² and R³ are independently selected from H, alkyl and substituted alkyl, or R² and R³ are cyclically linked and together with the carbon atom to which they are attached provide a 3-7 membered carbocycle or heterocycle ring;

each R⁴ and R⁵-R⁸ are independently selected from H, alkyl, substituted alkyl, alkoxy, substituted alkoxy, alkenyl, substituted alkenyl, alkynyl, substituted alkynyl, aryl, substituted aryl, heteroaryl, substituted heteroaryl, heterocycle, substituted heterocycle, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, boronic acid and boronate ester;

n is 0, 1 or 2; and

p and q are independently 0-5;

to reduce the deleterious impact in the cell of a target gene comprising a mutant extended nucleotide repeat (NR) domain.

72. The method according to clause 71, wherein compound reduces expression of a toxic expression product of the target gene.
73. The method according to any one of clauses 71-72, wherein the mutant extended NR domain is a mutant trinucleotide repeat (TNR) domain.
74. The method according to any one of clauses 71-73, wherein the target gene is selected from the group consisting of: ataxin 1, ataxin 2, ataxin 3, ataxin 7, TBP, atrophin 1, androgen receptor protein, huntingtin protein (HTT), C90RF72 and DMPK (e.g., DMPK-1).
75. The method according to any one of clauses 71-74, wherein the compound selectively diminishes interaction of a SPT4 protein and a SPT5 protein in the cell.
76. The method according to any one of clauses 71-75, wherein the compound is according to one of clauses 2-64.
77. A kit, comprising:

a dose of a compound having a structure of formula (I) according to any one of clauses 1-64 in an amount effective to treat a subject for a disease or condition associated with the deleterious impact of a mutant extended nucleotide repeat containing target gene; and

a dose of a second active agent in an amount effective to treat a subject for a disease or condition associated with the deleterious impact of a mutant extended nucleotide repeat containing target gene.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A compound of formula (I): 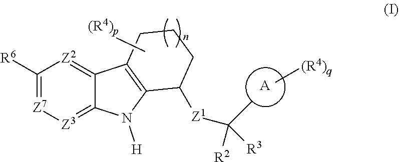
wherein:
A is aryl or heteroaryl;
Z¹ is NR¹, O or S, wherein R¹ is H, alkyl or substituted alkyl;
Z² is CR⁵ or N;
Z⁷ is CR⁷ or N;
Z³ is CR⁸ or N;
R² and R³ are independently selected from H, alkyl and substituted alkyl, or R² and R³ are cyclically linked and together with the carbon atom to which they are attached provide a 3-7 membered carbocycle or heterocycle ring;
each R⁴ and R⁵-R⁸ are independently selected from H, alkyl, substituted alkyl, alkoxy, substituted alkoxy, alkenyl, substituted alkenyl, alkynyl, substituted alkynyl, aryl, substituted aryl, heteroaryl, substituted heteroaryl, heterocycle, substituted heterocycle, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, boronic acid and boronate ester;
n is 0, 1 or 2; and
p and q are independently 0-5;
or a pharmaceutically acceptable salt thereof.

2. The compound of claim 1, wherein A is monocyclic or fused bicyclic aryl or monocyclic heteroaryl.

3. The compound of claim 1, wherein A is selected from phenyl, 1-naphthyl, 2-naphthyl, 4-imidazolyl, 2-thiophene, 2-furanyl, 2-pyrrolyl, 2-pyridyl, 4-pyridyl and 3-pyridyl.

4. The compound of claim 1, wherein A is of formula (II): 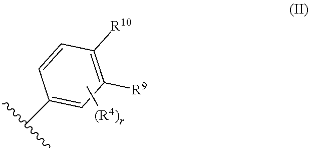
wherein:
r is 0-3; and
R⁹ and R¹⁰ are independently selected from alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring that is optionally further substituted.

5. The compound of claim 1, wherein each R⁴ and R⁵-R¹⁰ are independently selected from H, C₁₋₆alkyl, substituted C₁₋₆alkyl (e.g., C₁₋₆alkoxy-C₁₋₆alkyl, heterocyclyl-C₁₋₆alkyl, substituted amino-C₁₋₆alkyl, C₁₋₆alkoxy, substituted C₁₋₆alkoxy, C₁₋₆alkenyl, substituted C₁₋₆alkenyl, C₁₋₆alkynyl, substituted C₁₋₆alkynyl, phenyl, substituted phenyl, heterocycle, substituted heterocycle, halogen, cyano, nitro, hydroxy, —NH₂, sulfoximine, N-substituted sulfoximine, carboxy, sulfonate, C₁₋₆alkanoyl, substituted C₁₋₆alkanoyl, C₁₋₆alkylsulfonamido, substituted C₁₋₆alkylsulfonamido, C₁₋₆alkylamido, substituted C₁₋₆alkylamido, C₁₋₆alkylamino, substituted C₁₋₆alkylamino, C₁₋₆alkyloxycarbonyl, substituted C₁₋₆alkyloxycarbonyl, boronic acid and boronate ester.

6. The compound of claim 1, wherein the compound is of formula (III): 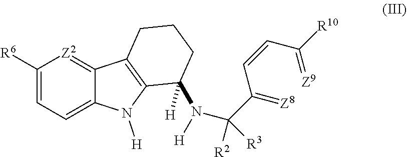
wherein:
Z² is N or CH;
Z⁸ is N or CH;
Z⁹ is N or CR⁹;
R² and R³ are independently selected from H, alkyl and substituted alkyl, or R² and R³ are cyclically linked and together with the carbon atom to which they are attached provide a 3-7 membered carbocycle or heterocycle ring;
R⁶ is selected from halogen, alkynyl and substituted alkynyl;
R⁹ and R¹⁰ are independently selected from H, alkyl, substituted alkyl, alkoxy, substituted alkoxy, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle, substituted heterocycle, boronic acid and boronate ester, or R⁹ and R¹⁰ are cyclically linked and together with the carbon atoms to which they are attached provide a fused carbocyclic or heterocyclic ring,
wherein at least one of R⁹ and R¹⁰ is not hydrogen;
wherein at least one of Z⁸ and Z⁹ is not N.

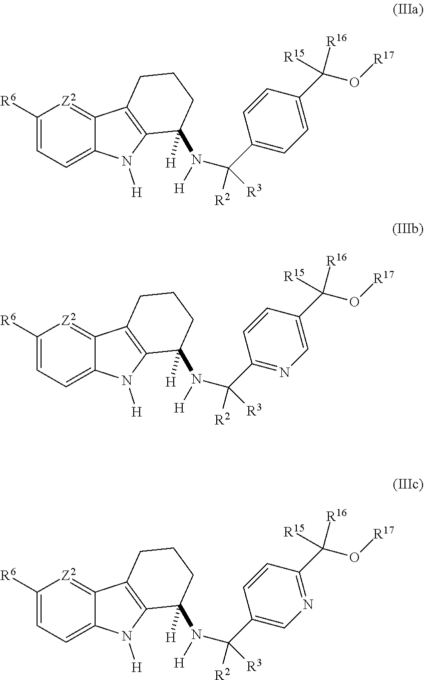

7. The compound of claim 6, wherein the compound is one of formulae (IIIa)-(IIIc):
wherein:
R¹⁷ is H, alkyl or substituted alkyl; and
R¹⁵ and R¹⁶ are independently selected from H, D, F, (C₁-C₆)alkyl and substituted (C₁-C₆)alkyl.

8. The compound of claim 6, wherein the compound is of one of formulae (IVa)-(IVc): 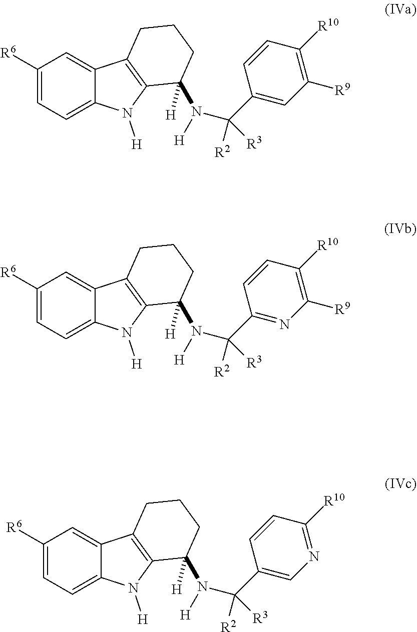
wherein:
R⁹ and R¹⁰ are independently selected from H, —NR^{a}R^{b}, alkoxy, substituted alkoxy, cyano, nitro, halogen, hydroxy, —CONR^{a}R^{b}, —SO₂NR^{a}R^{b}, —CO₂H, —SO₃H, alkylsulfonyl, substituted alkylsulfonyl, alkanoyl, substituted alkanoyl, alkylsulfonamido, substituted alkylsulfonamido, alkylamido, substituted alkylamido, alkyloxycarbonyl, substituted alkyloxycarbonyl, heterocycle, substituted heterocycle, boronic acid and boronate ester; and
R^{a} and R^{b} are independently selected from H, alkyl and substituted alkyl, or R^{a} and R^{b} are cyclically linked and together with the N atom to which they are attached provide a 5- or 6-membered heterocycle that is optionally further substituted.

9. The compound according to claim 8, wherein R⁶ is Br.

10. The compound of claim 9, wherein the compound is of one of the following structures:

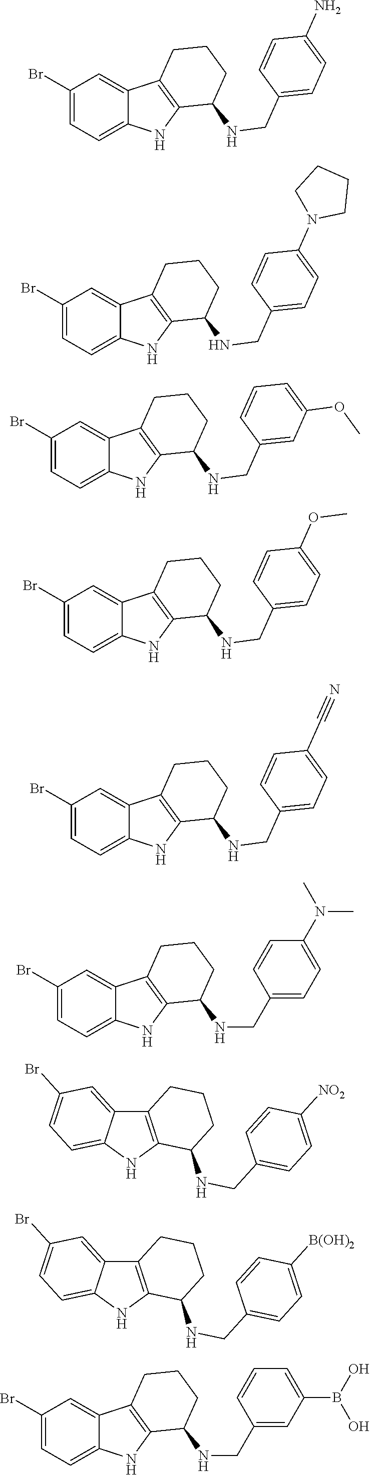

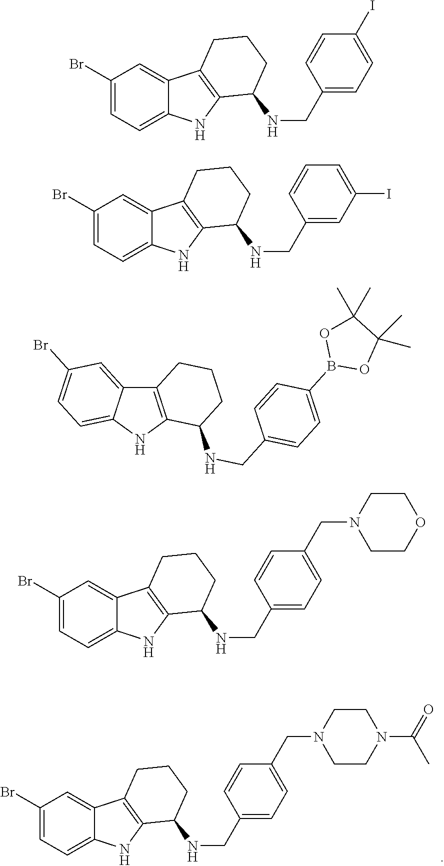

11. The compound of claim 1, wherein the compound is of one of formulae (Va)-(VIII): 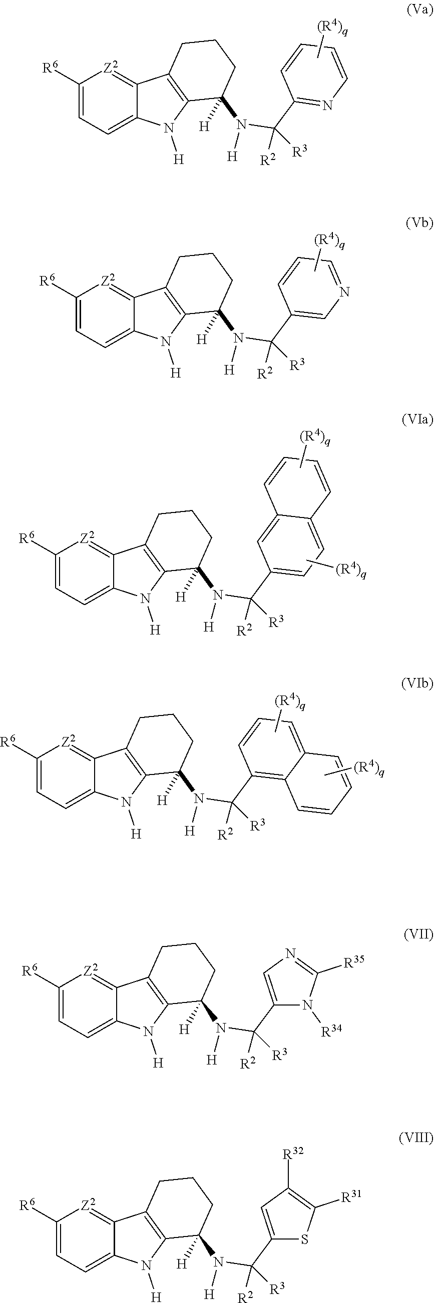
wherein:
each R⁴, R³¹, R³² and R³⁵ are independently selected from H, alkyl, substituted alkyl, alkoxy, substituted alkoxy, alkenyl, substituted alkenyl, alkynyl, substituted alkynyl, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl, heterocycle and substituted heterocycle, or R³¹ and R³² are cyclically linked and together with the carbon atoms to which they are attached provide a fused carbocyclic or heterocyclic ring that is optionally further substituted
R³⁴ is selected from H, alkyl, substituted alkyl, alkylsulfonyl, substituted alkylsulfonyl, alkanoyl, substituted alkanoyl, alkyloxycarbonyl and substituted alkyloxycarbonyl.

12. A method of treating a subject for a disease or condition associated with the deleterious impact of a mutant extended nucleotide repeat containing target gene, the method comprising:
administering to a subject in need thereof an effective amount of a compound of formula (I): 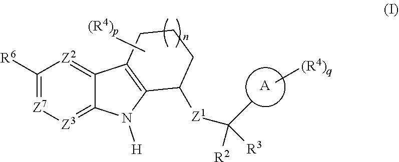
wherein:
A is aryl or heteroaryl;
Z¹ is NR¹, O or S, wherein R¹ is H, alkyl or substituted alkyl;
Z² is CR⁵ or N;
Z⁷ is CR⁷ or N;
Z³ is CR⁸ or N;
R² and R³ are independently selected from H, alkyl and substituted alkyl, or R² and R³ are cyclically linked and together with the carbon atom to which they are attached provide a 3-7 membered carbocycle or heterocycle ring;
each R⁴ and R⁵-R⁸ are independently selected from H, alkyl, substituted alkyl, alkoxy, substituted alkoxy, alkenyl, substituted alkenyl, alkynyl, substituted alkynyl, aryl, substituted aryl, heteroaryl, substituted heteroaryl, heterocycle, substituted heterocycle, halogen, nitro, cyano, hydroxy, —NH₂, substituted amino, amido, sulfonamide, sulfoximine, N-substituted sulfoximine, carboxy, sulfonate, alkylsulfonyl, substituted alkylsulfonyl, alkanoyl, substituted alkanoyl, alkylsulfonamido, substituted alkylsulfonamido, alkylamido, substituted alkylamido, alkylamino, substituted alkylamino, alkyloxycarbonyl, substituted alkyloxycarbonyl boronic acid and boronate ester;
n is 0, 1 or 2; and
p and q are independently 0-5;
to treat the subject for a disease or condition associated with the deleterious impact of a mutant extended nucleotide repeat containing target gene.

13. The method according to claim 12, wherein the disease or condition is a neurodegenerative disease.

14. The method according to claim 13, wherein the disease or condition is Huntington's disease.

15. A kit, comprising:
a dose of a compound having a structure of formula (I) according to claim 1 in an amount effective to treat a subject for a disease or condition associated with the deleterious impact of a mutant extended nucleotide repeat containing target gene; and
a dose of a second active agent in an amount effective to treat a subject for a disease or condition associated with the deleterious impact of a mutant extended nucleotide repeat containing target gene.
